(19) United States
(12) Patent Application Publication
(10) Pub. No.: US 2003/0077804 A1
(43) Pub. Date: Apr. 24, 2003

(54) Compositions and methods for recombinational cloning of nucleic acid molecules

(75) Inventors: Devon R.N. Byrd, Waynesville, NC (US); Dominic Esposito, Columbia, MD (US)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Invitrogen Corporation

(21) Appl. No.: 10/125,648

(22) Filed: Apr. 19, 2002

Related U.S. Application Data
(60) Provisional application No. 60/284,528, filed on Apr. 19, 2001.

(51) Int. Cl. C12Q 1/68; C07H 21/04; C12N 9/22; C12P 21/02; C12N 1/21; C12N 15/74

(52) U.S. Cl. 435/199; 435/6; 435/69.1; 435/252.33; 435/320.1; 536/23.2

(57) ABSTRACT

The present invention relates generally to recombinant DNA technology. The invention relates more specifically to compositions and methods for recombinational cloning of nucleic acid molecules using recombination systems. In particular, the invention relates to compositions comprising one or more Fis proteins and one or more additional components used for recombinational cloning (such as one or more recombination proteins). The invention further relates to the use of the above compositions in methods of recombinational cloning of nucleic acid molecules. The invention also relates to isolated nucleic acid molecules produced by methods of the invention, to vectors comprising such nucleic acid molecules, and to host cells comprising such nucleic acid molecules and vectors.

### CROSS-REFERENCE TO RELATED APPLICATIONS

The present application claims the benefit of the filing date of U.S. Provisional Application No. 60/284,528, filed Apr. 19, 2001, the disclosure of which is incorporated herein by reference in its entirety.

### BACKGROUND OF THE INVENTION

1. Field of the Invention

The present invention relates generally to recombinant DNA technology. The invention relates more specifically to compositions and methods for recombinational cloning of nucleic acid molecules using recombination systems. In particular, the invention relates to compositions comprising one or more Fis proteins and one or more additional components used for recombinational cloning (such as one or more recombination proteins). The invention further relates to the use of the above compositions in methods of recombinational cloning of nucleic acid molecules. The invention also relates to isolated nucleic acid molecules produced by methods of the invention, to vectors comprising such nucleic acid molecules, and to host cells comprising such nucleic acid molecules and vectors.

2. Related Art

Site-Specific Recombinases

Site-specific recombinases are proteins that are present in many organisms (e.g., viruses and bacteria) and have been characterized to have both endonuclease and ligase properties. These recombinases (along with associated proteins in some cases) recognize specific sequences of bases in DNA and exchange the DNA segments flanking those segments. The recombinases and associated proteins are collectively referred to as “recombination proteins” (see, e.g., Landy, A., Current Opinion in Biotechnology 3:699-707 (1993)).

Numerous recombination systems from various organisms have been described. See, e.g., Hoess et al., Nucleic Acids Research 14(6):2287 (1986); Abremski et al., J. Biol. Chem. 261(1):391 (1986); Campbell, J. Bacteriol. 174(23):7495 (1992); Qian et al., J. Biol. Chem. 267(11):7794 (1992); Araki et al., J. Mol. Biol. 225(1):25 (1992); Maeser and Kahnmann Mol. Gen. Genet. 230:170-176) (1991); Esposito et al., Nucl. Acids Res. 25(18):3605 (1997).

Many of these belong to the integrase family of recombinases (Argos et al. EMBO J. 5:433-440 (1986)). Perhaps the best studied of these are the Integrase/att system from bacteriophage λ (Landy, A. Current Opinions in Genetics and Devel. 3:699-707 (1993)), the Cre/loxP system from bacteriophage P1 (Hoess and Abremski (1990) In Nucleic Acids and Molecular Biology, vol.4. Eds.: Eckstein and Lilley, Berlin-Heidelberg: Springer-Verlag; pp. 90-109), and the FLP/FRT system from the Saccharomyces cerevisiae 2μ circle plasmid (Broach et al. Cell 29:227-234 (1982)).

Backman (U.S. Pat. No. 4,673,640) discloses the in vivo use of λ recombinase to recombine a protein producing DNA segment by enzymatic site-specific recombination using wild-type recombination sites attB and attP.

Hasan and Szybalski (Gene 56:145-151 (1987)) discloses the use of λ Int recombinase in vivo for intramolecular recombination between wild type attP and attB sites which flank a promoter. Because the orientations of these sites are inverted relative to each other, this causes an irreversible flipping of the promoter region relative to the gene of interest.

Palazzolo et al. Gene 88:25-36 (1990), discloses phage lambda vectors having bacteriophage λ arms that contain restriction sites positioned outside a cloned DNA sequence and between wild-type loxP sites. Infection of E. coli cells that express the Cre recombinase with these phage vectors results in recombination between the loxP sites and the in vivo excision of the plasmid replicon, including the cloned cDNA.

Pósfai et al. (Nucl. Acids Res. 22:2392-2398 (1994)) discloses a method for inserting into genomic DNA partial expression vectors having a selectable marker, flanked by two wild-type FRT recognition sequences. FLP site-specific recombinase as present in the cells is used to integrate the vectors into the genome at predetermined sites. Under conditions where the replicon is functional, this cloned genomic DNA can be amplified.

Bebee et al. (U.S. Pat. No. 5,434,066) discloses the use of site-specific recombinases such as Cre for DNA containing two loxP sites is used for in vivo recombination between the sites.

Boyd (Nucl. Acids Res. 21:817-821 (1993)) discloses a method to facilitate the cloning of blunt-ended DNA using conditions that encourage intermolecular ligation to a dephosphorylated vector that contains a wild-type loxP site acted upon by a Cre site-specific recombinase present in E. coli host cells.

Waterhouse et al. (PCT No. 93/19172 and Nucleic Acids Res. 21 (9):2265 (1993)) disclose an in vivo method where light and heavy chains of a particular antibody were cloned in different phage vectors between loxP and loxP 511 sites and used to transfect new E. coli cells. Cre, acting in the host cells on the two parental molecules (one plasmid, one phage), produced four products in equilibrium: two different cointegrates (produced by recombination at either loxP or loxP 511 sites), and two daughter molecules, one of which was the desired product.

In contrast to the other related art, Schlake & Bode (Biochemistry 33:12746-12751 (1994)) discloses an in vivo method to exchange expression cassettes at defined chromosomal locations, each flanked by a wild type and a spacer-mutated FRT recombination site. A double-reciprocal crossover was mediated in cultured mammalian cells by using this FLP/FRT system for site-specific recombination.

Transposases

The family of enzymes, the transposases, has also been used to transfer genetic information between replicons. Transposons are structurally variable, being described as simple or compound, but typically encode the recombinase gene flanked by DNA sequences organized in inverted orientations. Integration of transposons can be random or highly specific. Representatives such as Tn7, which are highly site-specific, have been applied to the in vivo movement of DNA segments between replicons (Lucklow et al., J. Virol. 67:4566-4579 (1993)).

Devine and Boeke Nucl. Acids Res. 22:3765-3772 (1994), discloses the construction of artificial transposons for the insertion of DNA segments, in vitro, into recipient DNA molecules. The system makes use of the integrase of yeast TY1 virus-like particles. The DNA segment of interest is cloned, using standard methods, between the ends of the transposon-like element TY1. In the presence of the TY1 integrase, the resulting element integrates randomly into a second target DNA molecule.

DNA Cloning

The cloning of DNA segments currently occurs as a daily routine in many research labs and as a prerequisite step in many genetic analyses. The purpose of these clonings is various, however, two general purposes can be considered: (1) the initial cloning of DNA from large DNA or RNA segments (chromosomes, YACs, PCR fragments, mRNA, etc.), done in a relative handful of known vectors such as pUC, pGem, pBlueScript, and (2) the subcloning of these DNA segments into specialized vectors for functional analysis. A great deal of time and effort is expended in the transfer of DNA segments from the initial cloning vectors to the more specialized vectors. This transfer is called subcloning.

The basic methods for cloning have been known for many years and have changed little during that time. A typical cloning protocol is as follows:

(1) digest the DNA of interest with one or two restriction enzymes;

(2) gel purify the DNA segment of interest when known;

(3) prepare the vector by cutting with appropriate restriction enzymes, treating with alkaline phosphatase, gel purify etc., as appropriate;

(4) ligate the DNA segment to the vector, with appropriate controls to eliminate background of uncut and self-ligated vector;

(5) introduce the resulting vector into an E. coli host cell;

(6) pick selected colonies and grow small cultures overnight;

(7) make DNA minipreps; and

(8) analyze the isolated plasmid on agarose gels (often after diagnostic restriction enzyme digestions) or by PCR.

The specialized vectors used for subcloning DNA segments are functionally diverse. These include but are not limited to: vectors for expressing genes in various organisms; for regulating gene expression; for providing tags to aid in protein purification or to allow tracking of proteins in cells; for modifying the cloned DNA segment (e.g., generating deletions); for the synthesis of probes (e.g., riboprobes); for the preparation of templates for DNA sequencing; for the identification of protein coding regions; for the fusion of various protein-coding regions; to provide large amounts of the DNA of interest, etc. It is common that a particular investigation will involve subcloning the DNA segment of interest into several different specialized vectors.

As known in the art, simple subclonings can be done in one day (e.g., the DNA segment is not large and the restriction sites are compatible with those of the subcloning vector). However, many other subclonings can take several weeks, especially those involving unknown sequences, long fragments, toxic genes, unsuitable placement of restriction sites, high backgrounds, impure enzymes, etc. Subcloning DNA fragments is thus often viewed as a chore to be done as few times as possible. Several methods for facilitating the cloning of DNA segments have been described, e.g., as in the following references.

Ferguson, J., et al. Gene 16:191 (1981), discloses a family of vectors for subcloning fragments of yeast DNA. The vectors encode kanamycin resistance. Clones of longer yeast DNA segments can be partially digested and ligated into the subcloning vectors. If the original cloning vector conveys resistance to ampicillin, no purification is necessary prior to transformation, since the selection will be for kanamycin.

Hashimoto-Gotoh, T., et al. Gene 41:125 (1986), discloses a subcloning vector with unique cloning sites within a streptomycin sensitivity gene; in a streptomycin-resistant host, only plasmids with inserts or deletions in the dominant sensitivity gene will survive streptomycin selection.

Accordingly, traditional subcloning methods, using restriction enzymes and ligase, are time consuming and relatively unreliable. Considerable labor is expended, and if two or more days later the desired subclone can not be found among the candidate plasmids, the entire process must then be repeated with alternative conditions attempted. Although site specific recombinases have been used to recombine DNA in vivo, the successful use of such enzymes in vitro was expected to suffer from several problems. For example, the site specificities and efficiencies were expected to differ in vitro; topologically-linked products were expected; and the topology of the DNA substrates and recombination proteins was expected to differ significantly in vitro (see, e.g., Adams et al, J. Mol. Biol. 226:661-73 (1992)). Reactions that could go on for many hours in vivo were expected to occur in significantly less time in vitro before the enzymes became inactive. Multiple DNA recombination products were expected in the biological host used, resulting in unsatisfactory reliability, specificity or efficiency of subcloning. Thus, in vitro recombination reactions were not expected to be sufficiently efficient to yield the desired levels of product.

Fis Proteins

Fis is a homodimeric protein found in Escherichia coli and Salmonella typhimurium, as well as many other prokaryotes (e.g., Klebsiella pneumoniae, Vibrio cholera, Haemophilus influenza, Pseudomonas aeruginosa, etc.). This protein varies in size generally between about 90 and 110 amino acids. Fis was first identified due to its role in regulating DNA recombination reactions carried out by the DNA invertase family (Johnson, R. C. et al. (1986) Cell 46:531-9 and Koch, C. and Kahmann, R. (1986) J. Biol. Chem. 261:15673-8). Fis is a member of a group of proteins known as the NAPS, or nucleoid-associated proteins, which perform numerous regulatory functions in the cell, and are often isolated as part of the mass of protein-DNA which forms the E. coli nucleoid (Pan, C. Q. et al. (1996) J. Mol. Biol. 264:675-95). Most members of this family appear to be involved in specific or non-specific DNA interactions involving bending, looping, or condensation of the DNA substrate. Other roles for Fis were later identified, including its function as a transcriptional activator of a wide number of promoters (Nilsson, L. et al. (1990) EMBO J. 9:727-34; Ross, W. et al. (1990) EMBO J. 9:3733-42; Xu, J. and Johnson, R. C. (1995) J. Bacteriol. 177:5222-31), a repressor of another set of promoters (Ball, C. A. et al. (1992) J. Bacteriol. 174:8043-56; Koch, C. et al. (1991) Nucl. Acids Res. 19:5915-22; Xu, J. and Johnson, R. C. (1995) J. Bacteriol. 177:938-47), a cofactor for DNA replication (Filutowicz, M. et al. (1992) J. Bacteriol. 174:398-407) and cell division/chromosome separation (Paull, T. T. and Johnson, R. C. (1995) J. Biol. Chem. 270:8744-54), and a participant in site-specific recombination of bacteriophage lambda (Thompson, J. F. et al. (1987) Cell 50:901-8; Ball, C. A. and Johnson, R. C. (1991) J. Bacteriol. 173:4027-31; Ball, C. A. and Johnson, R. C. (1991) J. Bacteriol. 173:4032-8). Cellular levels of Fis vary dramatically during the E. coli cell cycle depending on the growth stage and the availability of nutrients (Ball, C. A. et al. (1992) J. Bacteriol. 174:8043-56; Thompson, J. F. et al. (1987) Cell 50:901-8). Calculations predict that during log phase growth, enough Fis is present in cells to bind every 500 base pairs along the chromosome. However, as cells enter stationary phase or are deprived of nutrients, levels of Fis drop to almost undetectable amounts (Ball, C. A. et al. (1992) J. Bacteriol. 174:8043-56).

Fis is capable of non-specific binding to DNA in vitro, but it has a considerably higher affinity for a series of sites with a degenerate 15 base pair consensus sequence which loosely resembles an inverted repeat (Pan, C. Q. et al. (1996) J. Mol. Biol. 264:675-95; Bruist, M. F. et al. (1987) Genes Dev. 1:762-72; Bokal, A. J. et al. (1995) J. Mol. Biol. 245:197-207).

DNA footprinting shows clear contacts between the protein and the DNA in these 15 base pair Fis binding sites; however, the DNA sequence alone appears to be a poor predictor of Fis binding affinity, and local DNA structure may influence the activity of a given Fis binding site. Fis bends DNA upon specific binding, and the degree of bending appears to depend upon the particular Fis binding site (Thompson, J. F. and Landy, A. (1988) Nucl. Acids Res. 16:9687-9705.; Pan, C. Q. et al. (1996) Biochemistry 35:4326-33). Bend angles between 45 and 90 degrees have been observed in different experiments using different DNA substrates (Thompson, J. F. and Landy, A. (1988) Nucl. Acids Res. 16:9687-9705).

Genetic evidence from Ball and Johnson (Ball, C. A. and Johnson, R. C. (1991) J. Bacteriol. 173:4027-31; Ball, C. A. and Johnson, R. C. (1991) J. Bacteriol. 173:4032-8) demonstrated that not only could Fis stimulate excision of phage lambda, but that lysogeny was also enhanced by the presence of Fis. These experiments, carried out in vivo using phage mutated in the F site and/or E. coli lacking Fis, demonstrated a 15-fold drop in lysogenization frequency when Fis was deleted (Ball, C. A. and Johnson, R. C. (1991) J. Bacteriol. 173:4032-8). A part of this decrease is apparently due to the loss of Fis as a regulator in non-recombination related events. However, a mutation of the F site which eliminates Fis binding without affecting Xis binding, still leads to a loss of 2-3 fold in lysogenization frequency, suggesting that Fis plays a role in integration as well as excision. Experiments carried out in vitro with Fis to look at integration did not identify any effect of Fis on the reaction (Thompson, J. F. et al. (1987) Cell 50:901-908).

Ribosomal Proteins

Characterization

E. coli ribosomes have some 53 different proteins, 21 associated with the 30S subunit (designated S1 through S21) and 32 associated with the 50S subunit (designated L1 through L34). Generally, the lower the number the higher the molecular weight. With the exception of S1 through S4 and L1 through L4, they contain less than 200 amino acids (molecular weights are less than 20 kDa). The primary amino acid sequence of each protein is known. The three-dimensional structures of S5, S6, S8, S17, L1, L7, L9, L14, and L30 are known. Most of these proteins have a relatively high proportion of the two basic amino acids arginine (arg or R) and lysine (lys or K). This intuitively makes sense if most of the ribosomal proteins are assumed to be RNA binding proteins. Much of what is known about ribosomal proteins has been summarized in a series of articles in Annual Reviews of Biochemistry:51:155 (1982); 52:35 (1983); 53:75 (1984); 54:507 (1985); 66:679 (1997).

Enhancement of Yeast Recombination Systems

The yeast FLP/FRT recombination system requires only the FRT DNA binding site and FLP recombinase to carry out recombination. In contrast, the minimum requirements for carrying out recombination in the λ integrase (Int) system include a recombinase (Int) and DNA sites (att), but also IHF protein.

IHF is a member of the HU family of small DNA binding proteins.

These are basic proteins of 100 amino acids or less that bind to DNA and condense its structure. HU will substitute for IHF in the λ recombination system. While IHF and HU do not stimulate the yeast FLP/FRT recombination system, the E. coli ribosomal proteins S3, S4, S5, and L2 do (Bruckner and Cox, Nucl. Acids Res. 17:3145-3161 (1989)). The E. coli ribosomal proteins that have been shown to stimulate the yeast FLP/FRT recombination system are large, all possessing, with one exception, more than 200 amino acids (Table 1); smaller E. coli ribosomal proteins have not been shown to stimulate the FLP/FRT (or any other) recombination system.

TABLE 1
E. coli Ribosomal Proteins That Stimulate Yeast Flp/Frt Recombinase

| | No. of Basic | | |
|---|---|---|---|
| E. coli | Residues | | |
| Ribosomal | (Percentage of | Total No. of | |
| Protein | Total) | Residues | Molec. Weight |
| S3 | 39 (16.8%) | 232 | 25,852 |
| S4 | 39 (19.2%) | 203 | 23,137 |
| S5 | 22 (13.3%) | 166 | 17,515 |
| L2 | 48 (17.8%) | 269 | 29,416 |

### SUMMARY OF THE INVENTION

The present invention provides compositions and methods for obtaining amplified, chimeric or recombinant nucleic acid molecules using recombinational cloning, in vitro or in vivo. These methods are highly specific, rapid, and less labor intensive than standard cloning or subcloning techniques. The improved specificity, speed and yields of the present invention facilitates DNA or RNA cloning or subcloning, regulation or exchange useful for any related purpose.

In certain aspects, the invention provides compositions comprising at least one (e.g., one, two, three, four, five, six, seven, eight, ten, etc.) recombination protein and at least one (e.g., one, two, three, four, five, six, seven, eight, ten, etc.) Fis protein and/or Fis protein fragment, wherein the recombination protein is present in an amount effective for recombinational cloning of at least one (e.g., one, two, three, four, five, six, seven, eight, ten, etc.) nucleic acid molecule and the Fis protein and/or Fis protein fragment is present in an amount effective for enhancing the efficiency of the recombinational cloning.

In specific embodiments, Fis proteins present in compositions of the invention are not full-length Fis proteins. In related embodiments, compositions of the invention may contain full-length Fis proteins which are not Fis proteins from Escherichia coli (e.g., a Fis protein having the amino acid sequence shown in SEQ ID NO:1).

In related aspects, compositions of the invention may further comprise at least one (e.g., one, two, three, four, five, six, seven, eight, ten, etc.) nucleic acid molecule, which may be a linear nucleic acid molecule, a closed, circular nucleic acid molecule, and/or a vector (e.g., an Insert Donor molecule, a Vector Donor molecule, a Cointegrate molecule, a Product molecule and/or a Byproduct molecule). Further, closed, circular nucleic acid molecules present in compositions of the invention may be supercoiled.

In additional related aspects, compositions of the invention may further comprise at least one (e.g., one, two, three, four, five, six, seven, eight, ten, etc.) ribosomal protein (e.g., a prokaryotic or eukaryotic ribosomal protein) and/or ribosomal protein fragment, wherein the ribosomal protein and/or ribosomal protein fragment is present in an amount effective for enhancing the efficiency of the recombinational cloning. Further, the ribosomal protein may be an Escherichia coli ribosomal protein, such as an E. coli ribosomal protein selected from the group of E. coli ribosomal proteins consisting of S10, S14, S15, S16, S17, S18, S19, S20, S21, L14, L21, L23, L24, L25, L27, L28, L29, L30, L31, L32, L33 and L34. In addition, ribosomal protein(s) included in compositions of the invention may comprise one or more basic ribosomal proteins. Furthermore, ribosomal protein(s) included in compositions of the invention may comprise one or more ribosomal proteins and/or ribosomal protein fragments having a molecular weight of less than about 14 kiloDaltons (kDa).

In other related aspects, compositions of the invention may comprise one or more Fis proteins from an organism selected from the group consisting of Escherichia coli, Salmonella typhimurium, Klebsiella pneumoniae, Vibrio cholera, Haemophilus influenza, and Pseudomonas aeruginosa.

In yet other related aspects, compositions of the invention may comprise one or more Fis proteins comprising amino acid sequences at least 90% identical to an amino acid sequence selected from the group consisting of the amino acid sequence of SEQ ID NO:1, the amino acid sequence of SEQ ID NO:2, the amino acid sequence of SEQ ID NO:3, the amino acid sequence of SEQ ID NO:4, the amino acid sequence of SEQ ID NO:5, and the amino acid sequence of SEQ ID NO:6.

In yet further related aspects, compositions of the invention may comprise one or more Fis protein fragments comprising at least 15 amino acid residues of an amino acid sequence selected from the group consisting of the amino acid sequence of SEQ ID NO:1, the amino acid sequence of SEQ ID NO:2, the amino acid sequence of SEQ ID NO:3, the amino acid sequence of SEQ ID NO:4, the amino acid sequence of SEQ ID NO:5, and the amino acid sequence of SEQ ID NO:6.

In further related aspects, the recombination protein in compositions of the invention are either prokaryotic recombination proteins or one or more recombination proteins selected from the group consisting of Int, Cre, FLP, Xis, IHF and HU, as well as combinations thereof.

In another aspect, the invention provides methods for in vitro cloning of nucleic acids of interest, these methods comprise:

(a) mixing in vitro a first vector comprising at least a first recombination site and a second vector comprising at least a second recombination site, wherein the first and/or second vector further comprises a nucleic acid of interest; and

(b) incubating the mixture in the presence of at least one recombination protein and at least one protein or protein fragment which enhances recombination efficiency under conditions sufficient to cause recombination of at least the first and second recombination sites, thereby producing a chimeric nucleic acid molecule comprising the nucleic acid of interest. Optionally, the mixture of step (b) above, which contains the chimeric nucleic acid molecule, may then be contacted with one or more host cells, followed by selecting for host cells comprising the chimeric nucleic acid molecule, selecting against host cells comprising the first vector and selecting against host cells comprising the second vector, thereby cloning the nucleic acid of interest.

In specific embodiments, the at least one protein or protein fragment which enhances recombination efficiency comprises at least one Fis protein or Fis protein fragment.

The invention also provides in vitro methods for apposing expression signals and open reading frames or partial open reading frames, these methods comprise:

(a) mixing in vitro a first nucleic acid molecule comprising the expression signal and at least a first recombination site, and a second nucleic acid molecule comprising the open reading frame or partial open reading frame and at least a second recombination site; and

(b) incubating the mixture of (a) in vitro in the presence of at least one recombination protein and at least one protein or protein fragment which enhances recombination efficiency under conditions sufficient to cause recombination of at least the first and second recombination site thereby apposing the expression signal and the open reading frame or partial open reading frame such that expression of the open reading frame or partial open reading frame can be controlled by the expression signal.

In related embodiments, the invention provides in vitro methods for apposing expression signals and nucleic acid segments which are expressible but do not comprise open reading frames or a partial open reading frames. Examples of such nucleic acid segments include DNA which encodes tRNA molecules, rRNA molecules, and ribozymes.

In specific embodiments, the at least one protein or protein fragment which enhances recombination efficiency comprises at least one Fis protein or Fis protein fragment.

The invention further provides methods for recombinational cloning of at least one (e.g., one, two, three, four, five, six, seven, eight, ten, etc.) first nucleic acid molecule, the method comprising:

(a) forming a mixture by mixing the first nucleic acid molecule with at least one (e.g., one, two, three, four, five, six, seven, eight, ten, etc.) second nucleic acid molecule and with a composition of the invention described above; and

(b) incubating the mixture formed in (a) under conditions sufficient to recombine the first nucleic acid molecule with the second nucleic acid molecule,

wherein the first nucleic acid molecule and the second nucleic acid molecule each comprise at least one (e.g., one, two, three, four, five, six, seven, eight, ten, etc.) recombination site.

In specific embodiments, the first nucleic acid molecule used in these methods may be either genomic DNA or cDNA. Additionally, the first nucleic acid molecule may be produced by chemical synthesis or by either in vivo or in vitro amplification.

In additional specific embodiments, the second nucleic acid molecule may comprise one or more vectors. Examples of such vectors include vectors capable of replicating in prokaryotic cells (e.g., vectors capable of replicating in bacteria of the genera Escherichia, Salmonella, Bacillus, Streptomyces, and/or Pseudomonas), eukaryotic cells (e.g., vectors capable of replicating in yeast cells, plant cells, fish cells, mammalian cells, and/or insect cells), or both prokaryotic and eukaryotic cells.

The invention also provides methods for enhancing the efficiency of recombinational cloning reactions. These methods comprise contacting at least two (e.g., two, three, four, five, six, seven, eight, ten, etc.) nucleic acid molecules with (1) at least one (e.g., one, two, three, four, five, six, seven, eight, ten, etc.) Fis protein and/or Fis protein fragment and (2) at least one (e.g., one, two, three, four, five, six, seven, eight, ten, etc.) recombination protein, wherein the nucleic acid molecules comprise at least one (e.g., one, two, three, four, five, six, seven, eight, ten, etc.) recombination site.

In specific embodiments, methods of the invention further include the use of compositions which comprise at least one (e.g., one, two, three, four, five, six, seven, eight, ten, etc.) ribosomal protein (e.g., a prokaryotic or eukaryotic ribosomal protein) and/or ribosomal protein fragment, wherein the ribosomal protein and/or ribosomal protein fragment is present in an amount effective for enhancing the efficiency of the recombinational cloning, as well as additional compositions described above and elsewhere wherein.

The invention further provides methods for cloning at least one (e.g., one, two, three, four, five, six, seven, eight, ten, etc.) nucleic acid molecule comprising a nucleic acid segment flanked by at least two (e.g., two, three, four, five, six, seven, eight, ten, etc.) recombination sites, wherein the recombination sites do not substantially recombine with each other. These method comprise:

(a) forming a combination by combining in vitro or in vivo:

(i) at least one (e.g., one, two, three, four, five, six, seven, eight, ten, etc.) Insert Donor molecule comprising the nucleic acid molecule;

(ii) at least one (e.g., one, two, three, four, five, six, seven, eight, ten, etc.) first Vector Donor molecule comprising at least two (e.g., two, three, four, five, six, seven, eight, ten, etc.) recombination sites, wherein the recombination sites do not substantially recombine with each other;

(iii) an effective amount of at least one (e.g., one, two, three, four, five, six, seven, eight, ten, etc.) recombination protein; and

(iv) an effective amount of at least one (e.g., one, two, three, four, five, six, seven, eight, ten, etc.) Fis protein and/or Fis protein fragment; and

(b) incubating the combination under conditions sufficient to transfer the nucleic acid molecule(s) into the first Vector Donor molecule(s), thereby producing at least one (e.g., one, two, three, four, five, six, seven, eight, ten, etc.) first Product molecule.

The methods described directly above may further comprise:

(c) forming a combination by combining in vitro or in vivo:

(i) the first Product molecule(s) comprising the nucleic acid molecule;

(ii) at least one (e.g., one, two, three, four, five, six, seven, eight, ten, etc.) second Vector Donor molecule comprising two or more (e.g., two, three, four, five, six, seven, eight, ten, etc.) recombination sites, wherein the recombination sites do not substantially recombine with each other;

(iii) an effective amount of at least one (e.g., one, two, three, four, five, six, seven, eight, ten, etc.) recombination protein; and

(iv) an effective amount of at least one (e.g., one, two, three, four, five, six, seven, eight, ten, etc.) Fis protein and/or Fis protein fragment; and

(d) incubating the combination under conditions sufficient to transfer the nucleic acid molecule(s) into the second Vector Donor molecule(s), thereby producing at least one (e.g., one, two, three, four, five, six, seven, eight, ten, etc.) second Product molecule(s).

In addition, the combination formed in step (a) may further comprise at least one (e.g., one, two, three, four, five, six, seven, eight, ten, etc.) ribosomal protein and/or ribosomal protein fragment.

In specific embodiments, the invention does not include recombination cloning methods involving recombination reactions between (1) nucleic acid molecules which contain attL and attR sites, (2) supercoiled nucleic acid molecules which contain attL sites and linear nucleic acid molecules which contain attR sites, (3) nucleic acid molecules which each contain a single recombination site, and/or (4) nucleic acid molecules which each contain a single recombination site, wherein the single recombination sites are attL and attR sites.

In additional specific embodiments, the invention does includes recombination cloning methods involving recombination reactions between (1) nucleic acid molecules which contain attB and attP sites, (2) supercoiled nucleic acid molecules and linear nucleic acid molecules which each contain at least one recombination site, wherein the recombination sites on the linear and supercoiled nucleic acid molecules are capable of recombining with each other, (3) supercoiled nucleic acid molecules and supercoiled nucleic acid molecules which each contain at least one recombination site, wherein the recombination sites on the linear and supercoiled nucleic acid molecules are capable of recombining with each other, (4) linear nucleic acid molecules and linear nucleic acid molecules which each contain at least one recombination site, wherein the recombination sites on the linear and supercoiled nucleic acid molecules are capable of recombining with each other, (5) nucleic acid molecules which each contain a least two recombination sites (e.g., at least two recombination sites which are each capable of recombining with recombination sites on at least one other nucleic acid molecule), and/or (6) nucleic acid molecules which each contain a single recombination site, wherein the single recombination sites are attB and attP sites. Any of the above recombination methods may be performed either in vivo or in vitro.

The invention also provides kits for use in recombinational cloning of nucleic acid molecules. These kits comprise at least one (e.g., one, two, three, four, five, six, seven, eight, ten, etc.) Fis protein and/or Fis protein fragment.

In specific embodiments, the above kits further comprise at least one (e.g., one, two, three, four, five, six, seven, eight, ten, etc.) component selected from the group consisting of (a) at least one (e.g., one, two, three, four, five, six, seven, eight, ten, etc.) nucleic acid molecule, (b) at least one (e.g., one, two, three, four, five, six, seven, eight, ten, etc.) recombination protein and/or compositions comprising at least one recombination protein, (c) at least one (e.g., one, two, three, four, five, six, seven, eight, ten, etc.) enzyme having ligase activity, (d) at least one (e.g., one, two, three, four, five, six, seven, eight, ten, etc.) enzyme having polymerase activity, (e) at least one (e.g., one, two, three, four, five, six, seven, eight, ten, etc.) enzyme having reverse transcriptase activity, (f) at least one (e.g., one, two, three, four, five, six, seven, eight, ten, etc.) enzyme having restriction endonuclease activity, (g) at least one (e.g., one, two, three, four, five, six, seven, eight, ten, etc.) ribosomal protein and/or ribosomal protein fragment, (h) at least one (e.g., one, two, three, four, five, six, seven, eight, ten, etc.) primer, (i) at least one (e.g., one, two, three, four, five, six, seven, eight, ten, etc.) buffer, (j) at least one (e.g., one, two, three, four, five, six, seven, eight, ten, etc.) transfection reagent, (k) at least one (e.g., one, two, three, four, five, six, seven, eight, ten, etc.) host cell, (l) at least one (e.g., one, two, three, four, five, six, seven, eight, ten, etc.) recombination protein, and (m) instructions for using the kit components.

In additional specific embodiments, recombination protein(s) and/or composition comprising recombination protein(s) in kits of the invention are capable of catalyzing recombination between att sites.

In other specific embodiments, recombination protein(s) and/or composition comprising recombination protein(s) in kits of the invention are capable of catalyzing a reaction between attB and attP sites (i.e., a BP reaction), a reaction between attL and attR sites (i.e., an LR reaction), or both BP and LR reactions. Reactions of these types are described in the GATEWAY™ Cloning Technology Instruction Manual (Invitrogen Corp., Life Technologies Division) (see www.lifetech.com/Content/Tech-Online/molecular_biology/manuals_pps/gatewayman.pdf, visited on Mar. 29, 2001), the entire disclosure of which is incorporated herein by reference.

Other embodiments of the present invention will be apparent to one of ordinary skill in light of what is known in the art, the following drawings and description of the invention, and the claims.

### BRIEF DESCRIPTION OF THE DRAWINGS/FIGURES

FIG. 1 depicts one general method of the present invention, wherein the starting (parent) DNA molecules can be circular or linear. The goal is to exchange the new subcloning vector D for the original cloning vector B. It is desirable in one embodiment to select for AD and against all the other molecules, including the Cointegrate. The square and circle are sites of recombination: e.g., loxP sites, att sites, etc. For example, segment D can contain expression signals, new drug markers, new origins of replication, or specialized functions for mapping or sequencing DNA.

FIG. 2 depicts a restriction map for plasmid pHN894. AttP: attP attachment site; 'tet: truncated tetracycline resistance gene; amp: β-lactamase gene.

FIG. 3 depicts a restriction map for plasmid pBB105. AttB: attB attachment site; 'tet: truncated tetracycline resistance gene; amp: β-lactamase gene; ori: colE1 origin of replication; ROP: replication control site.

FIG. 4 depicts a restriction map for plasmid pHN872. AttL: attL attachment site; 'tet: truncated tetracycline resistance gene; 'amp: truncated β-lactamase gene; ori: colE1 origin of replication; KmR: kanamycin resistance gene.

FIG. 5 depicts a restriction map for plasmid pHN868. AttR: attR attachment site; 'tet: truncated tetracycline resistance gene; amp: β-lactamase gene; ori: colE1 origin of replication; ROP: replication control site.

FIG. 6 depicts a restriction map for plasmid pEZ13835. WTattP1: modified attP attachment site; WTattP3: modified attP attachment site; T1T2: transcription terminators; KmR: kanamycin resistance gene; CmR: chloramphenicol resistance gene; ccdB: death gene; ori: colE1 origin of replication.

FIG. 7 depicts a restriction map for plasmid pEZC7501. AttB1: modified attB attachment site; attB3: modified attB attachment site; GFP: truncated green fluorescent protein gene; T7 P: T7 promoter; SP6 P: SP6 promoter; CMV P: CMV promoter; lacI′: lacI promoter; lox p: cre recombination site; small t & poly A: SV40 small tumor antigen intron and poly A signal; f1: f1 intergenic region; incA: phage P1 incompatibility locus; Amp: β-lactamase gene; ori: colE1 origin of replication.

FIG. 8 depicts a restriction map for plasmid pEZ11104. AttL1: modified attL attachment site; attL3: modified attL attachment site; CmR: chloramphenicol resistance gene; KmR: kanamycin resistance gene; ori: colE1 origin of replication.

FIG. 9 depicts a restriction map for plasmid pEZC8402. attR′1: modified attR attachment site; attR′3: modified attR attachment site; lac I: lac repressor gene; amp: β-lactamase gene; ori: colE1 origin of replication; CmR: chloramphenicol resistance gene; f1: f1 intergenic region; ccdB: death gene.

FIG. 10 depicts a restriction map for plasmid pTRCN2. Ap: β-lactamase gene; ptrc: trc promoter; laqI^{Q}: lac repressor gene; f1′ori: f1 intergenic region; ori: colE1 origin of replication.

FIG. 11 depicts a restriction map for plasmid pTRCN2INT2. Ap: β-lactamase gene; ptrc: trc promoter; laqI^{Q}: lac repressor gene; f1′ori: f1 intergenic region; ori: colE1 origin of replication; Int: λ integrase gene.

FIG. 12 depicts a restriction map for plasmid pTRCN2XIS1. Ap: β-lactamase gene; ptrc: trc promoter; laqI^{Q}: lac repressor gene; f1′ori: f1 intergenic region; ori: colE1 origin of replication; xis: λ xis gene.

FIG. 13 depicts a restriction map for plasmid pTRCN2S20AA. Ap: β-lactamase gene; ptrc: trc promoter; laqI^{Q}: lac repressor gene; f1′ori: f1 intergenic region; ori: colE1 origin of replication; rpsT: S20 gene.

FIG. 14 depicts a restriction map for plasmid pET12AS20AA. Ap: β-lactamase gene; ori: colE1 origin of replication; 'rpsT: S20 gene; T7: T7 promoter; T7 term: T7 transcription termination sequence.

FIG. 15 is a photograph of an SDS-PAGE gel of fractions from phosphocellulose column fractionation of proteins not bound by hydroxyapatite. Aliquots (7.5 μl) from fractions 13 through 20 of the phosphocellulose column of proteins not bound by hydroxyapatite were analyzed by SDS PAGE. IHF (“IHF A”: 0.3 μg; “IHF B”: 0.5 μg) and BenchMark protein standards (“M”) were run as references. The bottom of the figure indicates the relative ability of aliquots from the fractions to stimulate Int in an integrative recombination gel assay (−, no stimulation; +, ++, +++, increasing levels of stimulation).

FIG. 16 is a photograph of an SDS-PAGE gel of S20 ribosomal protein purified from a side fraction of a native Int purification. Lanes M: BenchMark protein standards; lanes A through E: 5 μl, 2 μl, 2 μl, 1 μl, and 1 μl aliquots, respectively, of Mono S pool of S20.

FIG. 17 is a photograph of an ethidium bromide-stained gel in an integrative recombination gel assay (see Materials and Methods) showing the ability of S20 protein in the Mono S pool (see FIG. 16) to stimulate Int activity. Lane A: Int plus S20; lane B: Int alone; lane C: Int dilution buffer alone. The slowest migrating band is the recombinant DNA product.

FIG. 18 is a photograph of an SDS-PAGE gel of peak fractions containing integrative recombination stimulatory activity from the Mono S columns described in Materials and Methods section Purification of Stimulatory Proteins from Cells producing Native Int and Results section PART II: Purification and Identification of the Stimulatory Proteins. Phosphocellulose Pool #1 was fractionated on a Mono S column producing two peaks of activity at fraction 18 (1 μl and 2 μl, lanes A and B) and fraction 22 (1 μl and 2 μl, lanes C and D). Phosphocellulose Pool #2 was fractionated in a second run on the same Mono S column producing one peak of activity at fraction 24 (1 μl and 2 μl, lanes F and G). S20 was run in lane E and BenchMark protein standard in lane M.

FIG. 19 is a photograph of an ethidium bromide-stained gel in an integrative recombination gel assay (Materials and Methods) showing stimulation of 37 ng of native Int by 900 ng of recombinant S20 (FIG. 19), 900 ng of S20 (see FIG. 16), and 10 μg of L27 (fraction 18 in FIG. 18). Lane A: recombinant S20; lane B: S20; lane C: L27; lane D: Int alone; lane E: no added Int or stimulatory protein.

FIG. 20 is a photograph of an SDS-PAGE gel of 2 μg of purified recombinant S20.

FIG. 21 is a photograph of an ethidium bromide-stained gel in integrative (lanes A to C) and excisive (lanes D to F) recombination gel assays, showing the recombinase activity of 59 ng of Int-His₆ in the presence of 0 ng (lanes B and E) and 382 ng (lanes C and F) of recombinant S20. All assays also contained 12.5 ng IHF. Excisive recombination assays contained 42 ng Xis-His₆. The assays analyzed in lanes A and D contained no Int-His₆ or rS20.

FIG. 22 shows experiments related to Fis stimulation of single-site LR recombination reactions. Reactions (20 μl) were performed using 100 fmol pATTL2 and 100 fmol pATTR2-BamHI substrates (see “Experimental Methods” in Example 3 below). The percentage of recombination product observed at given Fis concentrations is plotted for three different concentrations of Xis. Percent product was determined by dividing the amount of radioactivity in the product band by the sum of the amount of radioactivity in the substrate and product bands.

FIG. 23 shows experiments related to Fis stimulation of double-site BP recombination reactions. Reactions (20 μl) were performed using 100 fmol pDONR201 (Invitrogen Corp., Life Technologies Division (Rockville, Md.), Catalog No. 11798-014) and 100 fmol pBGFP2-XhoI substrates (see “Experimental Methods” in Example 3 below). The percentage of recombination product observed at given Fis concentrations is plotted for two different concentrations of NaCl. Percent product was determined by dividing the amount of radioactivity in the product band by the sum of the amount of radioactivity in the substrate, cointegrate, and product bands.

FIG. 24 shows experiments related to the effect of salt concentration on Fis stimulation of double-site BP recombination reactions. Reactions (20 μl) were performed using 100 fmol pDONR201 and 100 fmol pBGFP2-XhoI substrates (see “Experimental Methods” in Example 3 below). The percentage of recombination product observed at given NaCl concentrations is plotted for four different concentrations of Fis. Data shown are averages of 3 experiments, with standard deviation shown by error bars.

FIG. 25 shows experiments which indicate that Fis does not stimulate single-site BP recombination reactions under standard conditions. Reactions (20 μl) were performed using 100 fmol pATTP2 and 100 fmol pATTB2-HindIII substrates (see “Experimental Methods” in Example 3 below). The percentage of recombination product observed at given Fis concentrations is plotted for two different salt concentrations. Data shown are averages of 2 separate experiments, with standard deviation shown by error bars.

FIG. 26 shows experiments which demonstrate that Fis stimulation of single-site BP recombination reactions is evident at lower Int concentrations. Reactions (20 μl) were performed using 100 fmol pATTP2 and 100 fmol pATTB2-HindIII substrates (see “Experimental Methods” in Example 3 below). The percentage of recombination product observed at given Int concentrations is plotted for three different Fis concentrations.

FIG. 27 shows experiments related to Fis stimulates single-site BP recombination reactions using linear substrates. Reactions (20 μl) were performed using 100 fmol pATTP2-BamHI and 100 fmol pATTB2-HindIII substrates (see “Experimental Methods” in Example 3 below). The percentage of recombination product observed at given NaCl concentrations is plotted in the presence or absence of Fis. Data shown are averages of 3 separate experiments, with standard deviation shown by error bars.

FIG. 28 shows experiments which indicate that Fis stimulates single-site BP recombination reactions using altered topology substrates. Reactions (20 μl) were performed using 100 fmol pATTB2 and 100 fmol pATTP2-BamHI substrates (see “Experimental Methods” in Example 3 below). The percentage of recombination product observed at given NaCl concentrations is plotted in the presence or absence of Fis. Data shown are averages of 3 separate experiments, with standard deviation shown by error bars.

### DETAILED DESCRIPTION OF THE INVENTION

Overview

It has been discovered that Fis proteins may stimulate in vitro and in vivo recombination activity of recombination systems, such as the λ Int recombination system. Thus, the invention provides compositions comprising such Fis proteins, and methods using such compositions, which are useful in performing reversible and/or repeatable cloning and subcloning reactions to manipulate nucleic acid molecules in order to form chimeric nucleic acids using recombination proteins (e.g., λ Int) and recombination sites. Recombinational cloning according to the present invention thus uses compositions comprising one or more (e.g., one, two, three, four, five, six, eight, ten, etc.) Fis proteins, and one or more (e.g., one, two, three, four, five, six, eight, ten, etc.) recombination proteins (which may be site-specific prokaryotic recombination proteins), in combination with recombinant nucleic acid molecules having at least one (e.g., one, two, three, four, five, six, eight, ten, etc.) selected recombination site for moving or exchanging segments of nucleic acid molecules, in vitro and/or in vivo.

Methods of the invention use recombination reactions to generate chimeric DNA or RNA molecules that have desired characteristic(s) and/or nucleic acid segment(s). Methods of the invention function such that one or more nucleic acid molecules of interest may be moved or transferred into or between any number of vector systems. In accordance with the invention, such transfer to or between various vector systems may be accomplished separately, sequentially or in mass (e.g., into any number of different vectors in one step). The improved specificity, speed and/or yields of the present invention facilitates DNA or RNA cloning, subcloning, regulation or exchange useful for any related purpose. Such purposes include in vitro recombination of DNA or RNA segments and in vitro or in vivo insertion or modification of transcribed, replicated, isolated or genomic DNA or RNA.

Definitions

In the description that follows, a number of terms used in recombinant DNA technology are utilized extensively. In order to provide a clear and consistent understanding of the specification and claims, including the scope to be given such terms, the following definitions are provided.

Adapter: is an oligonucleotide or nucleic acid fragment or segment (e.g., DNA) which comprises one or more recombination sites (or portions of such recombination sites) which in accordance with the invention can be added to a circular or linear Insert Donor molecule as well as other nucleic acid molecules described herein. When using portions of recombination sites, the missing portion may be provided by the Insert Donor molecule. Such adapters may be added at any location within a circular or linear molecule, although the adapters may be added at or near one or both termini of a linear molecule. For example, adapters may be positioned to be located on both sides (flanking) a particularly nucleic acid molecule of interest. In accordance with the invention, adapters may be added to nucleic acid molecules of interest by standard recombinant techniques (e.g., restriction digest and ligation). For example, adapters may be added to a circular molecule by first digesting the molecule with an appropriate restriction enzyme, adding the adapter at the cleavage site and reforming the circular molecule which contains the adapter(s) at the site of cleavage. Alternatively, adapters may be ligated directly to one or more termini of a linear molecule or both termini of the molecule thereby resulting in linear molecule(s) having adapters at one or both termini. In one aspect of the invention, adapters may be added to a population of linear molecules, (e.g., a cDNA library or genomic DNA which has been cleaved or digested) to form a population of linear molecules containing adapters at one and/or both termini of all or substantial portion of said population.

Amplification: depending on the context, as used herein, the term “amplification” refers to any in vitro method for increasing the number of copies of a nucleic acid with the use of a polymerase. Nucleic acid amplification results in the incorporation of nucleotides into a DNA and/or RNA molecule or primer thereby forming a new molecule complementary to a template. The formed nucleic acid molecule and its template can be used as templates to synthesize additional nucleic acid molecules. As used herein, one amplification reaction may consist of many rounds of replication. DNA amplification reactions include, for example, polymerase chain reaction (PCR). One PCR reaction may consist of 5-100 “cycles” of denaturation and synthesis of a DNA molecule.

The term “amplification” can also refer to the production of nucleic acid in vivo, which often occurs after introduction into a cell. Thus, a plasmid, for example, may be amplified by transformation of cells in which the plasmid is capable of replicating. These cells may then be cultured and the “amplified” plasmid can then be isolated.

By-product: is a daughter molecule (a new clone produced after the second recombination event during the recombinational cloning process) lacking the segment which is desired to be cloned or subcloned.

Cointegrate: is at least one recombination intermediate nucleic acid molecule of the present invention that contains both parental (starting) molecules. It will usually be circular. In some embodiments it can be linear.

Fis protein: refers to Fis proteins derived from any number of organisms, as well as mutants and derivatives of Fis proteins, which enhance the efficiency of one or more recombination reactions (e.g., a recombination reaction of the λ Int recombination system). Examples of Fis proteins are set out in SEQ ID NOs:1-6.

Fis protein fragment: refers to Fis protein fragments, as well as mutants and derivatives of such fragments, which enhance the efficiency of one or more recombination reactions (e.g., a recombination reaction of the λ Int recombination system). Generally, Fis protein fragments suitable for use in methods of the invention will comprise at least 15 amino acids.

Host: is any prokaryotic or eukaryotic organism that can be a recipient of the recombinational cloning Product. A “host,” as the term is used herein, includes prokaryotic or eukaryotic organisms that can be genetically engineered. For examples of such hosts, see Maniatis et al., Molecular Cloning: A Laboratory Manual, Cold Spring Harbor Laboratory, Cold Spring Harbor, N.Y. (1982).

Hybridization: The terms “hybridization” and “hybridizing” refers to base pairing of two complementary single-stranded nucleic acid molecules (RNA and/or DNA) to give a double stranded molecule. As used herein, two nucleic acid molecules may hybridize, although the base pairing is not completely complementary. Accordingly, mismatched bases do not prevent hybridization of two nucleic acid molecules provided that appropriate conditions, well known in the art, are used.

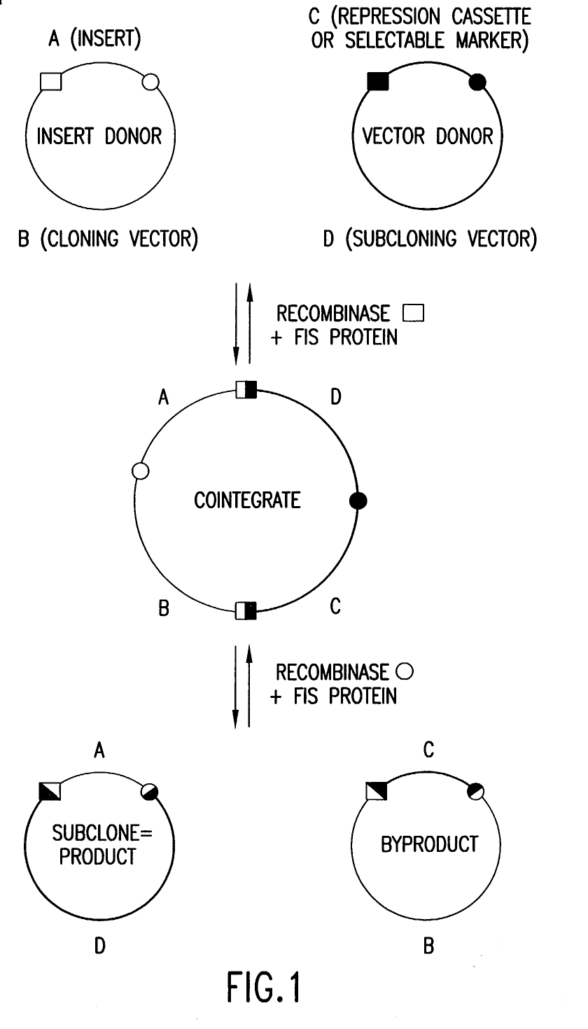

Insert or Inserts: include the desired nucleic acid segment or a population of nucleic acid segments (segment A of FIG. 1) which may be manipulated by the methods of the present invention. Thus, the terms Insert(s) are meant to include a particular nucleic acid (e.g., DNA) segment or a population of segments. Such Insert(s) can comprise one or more genes or open reading frames (ORFs).

Insert Donor: is one of the two parental nucleic acid molecules (e.g., RNA or DNA) of the present invention which carries the Insert. The Insert Donor molecule comprises the Insert flanked on both sides with recombination sites. The Insert Donor can be linear or circular. In one embodiment of the invention, the Insert Donor is a circular DNA molecule and further comprises a cloning vector sequence outside of the recombination signals (see FIG. 1).

When a population of Inserts or population of nucleic acid segments are used to make the Insert Donor, a population of Insert Donors result and may be used in accordance with the invention.

Library: refers to a collection of nucleic acid molecules (circular or linear) which differ in nucleotide sequence (e.g., populations of nucleic acid molecules in which at least 500, 1,000, 2,000, 3,000, 5,000, 10,000, 15,000, 20,000, 30,000, 50,000, 70,000, 80,000, etc. of the individual nucleic acid molecules either comprise different sequences or share no regions of sequence identify which are greater than 100 nucleotides). In one embodiment, a library is representative of all or a portion or a significant portion of the nucleic acid content of an organism (a “genomic” library), or a set of nucleic acid molecules representative of all, a portion or a significant portion (e.g., about 50%, about 60%, about 70%, about 80%, about 90%, about 95%, etc.) of the expressed nucleic acid molecules (a cDNA library or segments derived therefrom) in a cell, tissue, organ or organism. A library may also comprise nucleic acid molecules having random sequences made by de novo synthesis, mutagenesis of one or more nucleic acid molecules, and the like. Such libraries may or may not be contained in one vector or two or more (e.g., two, three, four, five, seven, ten, twelve, fifteen, twenty, thirty, fifty, etc.) different vectors. Libraries used in the practice of the invention may be normalized libraries. Further, these libraries may comprise molecules which are linear or circular.

Nucleotide: refers to a base-sugar-phosphate combination. Nucleotides are monomeric units of a nucleic acid sequence (DNA and RNA). The term nucleotide includes ribonucleoside triphosphatase ATP, UTP, CTG, GTP and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP. The term nucleotide as used herein also refers to dideoxyribonucleoside triphosphates (ddNTPs) and their derivatives. Illustrative examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

Oligonucleotide: refers to a synthetic or natural molecule comprising a covalently linked sequence of nucleotides which are joined by a phosphodiester bond between the 3′ position of the deoxyribose or ribose of one nucleotide and the 5′ position of the deoxyribose or ribose of the adjacent nucleotide.

Primer: refers to a single stranded or double stranded oligonucleotide that is extended by covalent bonding of nucleotide monomers during amplification or polymerization of a nucleic acid molecule (e.g., a DNA molecule). In one aspect, the primer comprises one or more recombination sites or portions of such recombination sites. Portions of recombination sites comprise at least 2 bases, at least 5 bases, at least 10 bases or at least 20 bases of the recombination sites of interest. When using portions of recombination sites, the missing portion of the recombination site may be provided by the newly synthesized nucleic acid molecule. Such recombination sites may be located within and/or at one or both termini of the primer. Further, additional sequences may be added to the primer adjacent to the recombination site(s) to enhance or improve recombination and/or to stabilize the recombination site during recombination. Such stabilization sequences may be any sequences (e.g., G/C rich sequences) of any length. Such sequences may range in size, for example, from 1 to about 1000 bases, 1 to about 500 bases, and 1 to about 100 bases, 1 to about 60 bases, 1 to about 25, 1 to about 10, 2 to about 10 or about 4 bases. In most instances, such sequences are greater than 1 or 2 bases in length.

Product: is one the desired daughter molecules comprising the A and D sequences which is produced after the second recombination event during the recombinational cloning process (see FIG. 1). The Product contains the nucleic acid which was to be cloned or subcloned. In accordance with the invention, when a population of Insert Donors are used, the resulting population of Product molecules will contain all or a portion of the population of Inserts of the Insert Donors and, in many instances, will contain a representative population of the original molecules of the Insert Donors.

Promoter: is a DNA sequence generally described as the 5′-region of a gene, located proximal to the start codon. The transcription of an adjacent DNA segment is initiated at the promoter region. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

Protein which enhances the efficiency of recombination reactions: refers to a protein or peptide which either (1) increases the rate of a recombination reaction or (2) increases the amount of end product resulting from a recombination reaction. Examples of such proteins include Fis proteins and E. coli ribosomal proteins S10, S14, S15, S16, S17, S18, S19, S20, S21, L14, L21, L23, L24, L25, L27, L28, L29, L30, L31, L32, L33 and L34. Further examples are protein fragments (e.g., Fis protein fragments) which enhance the efficiency of recombination reactions. Additional examples are proteins and protein fragments which bind to nucleic acid molecules that Fis binds to (e.g., nucleic acid molecules comprising the nucleotide sequence shown in SEQ ID NO:7 or SEQ ID NO:8) and enhance the efficiency of one or more recombination reactions.

An amount effective for enhancing the efficiency of recombinational cloning: refers to amounts of proteins or protein fragments which enhance the efficiency of recombination reactions. Methods for determining such amounts are set out below in Example 3. In general, proteins or protein fragments which enhance the efficiency of recombination reactions will be included in amounts which result in measurable increases (e.g., increases of at least 5%, at least 10%, at least 15%, at least 20%, at least 25%, at least 30%, at least 35%, at least 50%, etc.) in the efficiency of one or more recombination reactions in comparison to recombination reactions performed in the absence of the proteins or protein fragments. One example of an assay which can be used to measure Fis activity, as well as whether a composition enhances the efficiency of recombination reactions, is the “Recombination assays” section set out below in Example 3.

Recognition sequence: Recognition sequences are particular sequences which a protein, chemical compound, DNA, or RNA molecule (e.g., restriction endonuclease, a modification methylase, or a recombinase) recognizes and binds. In the present invention, a recognition sequence will usually refer to a recombination site. For example, the recognition sequence for Cre recombinase is loxP which is a 34 base pair sequence comprised of two 13 base pair inverted repeats (serving as the recombinase binding sites) flanking an 8 base pair core sequence. See FIG. 1 of Sauer, B., Current Opinion in Biotechnology 5:521-527 (1994). Other examples of recognition sequences are the attB, attP, attL, and attR sequences which are recognized by the recombinase enzyme λ Integrase. AttB is an approximately 25 base pair sequence containing two 9 base pair core-type Int binding sites and a 7 base pair overlap region. AttP is an approximately 240 base pair sequence containing core-type Int binding sites and arm-type Int binding sites as well as sites for auxiliary proteins integration host factor (IHF), Fis, and excisionase (Xis). See Landy, Current Opinion in Biotechnology 3:699-707 (1993). Such sites may also be engineered according to the present invention to enhance production of products in the methods of the invention. When such engineered sites lack the P1 or H1 domains to make the recombination reactions irreversible (e.g., attR or attP), such sites may be designated attR′ or attP′ to show that the domains of these sites have been modified in some way.

Recombinase: is a type of recombination protein which catalyzes the exchange of DNA segments at specific recombination sites.

Recombinational Cloning: is a method described herein, whereby segments of nucleic acid molecules or populations of such molecules are exchanged, inserted, replaced, substituted or modified, in vitro or in vivo.

Recombination proteins: include excisive or integrative proteins, enzymes, co-factors or associated proteins that are involved in recombination reactions involving one or more recombination sites. See Landy, A., Current Opinion in Biotechnology 3:699-707 (1993).

Repression cassette: is a nucleic acid segment that contains a repressor of a Selectable marker present in the subcloning vector.

Ribosomal protein: is a protein, or a mutant or derivative thereof, that is a constituent of a subunit of a ribosome. According to the invention, the ribosome may be a prokaryotic or eukaryotic ribosome. One example of a ribosome is an E. coli ribosome, which comprises a 30S and a 50S subunit.

Ribosomal protein fragment: is a fragment of a protein that is a constituent of a subunit of a ribosome. Generally, ribosomal protein fragments used in the practice of the invention will be functional fragments. By a “functional” fragment is meant a fragment of a native ribosomal protein, or a mutant or derivative of such a fragment, that has substantially the same biological activity as the corresponding native ribosomal protein in stimulating one or more recombination reactions (e.g., a recombination reaction of the λ Int recombination system).

Selectable marker: is a DNA segment that allows one to select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like. Examples of Selectable markers include but are not limited to: (1) DNA segments that encode products which provide resistance against otherwise toxic compounds (e.g., antibiotics); (2) DNA segments that encode products which are otherwise lacking in the recipient cell (e.g., tRNA genes, auxotrophic markers); (3) DNA segments that encode products which suppress the activity of a gene product; (4) DNA segments that encode products which can be readily identified (e.g., phenotypic markers such as β-galactosidase, green fluorescent protein (GFP), and cell surface proteins); (5) DNA segments that bind products which are otherwise detrimental to cell survival and/or function; (6) DNA segments that otherwise inhibit the activity of any of the DNA segments described in Nos. 1-5 above (e.g., antisense oligonucleotides); (7) DNA segments that bind products that modify a substrate (e.g., restriction endonucleases); (8) DNA segments that can be used to isolate or identify a desired molecule (e.g., specific protein binding sites); (9) DNA segments that encode a specific nucleotide sequence which can be otherwise non-functional (e.g., for PCR amplification of subpopulations of molecules); (10) DNA segments, which when absent, directly or indirectly confer resistance or sensitivity to particular compounds; and/or (11) DNA segments that encode products which are toxic in recipient cells.

Selection scheme: is any method which allows selection, enrichment, or identification of a desired Product or Product(s) from a mixture containing the Insert Donor, Vector Donor, any intermediates (e.g., a Cointegrate), and/or Byproducts. The selection schemes of one embodiment have at least two components that are either linked or unlinked during recombinational cloning. One component is a Selectable marker. The other component controls the expression in vitro or in vivo of the Selectable marker, or survival of the cell harboring the plasmid carrying the Selectable marker. Generally, this controlling element will be a repressor or inducer of the Selectable marker, but other means for controlling expression of the Selectable marker can be used. Whether a repressor or activator is used will depend on whether the marker is for a positive or negative selection, and the exact arrangement of the various DNA segments, as will be readily apparent to those skilled in the art. In many instances, the selection scheme will result in selection of or enrichment for only one or more desired Products. As defined herein, selecting for a DNA molecule includes (a) selecting or enriching for the presence of the desired DNA molecule, and (b) selecting or enriching against the presence of DNA molecules that are not the desired DNA molecule.

In one embodiment, the selection schemes (which can be carried out in reverse) will take one of three forms, which will be discussed in terms of FIG. 1. The first, exemplified herein with a Selectable marker and a repressor therefore, selects for molecules having segment D and lacking segment C. The second selects against molecules having segment C and for molecules having segment D. Possible embodiments of the second form would have a DNA segment carrying a gene toxic to cells into which the in vitro reaction products are to be introduced. A toxic gene can be a DNA that is expressed as a toxic gene product (a toxic protein or RNA), or can be toxic in and of itself. (In the latter case, the toxic gene is understood to carry its classical definition of “heritable trait”.)

Examples of such toxic gene products are well known in the art, and include, but are not limited to, apoptosis-related genes (e.g., ASKi or members of the bcl-2/ced-9 family), retroviral genes including those of the human immunodeficiency virus (HIV), defensins such as NP-1, inverted repeats or paired palindromic DNA sequences, bacteriophage lytic genes such as those from φX174 or bacteriophage T4; antibiotic sensitivity genes such as rpsL, antimicrobial sensitivity genes such as pheS, plasmid killer genes, eukaryotic transcriptional vector genes that produce a gene product toxic to bacteria, such as GATA-1, and genes that kill hosts in the absence of a suppressing function, e.g., kicB or ccdB. A toxic gene can alternatively be selectable in vitro, e.g., a restriction site.

In the second form, segment D carries a Selectable marker. The toxic gene would eliminate transformants harboring the Vector Donor, Cointegrate, and Byproduct molecules, while the Selectable marker can be used to select for cells containing the Product and against cells harboring only the Insert Donor.

The third form selects for cells that have both segments A and D in cis on the same molecule, but not for cells that have both segments in trans on different molecules. This could be embodied by a Selectable marker that is split into two inactive fragments, one each on segments A and D.

The fragments are so arranged relative to the recombination sites that when the segments are brought together by the recombination event, they reconstitute a functional Selectable marker. For example, the recombinational event can link a promoter with a structural gene, can link two fragments of a structural gene, or can link genes that encode a heterodimeric gene product needed for survival, or can link portions of a replicon.

Site-specific recombinase: is a type of recombinase which typically has at least the following four activities (or combinations thereof): (1) recognition of one or two specific nucleic acid sequences; (2) cleavage of said sequence or sequences; (3) topoisomerase activity involved in strand exchange; and (4) ligase activity to reseal the cleaved strands of nucleic acid. See Sauer, B., Current Opinions in Biotechnology 5:521-527 (1994). The strand exchange mechanism involves the cleavage and rejoining of specific DNA sequences in the absence of DNA synthesis (Landy, A. (1989) Ann. Rev. Biochem. 58:913-949).

Subcloning vector: is a cloning vector comprising a circular or linear nucleic acid molecule which often includes an appropriate replicon. In the present invention, the subcloning vector (segment D in FIG. 1) can also contain functional and/or regulatory elements that are desired to be incorporated into the final product to act upon or with the cloned DNA Insert (segment A in FIG. 1). The subcloning vector can also contain a Selectable marker (e.g., DNA).

Template: refers to double stranded or single stranded nucleic acid molecules which are to be amplified, synthesized or sequenced. In the case of double stranded molecules, denaturation of its strands to form a first and a second strand may be performed before these molecules will be amplified, synthesized or sequenced, or the double stranded molecule may be used directly as a template. For single stranded templates, a primer complementary to a portion of the template is hybridized under appropriate conditions and one or more polypeptides having polymerase activity (e.g., DNA polymerases and/or reverse transcriptases) may then synthesize a nucleic acid molecule complementary to all or a portion of said template. Alternatively, for double stranded templates, one or more promoters may be used in combination with one or more polymerases to make nucleic acid molecules complementary to all or a portion of the template. The newly synthesized molecules, according to the invention, may be equal or shorter in length than the original template. Additionally, a population of nucleic acid templates may be used during synthesis or amplification to produce a population of nucleic acid molecules typically representative of the original template population.

Vector: is a nucleic acid molecule (e.g., DNA) that provides one or more biological or biochemical property to an Insert. Examples include plasmids, phages, autonomously replicating sequences (ARS), centromeres, and other sequences which are able to replicate or be replicated in vitro or in a host cell, or to convey a desired nucleic acid segment to a desired location within a host cell. A Vector can have one or more restriction endonuclease recognition sites at which the sequences can be cut in a determinable fashion without loss of an essential biological function of the vector, and into which a nucleic acid fragment can be spliced in order to bring about its replication and cloning. Vectors can further provide primer sites, e.g., for PCR, transcriptional and/or translational initiation and/or regulation sites, recombinational signals, replicons, Selectable markers, etc. Clearly, methods of inserting a desired nucleic acid fragment which do not require the use of homologous recombination, transpositions or restriction enzymes (such as, but not limited to, UDG cloning of PCR fragments (U.S. Pat. No. 5,334,575, entirely incorporated herein by reference), T:A cloning, and the like) can also be applied to clone a fragment into a cloning vector to be used according to the present invention. The cloning vector can further contain one or more selectable markers suitable for use in the identification of cells transformed with the cloning vector.

Vector Donor: is one of the two parental nucleic acid molecules (e.g., RNA or DNA) of the present invention which carries the DNA segments comprising the DNA vector which is to become part of the desired Product. The Vector Donor comprises a subcloning vector D (or it can be called the cloning vector if the Insert Donor does not already contain a cloning vector) and a segment C flanked by recombination sites (see FIG. 1). Segments C and/or D can contain elements that contribute to selection for the desired Product daughter molecule, as described above for selection schemes. The recombination signals can be the same or different, and can be acted upon by the same or different recombinases. In addition, the Vector Donor can be linear or circular.

Other terms used in the fields of recombinant DNA technology and molecular and cell biology as used herein will be generally understood by one of ordinary skill in the applicable arts.

Recombination Schemes

One general scheme for in vitro and/or in vivo methods of the invention is shown in FIG. 1, where the Insert Donor and the Vector Donor can be either circular or linear DNA, but is shown as circular. Vector D is exchanged for the original cloning vector B. The Insert Donor need not comprise a vector. The method of the invention allows the Inserts A to be transferred into any number of vectors. According to the invention, the Inserts may be transferred to a particular Vector or may be transferred to a number of vectors in one step. Additionally, the Inserts may be transferred to any number of vectors sequentially, for example, by using the Product DNA molecule as the Insert Donor in combination with a different Vector Donor. The nucleic acid molecule of interest may be transferred into a new vector thereby producing a new Product DNA molecule. The new Product DNA molecule may then be used as starting material to transfer the nucleic acid molecule of interest into a new vector. Such sequential transfers can be performed a number of times in any number of different vectors. Thus the invention allows for cloning or subcloning nucleic acid molecules and because of the ease and simplicity, these methods are particularly suited for high through-put applications. In accordance with the invention, it is desirable to select for the daughter molecule containing elements A and D and against other molecules, including one or more Cointegrate(s). The square and circle are different sets of recombination sites (e.g., lox sites or att sites). Segment A or D can contain at least one Selection Marker, expression signals, origins of replication, or specialized functions for detecting, selecting, expressing, mapping or sequencing DNA, where D is used in this example. This scheme can also be reversed according to the present invention, as described herein. The resulting product of the reverse reaction (e.g., the Insert Donor) may then be used in combination with one or a number of vectors to produce new product molecules in which the Inserts are contained by any number of vectors.

Examples of desired DNA segments that can be part of Element A or D include, but are not limited to, PCR products, large DNA segments, genomic clones or fragments, cDNA clones or fragments, functional elements, etc., and genes or partial genes, which encode useful nucleic acids or proteins. Moreover, the recombinational cloning of the present invention can be used to make ex vivo and in vivo gene transfer vehicles for protein expression (native or fusion proteins) and/or gene therapy.

In FIG. 1, the scheme provides the desired Product as containing A and Vector D, as follows. The Insert Donor (containing A and B) is first recombined at the square recombination sites by recombination proteins, with the Vector Donor (containing C and D), to form a Cointegrate having each of A-D-C-B. Next, recombination occurs at the circle recombination sites to form Product DNA (A and D) and Byproduct DNA C and B). However, if desired, two or more different Cointegrates can be formed to generate two or more Products.

Recombinational cloning using nucleic acid molecules comprising engineered recombination sites, and the materials and methods by which this technique may be accomplished, have been described in detail in U.S. application Ser. No. 08/486,139, filed Jun. 7, 1995; U.S. application Ser. No. 08/663,002, filed Jun. 7, 1996 (now U.S. Pat. No. 5,888,732); U.S. application Ser. No. 09/005,476, filed Jan. 12, 1998 (now U.S. Pat. No. 6,171,861); U.S. Application No. 60/065,930, filed Oct. 24, 1997; U.S. application Ser. No. 09/177,387, filed Oct. 23, 1998; U.S. Application No. 60/122,389, filed Mar. 2, 1999; U.S. Application No. 60/122,392, filed Mar. 22, 1999; U.S. Application No. 60/126,049, filed Mar. 23, 1999; U.S. application Ser. No. 09/233,493 (now U.S. Pat. No. 6,143,557); U.S. application Ser. No. 09/438,358, filed Nov. 12, 1999; U.S. Application No. 60/136,744, filed May 28, 1999; U.S. application Ser. No. 09/432,085, filed Nov. 2, 1999; U.S. application Ser. No. 09/498,074, filed Feb. 4, 2000; U.S. Application No. 60/108,324, filed Nov. 13, 1998; U.S. application Ser. No. 09/438,358, filed Nov. 12, 1999; U.S. application Ser. No. 09/517,466, filed Mar. 2, 2000; U.S. application Ser. No. 09/732,914, filed Dec. 11, 2000; and PCT Publication No. WO 00/52027, the disclosures of all of which are incorporated herein by reference in their entireties. Recombinational cloning using nucleic acid molecules comprising engineered recombination sites, and the materials and methods by which this technique may be accomplished, have also been described in GATEWAY™ Cloning Technology Instruction Manual (Invitrogen Corp., Life Technologies Division) (see www.lifetech.com/Content/Tech-Online/molecular_biology/manuals_pps/gatewayman.pdf, visited on Mar. 29, 2001) and “FOCUS: An Introduction to GATEWAY Cloning Technology” vol. 23.1, page 4 (see www.lifetech.com/Content/Focus/231004.pdf, visited on Mar. 29, 2001), the entire disclosures of which are incorporated herein by reference.

Compositions

By the present invention, compositions are provided that may be used in recombinational cloning of nucleic acid molecules or segments thereof. Compositions of the invention may comprise mixtures of (1) at least one (e.g., one, two, three, four five, six, eight, ten, etc.) Fis protein or Fis protein fragment, (2) at least one other component used in recombination reactions described herein (e.g., compositions of the invention may include at least one (e.g., one, two, three, four five, six, eight, ten, etc.) recombination protein), (3) at least one nucleic acid molecule comprising at least one recombination site (which may be a vector, a Vector Donor, an insert, an Insert Donor, a Product molecule, intermediates and the like, or combinations thereof). Composition of the invention may further comprise at least one (e.g., one, two, three, four five, six, eight, ten, etc.) ribosomal protein or ribosomal protein fragment. In general, these compositions will be suitable for use in the recombinational cloning of nucleic acid molecules. Further, in many instances, these compositions will result in an increase in the efficiency of recombination reactions, as compared to recombination reactions which performed in the absence of (1) the Fis protein(s) or Fis protein fragment(s) and/or (2) ribosomal protein(s) or ribosomal protein fragment(s).

In related embodiments, the compositions may further comprise one or more additional components, such as one or more (e.g., one, two, three, four five, six, eight, ten, twenty, thirty, fifty, one hundred, one thousand, five thousand, twenty thousand one hundred thousand, etc.) nucleic acid molecules which may be the same or different (including, but not limited to, one or more Insert Donor molecules, one or more Vector Donor molecules, one or more Cointegrate molecules, one or more Product molecules and one or more Byproduct molecules), one or more (e.g., one, two, three, four five, six, eight, ten, etc.) buffer salts, and/or other reagents which may be used in recombinational cloning of nucleic acid molecules.

In related aspects, the Fis proteins, Fis protein fragments, recombination proteins, and/or compositions of the invention may contain one or more (e.g., one, two, three, four five, six, eight, ten, etc.) stabilizing compounds (e.g., glycerol, serum albumin or gelatin) that are traditionally included in stock reagent solutions. Suitable amounts of such stabilizing compounds will be familiar to one of ordinary skill in the art, or may be easily determined using only routine experimentation. For example, glycerol may be used in the compositions of the invention at a concentration (vol/vol) of about 5%-75%, about 10%-65%, about 15%-60%, about 20%-55%, about 25%-50%, or about 50%. In an additional related aspect, the invention provides these compositions in ready-to-use concentrations, obviating the time-consuming dilution and pre-mixing steps necessary with previously available solutions.

Fis Proteins

The one or more (e.g., one, two, three, four five, six, eight, ten, etc.) Fis proteins or Fis protein fragments used in compositions and/or methods of the invention may be obtained from a wide variety of organisms (e.g., bacteria including, but not limited to, those of the genera Escherichia, Serratia, Salmonella, Pseudomonas, Haemophilus, Bacillus, Streptomyces, Staphylococcus, Streptococcus, or other gram positive or gram negative bacteria). Preferred Fis proteins (or portions or fragments thereof) are derived or obtained from prokaryotic organisms.

Generally, Fis proteins and Fis protein fragments used with the invention will have molecular weights which are below 14 kiloDaltons (kDa). Further, in many instances, between about 2% and about 40%, about 5% and about 35%, about 10% and about 35%, about 10% and about 30%, about 15% and about 30%, or about 15% and about 25% of the amino acid residues of these proteins will be basic amino acid residues. By “basic amino acid residues” is meant amino acid residues which have pKₐs above 7.0 (e.g., arginine, lysine, histidine, etc.). Thus, the invention includes compositions which contain the above described Fis proteins and Fis protein fragments, as well as methods for using these compositions in methods of the invention.

One example of a Fis protein is the 98 amino acid Fis protein of E. coli, which has the following amino acid sequence:

| | | |
|---|---|---|
| 1 | MFEQRVNSDV LTVSTVNSQD QVTQKPLRDS VKQALKNYFA QLNGQDVNDL YELVLAEVEQ | (SEQ ID NO:1) |
| 61 | PLLDMVMAYT RGNQTRAALM MGINRGTLRK KLKKYGMN | |

Another example of a Fis protein is the 93 amino acid Fis protein of Klebsiella pneumoniae, which has the following amino acid sequence:

| | | |
|---|---|---|
| 1 | MFEQRVNSDV LTVSTVNSQD QVTQKPLRDS VKQALKNYFA QLNGQDVNDL YELVLAEVEQ | (SEQ ID NO:2) |
| 61 | PLLDMVMQYT RGNQTRAALM MGINRGTLRK KLK | |

Yet another example of a Fis protein is the 98 amino acid Fis protein of Vibrio cholera, which has the following amino acid sequence:

| | | |
|---|---|---|
| 1 | MFEQNLTSEA LTVTTVTSQD QITQKPLRDS VKASLKNYLA QLNGQEVTEL YELVLAEVEQ | (SEQ ID NO:3) |
| 61 | PLLDTIMQYT RGNQTRAATM MGINRGTLRK KLKKYGMN | |

Another example of a Fis protein is the 99 amino acid Fis protein of Haemophilus influenzae, which has the following amino acid sequence:

| | | |
|---|---|---|
| 1 | MLEQQRNSAD ALTVSVLNAQ SQVTSKPLRD SVKQALRNYL AQLDGQDVND LYELVLAEVE | (SEQ ID NO:4) |
| 61 | HFMLDMIMQY TRGNQTRAAN MLGINRGTLR KKLKKYGMG | |

A further example of a Fis protein is the 107 amino acid Fis protein of Pseudomonas aeruginosa, which has the following amino acid sequence:

| | | |
|---|---|---|
| 1 | MTTMTTETLV SGTTPVSDNA NLKQHLTTPT QEGQTLRDSV EKALHNYFAH LEGQPVTDVY | (SEQ ID NO:5) |
| 61 | NMVLCEVEAP LLETVMNHVK GNQTKASELL GLNRGTLRKK LKQYDLL | |

A yet further example of a Fis protein is the 98 amino acid Fis protein of Salmonella typhimurium, which has the following amino acid sequence:

| | | |
|---|---|---|
| 1 | MFEQRVNSDV LTVSTVNSQD QVTQKPLRDS VKQALKNYFA QLNGQDVNDL YELVLAEVEQ | (SEQ ID NO:6) |
| 61 | PLLDMVMQYT RGNQTRAALM MGINRGTLRK KLKKYGMN | |

Methods of the invention employ Fis proteins and Fis protein fragments, as well as variants, derivatives and mutants of Fis proteins and Fis protein fragments which enhance the efficiency of recombination reactions. Fis protein fragments suitable for use with the invention include fragments which comprise at least 10 amino acids, at least 15 amino acids, at least 20 amino acids, at least 30 amino acids, at least 35 amino acids, at least 40 amino acids, at least 45 amino acids, at least 50 amino acids, at least 55 amino acids, at least 60 amino acids, at least 70 amino acids, at least 75 amino acids, at least 80 amino acids, at least 85 amino acids, etc. Fis protein fragments suitable for use with the invention also include fragments which comprise between about 10-20 amino acids, about 20-30 amino acids, about 30-40 amino acids, about 50-60 amino acids, about 60-70 amino acids, about 70-80 amino acids, about 90-100 amino acids, etc.

Proteins which may also be used with the invention include variants, derivatives and mutants which comprise amino acid sequences at least 65%, 70%, 75%, 80%, 85%, 90%, 95%, 98%, or 99% identical to a reference Fis protein (e.g., a Fis protein having an amino acid sequence set out above) or Fis protein fragment.

By a protein or protein fragment having an amino acid sequence at least, for example, 65% “identical” to a reference amino acid sequence is intended that the amino acid sequence of the protein is identical to the reference sequence except that the protein sequence may include up to 35 amino acid alterations per each 100 amino acids of the amino acid sequence of the reference protein. In other words, to obtain a protein having an amino acid sequence at least 65% identical to a reference amino acid sequence, up to 35% of the amino acid residues in the reference sequence may be deleted or substituted with another amino acid, or a number of amino acids up to 35% of the total amino acid residues in the reference sequence may be inserted into the reference sequence. These alterations of the reference sequence may occur at the amino (N—) or carboxy (C—) terminal positions of the reference amino acid sequence or anywhere between those terminal positions, interspersed either individually among residues in the reference sequence or in one or more contiguous groups within the reference sequence. As a practical matter, whether a given amino acid sequence is, for example, at least 65% identical to the amino acid sequence of a reference protein can be determined conventionally using known computer programs such as those described above for nucleic acid sequence identity determinations, or using the CLUSTAL W program (Thompson, J. D., et al., Nucleic Acids Res. 22:4673-4680 (1994)).

Fis protein fragments which may be used in the practice of the invention also comprise N-terminal and C-terminal deletion mutants of Fis proteins (e.g., a Fis protein having an amino acid sequence set out in any of SEQ ID NOs:1-6). Such Fis protein fragments include those in which at least 5 amino acids, at least 10 amino acids, at least 15 amino acids, at least 20 amino acids, at least 25 amino acids, at least 30 amino acids, at least 35 amino acids, at least 40 amino acids, at least 45 amino acids, at least 50 amino acids, at least 55 amino acids, at least 60 amino acids, at least 65 amino acids, at least 70 amino acids, or at least 75 amino acids have been deleted from the N-terminus. Such Fis protein fragments also include those in which at least 1 amino acid, at least 2 amino acids, at least 3 amino acids, at least 4 amino acids, at least 5 amino acids, at least 6 amino acids, at least 7 amino acids, at least 8 amino acids, at least 9 amino acids, or at least 10 amino acids have been deleted from the C-terminus. Further, such Fis protein fragments include proteins comprising both the N-terminal and C-terminal deletions set out above.

Specific examples of Fis deletion mutants which may be used in the practice of the invention include Fis protein fragments comprising amino acids 75-98 of SEQ ID NO:1, amino acid 76-97 of SEQ ID NO:1, amino acid 77-96 of SEQ ID NO:1, amino acid 78-95 of SEQ ID NO:1, amino acid 79-93 of SEQ ID NO:1, or amino acid 80-92 of SEQ ID NO:1, as well as corresponding regions of other Fis proteins.

The invention also includes nucleic acid molecules which encode the Fis proteins referred to herein, as well as the use of these nucleic acid molecules in processes of the invention.

Compositions of the invention may also comprise proteins and protein fragments which bind to nucleic acids that Fis specifically binds to and enhance the efficiency of recombination reactions. For example, Fis has been shown to bind to nucleic acids having the following nucleotide sequence:

GNTYAAWWWTTRANC (SEQ ID NO:7), where R=A or G, W=A or T, and Y=C or T.

Fis also binds to nucleic acids having the following nucleotide sequence:

AGTCTGTTTTTTATGCAAAA (SEQ ID NO:8).

Thus, in certain embodiments, the invention includes methods for enhancing recombination reactions which employ proteins and peptides that (1) bind to nucleic acids having the nucleotide sequence shown in SEQ ID NO:7 or SEQ ID NO:8, or proteins and peptides that bind to nucleic acids having a nucleotide sequence shown in SEQ ID NO:7 or SEQ ID NO:8 (or portions thereof) with one, two, three, or four substitutions, deletions or insertions, and (2) enhance the efficiency of recombination reactions.

Fis proteins and Fis protein fragments of the invention, as well as proteins and peptides which bind nucleic acids that Fis specifically binds to, may be prepared and used as fusion proteins. Fis is believed to form dimers. Thus, examples of fusion proteins which may be used in methods of the invention are fusion proteins which comprises (1) a Fis protein, a Fis protein fragment, or a peptide which binds to nucleic acid comprising the nucleotide sequence shown in SEQ ID NO:7 or SEQ ID NO:8 (or portions thereof) and (2) a protein or protein domain which facilitates the formation of multimers (e.g., homodimers). Examples of such proteins and protein domains include SH2 domains, protein DnaA of Streptomyces, AraC, heat shock protein 90, etc. Thus, the invention includes fusion proteins described above, nucleic acid molecules which encode these fusion proteins, and methods for using these fusion proteins and nucleic acid molecules to enhance the efficiency of recombination reactions.

Fusion proteins of the invention further include fusions where two or more (e.g., two, three, four, etc.) Fis protein, or subportion thereof, are fused together into a single polypeptide chain. These Fis proteins may have identical amino acid sequences or may differ in amino acid sequence. The invention further includes fusion proteins comprising a full-length Fis protein fused to a Fis protein fragment. Examples of Fis protein fragments which can be used to prepare fusion proteins of the invention include proteins comprising amino acids 75-98 of SEQ ID NO:1, amino acid 76-97 of SEQ ID NO:1, amino acid 77-96 of SEQ ID NO:1, amino acid 78-95 of SEQ ID NO:1, amino acid 79-93 of SEQ ID NO:1, or amino acid 80-92 of SEQ ID NO:1, as well as corresponding regions of other Fis proteins.

The invention further includes Fis proteins in which one or more Fis proteins are fused to one or more non-Fis proteins (e.g., glutathione S-transferase (GST), β-glucuronidase (GUS), histidine tags (HIS6), green fluorescent protein (GFP), yellow fluorescent protein (YFP), cyan fluorescent protein (CFP), etc.). Nucleic acid molecules which encode the above Fis fusion proteins are also included within the scope of the invention.

Specific parameters and conditions related to the optimization of recombination reactions performed in the presence of Fis are set out below in Example 3 and can also be determined using known assays. For example, a titration assay may be used to determine the appropriate amount of a purified Fis protein, or the appropriate amount of an extract. Such assays are described in detail in the Examples below.

Fis proteins and Fis protein fragments, as well as other proteins and protein fragments which enhance the efficiency of recombination reactions, may be included in recombination reactions (e.g., BP CLONASE™ or LR CLONASE™ catalyzed recombination reactions) in a variety of concentrations, including about 0.5 ng/μl, about 1.0 ng/μl, about 1.5 ng/μl, about 2.0 ng/μl, about 2.5 ng/μl, about 3.0 ng/μl, about 3.5 ng/μl, about 4.0 ng/μl, about 4.5 ng/μl, about 5.0 ng/μl, about 5.5 ng/μl, about 6.0 ng/μl, about 6.5 ng/μl, about 7.0 ng/μl, about 7.5 ng/μl, about 8.0 ng/μl, about 8.5 ng/μl, about 9.0 ng/μl, about 9.5 ng/μl, about 10.0 ng/μl, about 10.5 ng/μl, about 11.0 ng/μl, about 11.5 ng/μl, about 12.0 ng/μl, about 12.5 ng/μl, about 13.0 ng/μl, about 13.5 ng/μl, about 14.0 ng/μl, about 14.5 ng/μl, about 15.0 ng/μl, about 16.0 ng/μl, about 17.0 ng/μl, about 18.0 ng/μl, about 19.0 ng/μl, about 20.0 ng/μl, about 22.0 ng/μl, about 25.0 ng/μl, about 27.0 ng/μl, about 30.0 ng/μl, about 35.0 ng/μl, or about 40.0 ng/μl. Similarly, Fis may be included in recombination reactions in a variety of ranges, including from about 0.5 ng/μl to about 40.0 ng/μl, from about 0.5 ng/μl to about 30.0 ng/μl, from about 0.5 ng/μl to about 15.0 ng/μl, from about 1.0 ng/μl to about 14.0 ng/μl, from about 5.0 ng/μl to about 10.0 ng/μl, from about 7.0 ng/μl to about 15.0 ng/μl, from about 10.0 ng/μl to about 15.0 ng/μl, from about 5.0 ng/μl to about 30.0 ng/μl, from about 10.0 ng/μl to about 30.0 ng/μl, from about 20 ng/μl to about 30.0 ng/μl, from about 20 ng/μl to about 35.0 ng/μl, or from about 20 ng/μl to about 40.0 ng/μl. Of course, other concentrations and ranges suitable for use in methods of the invention may be determined by one of ordinary skill without undue experimentation by carrying out a titration assay as noted above and as described in detail in the Examples below. Thus, the invention further includes methods described herein which employ proteins that enhance the efficiency of recombination reactions.

Ribosomal Proteins.

The one or more ribosomal proteins or ribosomal protein fragments used in the present compositions may be basic ribosomal proteins. By a “basic” ribosomal protein is meant a ribosomal protein, as well as ribosomal protein fragments, which comprises a relatively high percentage (i.e., ranging from about 15-50%) of basic amino acid residues. In most instances, the ribosomal proteins or ribosomal protein fragments used in compositions and methods of the invention will be no larger than about 14 kiloDaltons (kDa) in size, and often will be about 5 kDa to about 14 kDa, about 6 kDa to about 13 kDa, about 7 kDa to about 12 kDa, or about 8 kDa to about 12 kDa, in size. According to the invention, the one or more ribosomal proteins may be one or more prokaryotic ribosomal proteins (e.g., one or more bacterial ribosomal proteins) or one or more eukaryotic ribosomal proteins (e.g., one or more ribosomal proteins of animals (such as mammals (including humans), fish, birds, reptiles, amphibians, monotremes, and the like), fungi, plants, and the like). In certain compositions, the ribosomal proteins may be one or more prokaryotic ribosomal proteins or ribosomal protein fragments, particularly one or more ribosomal proteins, or fragments of such proteins, obtained from bacteria including, but not limited to, those of the genera Escherichia, Serratia, Salmonella, Pseudomonas, Bacillus, Streptomyces, Staphylococcus, Streptococcus, or other gram positive or gram negative bacteria.

In specific compositions of the invention, the ribosomal proteins may be one or more Escherichia coli ribosomal proteins. For example, E. coli ribosomal proteins suitable for use in the compositions and methods of the invention include, but are not limited to, S10, S14, S15, S16, S17, S18, S19, S20, S21, L14, L21, L23, L24, L25, L27, L28, L29, L30, L31, L32, L33 and L34. Corresponding ribosomal proteins from other sources, including prokaryotic or eukaryotic sources, may be used in accordance with the invention. Such corresponding ribosomal proteins will often correspond (in structure, size, biochemistry, and/or function) to the E. coli ribosomal proteins described herein.

Sources and methods for production and isolation of ribosomal proteins, particularly prokaryotic ribosomal proteins, are described in Example 1 below. In addition, information on sources and isolation of prokaryotic and eukaryotic ribosomal proteins may be found in Ann. Rev. Biochem. 51:155 (1982); Ann. Rev. Biochem. 52:35 (1983); Ann. Rev. Biochem. 53:75 (1984); Ann. Rev. Biochem. 54:507 (1985); Ann. Rev. Biochem. 66:679 (1997); and Bruckner and Cox, Nucl. Acids Res. 17(8):3145-3161 (1989).

The amount of one or more ribosomal proteins which is optimal for use in the compositions and methods of the present invention to drive the recombination reaction can be determined using known assays. Specifically, a titration assay may be used to determine the appropriate amount of a purified ribosomal protein, or the appropriate amount of an extract. Such assays are described in detail in the Examples below. In certain embodiments, for example, the compositions may comprise an effective amount of the E. coli ribosomal proteins S20 or S15, or fragments thereof, for example at a concentration range of about 1 ng/20 μl to about 2500 ng/20 μl, about 2 ng/20 μl to about 2000 ng/20 μl, about 5 ng/20 μl to about 1500 ng/20 μl, about 10 ng/20 μl to about 1500 ng/20 μl, about 25 ng/20 μl to about 1500 ng/20 μl, about 50 ng/20 μl to about 1500 ng/20 μl, about 100 ng/20 μl to about 1500 ng/20 μl, about 250 ng/20 μl to about 1500 ng/20 μl, about 300 ng/20 μl to about 1500 ng/20 μl, about 500 ng/20 μl to about 1500 ng/20 μl, about 500 ng/20 μl to about 1250 ng/20 μl, or about 625 ng/20 μl to about 1250 ng/20 μl. In other embodiments, the compositions may comprise the E. coli ribosomal protein L27, at a concentration of, for example, about 1,000 ng/20 μl to about 50,000 ng/20 μl, about 2,000 ng/20 μl to about 40,000 ng/20 μl, about 5,000 ng/20 μl to about 30,000 ng/20 μl, about 10,000 ng/20 μl to about 25,000 ng/20 μl, about 10,000 ng/20 μl to about 20,000 ng/20 μl, or about 10,000 ng/20 μl. Of course, other concentration ranges for S20, S15, or L27, or other suitable prokaryotic or eukaryotic ribosomal proteins that may be used in the present compositions, may be determined by one of ordinary skill without undue experimentation by carrying out a titration assay as noted above and as described in detail in the Examples below. Further, other ribosomal proteins, or fragments thereof, may be present in compositions of the invention in amounts set out above.

Recombination Proteins

In compositions and methods of the present invention, the exchange of DNA segments is achieved by the use of recombination proteins, including recombinases and associated co-factors and proteins. The one or more recombination proteins for use in compositions of the invention may be any recombination protein, including any prokaryotic or eukaryotic recombination protein, that is suitable for use in recombinational cloning of nucleic acid molecules. Examples of such recombination proteins include, but are not limited to:

A. Cre: A prokaryotic recombination protein from bacteriophage P1 (Abremski and Hoess, J. Biol. Chem. 259:1509-1514 (1984)) catalyzes the exchange (i.e., causes recombination) between 34 base pair DNA sequences called loxP (locus of crossover) sites (See Hoess et al., Nucl. Acids Res. 14:2287 (1986)). Cre is available commercially (Novagen, Inc. 601 Science Drive, Madison, Wis. 53711, Catalog No. 69247-1). Recombination mediated by Cre is freely reversible. From thermodynamic considerations it is not surprising that Cre-mediated integration (recombination between two molecules to form one molecule) is much less efficient than Cre-mediated excision (recombination between two loxP sites in the same molecule to form two daughter molecules). Cre works in simple buffers with either magnesium or spermidine as a cofactor, as is well known in the art. The DNA substrates can be either linear or supercoiled. A number of mutant loxP sites have been described (Hoess et al., Nucl. Acids Res. 14:2287 (1986)). One of these, loxP 511, recombines with another loxP 511 site, but will not recombine with a loxP site.

B. Integrase: A prokaryotic recombination protein from bacteriophage lambda that mediates the integration of the lambda genome into the E. coli chromosome. The bacteriophage λ Int recombinational protein promotes recombination between its substrate att sites as part of the formation or induction of a lysogenic state. Reversibility of the recombination reactions results from two independent pathways for integrative and excisive recombination. Each pathway uses a unique, but overlapping, set of the 15 protein binding sites that comprise att site DNAs. Cooperative and competitive interactions involving four proteins (Int, Xis, IHF and Fis) determine the direction of recombination.

Integrative recombination involves the Int and IF proteins and sites attP (240 base pairs) and attB (25 base pairs). Recombination results in the formation of two new sites: attL and attR. Excisive recombination requires Int, IHF, and Xis, and sites attL and attR to generate attP and attB. Under certain conditions, Fis stimulates excisive recombination. In addition to these normal reactions, it should be appreciated that attP and attB, when placed on the same molecule, can promote excisive recombination to generate two excision products, one with attL and one with attR. Similarly, intermolecular recombination between molecules containing attL and attR, in the presence of Int, IHF and Xis, can result in integrative recombination and the generation of attP and attB. Hence, by flanking DNA segments with appropriate combinations of engineered att sites, in the presence of the appropriate recombination proteins, one can direct excisive or integrative recombination, as reverse reactions of each other.

Each of the att sites contains a 15 base pair core sequence; individual sequence elements of functional significance lie within, outside, and across the boundaries of this common core (Landy, A., Ann. Rev. Biochem. 58:913 (1989)). Efficient recombination between the various att sites requires that the sequence of the central common region be identical between the recombining partners, however, the exact sequence is now found to be modifiable. Consequently, derivatives of the att site with changes within the core are now discovered to recombine as least as efficiently as the native core sequences.

Integrase acts to recombine the attP site on bacteriophage lambda (about 240 base pairs) with the attB site on the E. coli genome (about 25 base pairs) (Weisberg, R. A. and Landy, A. in Lambda II, p. 211 (1983), Cold Spring Harbor Laboratory)), to produce the integrated lambda genome flanked by attL (about 100 base pairs) and attR (about 160 base pairs) sites. In the absence of Xis (see below), this reaction is essentially irreversible. The integration reaction mediated by integrase and IHF works in vitro, with simple buffer containing spermidine. Integrase can be obtained as described by Nash, H. A., Methods of Enzymology 100:210-216 (1983). IHF can be obtained as described by Filutowicz, M., et al., Gene 147:149-150 (1994).

Numerous recombination systems from various organisms (e.g., prokaryotic and eukaryotic systems) can also be used, based on the teaching and guidance provided herein. See, e.g., Hoess et al., Nucleic Acids Research 14(6):2287 (1986); Abremski et al., J. Biol. Chem.261(1):391 (1986); Campbell, J. Bacteriol. 174(23):7495 (1992); Qian et al., J. Biol. Chem. 267(11):7794 (1992); Araki et al., J. Mol. Biol. 225(1):25 (1992)). Many of these belong to the integrase family of recombinases (Argos et al. EMBO J. 5:433-440 (1986)). Perhaps the best studied of these are the Integrase/att system from bacteriophage λ (Landy, A. (1993) Current Opinions in Genetics and Devel. 3:699-707), the Cre/loxP system from bacteriophage P1 (Hoess and Abremski (1990) In Nucleic Acids and Molecular Biology, vol. 4. Eds.: Eckstein and Lilley, Berlin-Heidelberg: Springer-Verlag; pp. 90-109), and the FLP/FRT system from the Saccharomyces cerevisiae 2μ circle plasmid (Broach et al. Cell 29:227-234 (1982)).

Members of the resolvase (Res) family of site-specific recombinases (e.g., γδ, Tn3 resolvase, Hin, Gin, and Cin) are also known, and may be used in accordance with the present invention. Members of this highly related family of recombinases are typically constrained to intramolecular reactions (e.g., inversions and excisions) and can require host-encoded factors. Mutants have been isolated that relieve some of the requirements for host factors (Maeser and Kahnmann (1991) Mol. Gen. Genet. 230:170-176), as well as some of the constraints of intramolecular recombination.

Other site-specific recombinases similar to λ Int and similar to P1 Cre can be substituted for Int and Cre. Such recombinases are known. In many cases the purification of such other recombinases has been described in the art. In cases when they are not known, cell extracts can be used or the enzymes can be partially purified using procedures described for Cre and Int.

While Cre and Int are described in detail for reasons of example, many related recombination systems and proteins exist and their application to the described invention is also provided according to the present invention. The integrase family of site-specific recombinases can be used to provide alternative recombination proteins and recombination sites for the present invention, as site-specific recombination proteins encoded by, for example bacteriophage lambda, phi 80, P22, P2, 186, P4 and P1. This group of recombination proteins, which may be used in the present compositions and methods, exhibits an unexpectedly large diversity of sequences. Despite this diversity, all of these recombinases can be aligned in their C-terminal halves. A 40-residue region near the C terminus is particularly well conserved in all the proteins and is homologous to a region near the C terminus of the yeast 2 mu plasmid FLP recombination protein. Three positions are conserved within many members of this family: histidine, arginine and tyrosine are found at respective alignment positions 396, 399 and 433 within the well-conserved C-terminal region. These residues contribute to the active site of this family of recombinases, and suggest that tyrosine-433 forms a transient covalent linkage to DNA during strand cleavage and rejoining. See, e.g., Argos, P. et al., EMBO J. 5:433-40 (1986).

The recombinases of some transposons, such as those of conjugative transposons (e.g., Tn916) (Scott and Churchward, Ann. Rev. Microbiol. 49:367 (1995); Taylor and Churchward, J. Bacteriol. 179:1837 (1997)), may also be used in the compositions and methods of the invention. These transposon recombinases belong to the integrase family of recombinases and in some cases show strong preferences for specific integration sites (Ike et al., J. Bacteriol. 174:1801 (1992); Trieu-Cuot et al., Mol. Microbiol. 8:179 (1993)).

Alternatively, IS231 and other Bacillus thuringiensis transposable elements could be used in accordance with the present invention as recombination proteins and recombination sites. Bacillus thuringiensis is an entomopathogenic bacterium whose toxicity is due to the presence in the sporangia of delta-endotoxin crystals active against agricultural pests and vectors of human and animal diseases. Most of the genes coding for these toxin proteins are plasmid-borne and are generally structurally associated with insertion sequences (IS231, IS232, IS240, ISBT1 and ISBT2) and transposons (Tn4430 and Tn5401). Several of these mobile elements have been shown to be active and participate in the crystal gene mobility, thereby contributing to the variation of bacterial toxicity.

Structural analysis of the iso-IS231 elements indicates that they are related to IS1151 from Clostridium perfringens and distantly related to IS4 and IS186 from Escherichia coli. Like the other IS4 family members, they contain a conserved transposase-integrase motif found in other IS families and retroviruses. Moreover, functional data gathered from IS231A in Escherichia coli indicate a non-replicative mode of transposition, with a preference for specific targets. Similar results were also obtained in Bacillus subtilis and B. thuringiensis. See, e.g., Mahillon, J. et al., Genetica 93:13-26 (1994); Campbell, J. Bacteriol. 7495-7499 (1992).

An unrelated family of recombinases, the transposases, have also been used to transfer genetic information between replicons, and may therefore be used as recombination proteins in accordance with the invention. Transposons are structurally variable, being described as simple or compound, but typically encode the recombinase gene flanked by DNA sequences organized in inverted orientations. Integration of transposons can be random or highly specific. Representatives such as Tn7, which are highly site-specific, have been applied to the efficient movement of DNA segments between replicons (Lucklow et al., J. Virol 67:4566-4579 (1993)).

A related element, the integron, are also translocatable-promoting movement of drug resistance cassettes from one replicon to another. Often these elements are defective transposon derivatives. Transposon Tn21 contains a class I integron called In2. The integrase (IntI1) from In2 is common to all integrons in this class and mediates recombination between two 59-bp elements or between a 59-bp element and an attI site that can lead to insertion into a recipient integron. The integrase also catalyzes excisive recombination. (Hall, Ciba Found. Symp. 207:192 (1997); Francia et al., J. Bacteriol. 179:4419 (1997)).

Group II introns are mobile genetic elements encoding a catalytic RNA and protein. The protein component possesses reverse transcriptase, maturase and an endonuclease activity, while the RNA possesses endonuclease activity and determines the sequence of the target site into which the intron integrates. By modifying portions of the RNA sequence, the integration sites into which the element integrates can be defined. Foreign DNA sequences can be incorporated between the ends of the intron, allowing targeting to specific sites. This process, termed “retrohoming,” occurs via a DNA:RNA intermediate, which is copied into cDNA and ultimately into double stranded DNA (Matsuura et al., Genes and Dev. 11:2910-2924 (1997); Guo et al., EMBO J. 16:6835-6848 (1997)). Numerous intron-encoded homing endonucleases have been identified (Belfort and Roberts, Nucl. Acids. Res. 25:3379 (1997)). Such systems can be easily adopted for application to the subcloning methods described herein.

In addition, other suitable recombination proteins are described in detail in U.S. Pat. No. 5,851,808; U.S. application Ser. No. 08/486,139, filed Jun. 7, 1995; U.S. application Ser. No. 08/663,002, filed Jun. 7, 1996 (now U.S. Pat. No. 5,888,732); U.S. application Ser. No. 09/005,476, filed Jan. 12, 1998; U.S. Application No. 60/065,930, filed Oct. 24, 1997; U.S. application Ser. No. 09/177,387, filed Oct. 23, 1998; U.S. Application No. 60/122,389, filed Mar. 2, 1999; U.S. Application No. 60/122,392, filed Mar. 22, 1999; U.S. Application No. 60/126,049, filed Mar. 23, 1999; U.S. application Ser. No. 09/438,358, filed Nov. 12, 1999; U.S. Application No. 60/136,744, filed May 28, 1999; U.S. application Ser. No. 09/517,466, filed Mar. 2, 2000; U.S. application Ser. No. 09/732,914, filed Dec. 11, 2000; and PCT Publication No. WO 00/52027, the disclosures of all of which are incorporated herein by reference in their entireties.

In certain compositions of the invention, the recombination protein may be selected from the group consisting of Int, Cre, Res, Xis, FLP, IHF and HU, and may be a site-specific recombination protein.

The amount of recombination protein which is optimal for use in the compositions and methods of the present invention for enhancing the efficiency of recombination reactions can be determined using known assays. Specifically, a titration assay may be used to determine the appropriate amount of a purified recombination protein, or the appropriate amount of an extract. Such assays are described in the Examples below.

In certain exemplary compositions of the invention, the compositions comprise an effective amount of λ Int, for example at a concentration range of about 1 ng/20 μl to about 500 ng/20 μl, about 2 ng/20 μl to about 250 ng/20 μl, about 5 ng/20 μl to about 200 ng/20 μl, about 10 ng/20 μl to about 200 ng/20 μl, about 25 ng/20 μl to about 200 ng/20 μl, about 50 ng/20 μl to about 200 ng/20 μl, or about 100 ng/20 μl to about 200 ng/20 μl.

In addition, compositions of the invention may comprise one or more additional recombination proteins, such as a composition of the invention may comprise λ Int at the above-indicated concentration ranges, and HU protein and/or IHF protein at concentrations of about 1 ng/20 μl, about 5 ng/20 μl, about 10 ng/20 μl, about 20 ng/20 μl, about 30 ng/20 μl, about 40 ng/20 μl, about 50 ng/20 μl, about 60 ng/20 μl, about 70 ng/20 μl, about 80 ng/20 μl, about 90 ng/20 μl, about 100 ng/20 μl, about 110 ng/20 μl, about 120 ng/20 μl, about 130 ng/20 μl, or about 140 ng/20 μl or concentration ranges of about 1 ng/20 μl to about 50 ng/20 μl, about 2 ng/20 μl to about 25 ng/20 μl, about 5 ng/20 μl to about 20 ng/20 μl, about 5 ng/20 μl to about 15 ng/20 μl, about 5 ng/20 μl to about 10 ng/20 μl, about 5 ng/20 μl to about 120 ng/20 μl, about 5 ng/20 μl to about 100 ng/20 μl, about 5 ng/20 μl to about 80 ng/20 μl, about 5 ng/20 μl to about 70 ng/20 μl, about 5 ng/20 μl to about 60 ng/20 μl, about 10 ng/20 μl to about 20 ng/20 μl, about 10 ng/20 μl to about 40 ng/20 μl, about 10 ng/20 μl to about 50 ng/20 μl, about 10 ng/20 μl to about 60 ng/20 μl, about 10 ng/20 μl to about 70 ng/20 μl, about 10 ng/20 μl to about 80 ng/20 μl, about 10 ng/20 μl to about 90 ng/20 μl, or about 10 ng/20 μl to about 100 ng/20 μl. Of course, other concentration ranges for λ Int or other recombination proteins that may be used in the present compositions may be determined by one of ordinary skill, without undue experimentation, by carrying out a titration assay as noted above and as described in detail in the Examples below.

DNA Molecules, Vectors and Host Cells

The above-described compositions of the invention are suitable for use in recombination cloning methods that are provided by the present invention. Recombinational cloning using nucleic acid molecules comprising engineered recombination sites, and the materials and methods by which this technique may be accomplished, have been described in detail in U.S. application Ser. No. 08/486,139, filed Jun. 7, 1995; U.S. application Ser. No. 08/663,002, filed Jun. 7, 1996 (now U.S. Pat. No. 5,888,732); U.S. application Ser. No. 09/005,476, filed Jan. 12, 1998; U.S. Application No. 60/065,930, filed Oct. 24, 1997; U.S. application Ser. No. 09/177,387, filed Oct. 23, 1998; U.S. Application No. 60/122,389, filed Mar. 2, 1999; U.S. Application No. 60/122,392, filed Mar. 22, 1999; U.S. Application No. 60/126,049, filed Mar. 23, 1999; U.S. application Ser. No. 09/438,358, filed Nov. 12, 1999; U.S. Application No. 60/136,744, filed May 28, 1999; U.S. application Ser. No. 09/517,466, filed Mar. 2, 2000; and PCT Publication No. WO 00/52027, the disclosures of all of which are incorporated herein by reference in their entireties.

According to the invention, the Insert Donor molecules for use in the compositions and methods of the invention may be derived from genomic DNA or cDNA, or may be produced by chemical synthesis methods. In a related aspect, the Insert Donor molecules may comprise one or more vectors.

The Vector Donor molecules, as well as other nucleic acid molecules, for use in the compositions and methods of the invention may optionally comprise at least one Selectable marker, which allows for the selection of host cells comprising desired molecules, such as Cointegrate or intermediate molecules and Product molecules comprising the Selectable markers contributed by the Vector Donor molecules during the recombination reaction. According to this aspect of the invention, the Selectable Marker may be an antibiotic resistance gene, a tRNA gene, an auxotrophic marker, a toxic gene, a phenotypic marker, an antisense oligonucleotide, a restriction endonuclease, a restriction endonuclease cleavage site, an enzyme cleavage site, a protein binding site, and a sequence complementary to a PCR primer sequence. In a related aspect, the Vector Donor molecules, as well as other nucleic acid molecules, may comprise one or more eukaryotic vectors or one or more prokaryotic vectors. Eukaryotic vectors suitable for use in this aspect of the invention may comprise, for example, vectors which propagate and/or replicate in yeast cells, plant cells, fish cells, eukaryotic cells, mammalian cells, and/or insect cells, while suitable prokaryotic vectors may comprise, for example, vectors which propagate and/or replicate in bacteria of the genera Escherichia (most particularly E. coli), Salmonella, Bacillus, Streptomyces or Pseudomonas.

The compositions and methods described herein are suitable for use in recombination cloning according to the present invention. However, wild-type recombination sites that are contained in the Insert Donor and/or Vector Donor DNA molecules, as well as other nucleic acid molecules, may contain sequences that reduce the efficiency or specificity of recombination reactions or the function of the Product molecules as applied in methods of the present invention. For example, multiple stop codons in attB, attR, attP, attL and loxP recombination sites occur in multiple reading frames on both strands, so translation efficiencies are reduced, e.g., where the coding sequence must cross the recombination sites, (only one reading frame is available on each strand of loxP and attB sites) or impossible (in attP, attR or attL).

Accordingly, nucleic acid molecules comprising one or more engineered recombination sites may be used in the methods of the present invention, to overcome these problems. For example, att sites can be engineered to have one or multiple mutations to enhance specificity or efficiency of the recombination reaction and the properties of Product DNAs (e.g., att1, att2, and att3 sites); to decrease reverse reaction (e.g., removing P1 and H1 from attR). The testing of these mutants determines which mutants yield sufficient recombinational activity to be suitable for recombination subcloning according to the present invention. Hence, in addition to the one or more Fis proteins or Fis protein fragments (and optionally one or more recombination proteins and/or one or more ribosomal proteins or ribosomal protein fragments described herein), compositions of the invention may further comprise one or more nucleic acid molecules including, but not limited to, one or more Insert Donor molecules, one or more Vector Donor molecules, one or more Cointegrate molecules, one or more Product molecules and one or more Byproduct molecules, any or all of which may contain engineered or mutant recombination sites.

Mutations can be introduced into recombination sites for enhancing site specific recombination. The production of DNA molecules comprising one or more mutated engineered recombination sites, which molecules may be used as Insert Donor or Vector Donor molecules in the recombinational cloning methods of the present invention, is described in detail in U.S. application Ser. No. 08/486,139, filed Jun. 7, 1995; U.S. application Ser. No. 08/663,002, filed Jun. 7, 1996 (now U.S. Pat. No. 5,888,732); U.S. application Ser. No. 09/005,476, filed Jan. 12, 1998; U.S. Application No. 60/065,930, filed Oct. 24, 1997; U.S. application Ser. No. 09/177,387, filed Oct. 23, 1998; U.S. Application No. 60/122,389, filed Mar. 2, 1999; U.S. Application No. 60/122,392, filed Mar. 22, 1999; U.S. Application No. 60/126,049, filed Mar. 23, 1999; U.S. application Ser. No. 09/438,358, filed Nov. 12, 1999; U.S. Application No. 60/136,744, filed May 28, 1999; U.S. application Ser. No. 09/517,466, filed Mar. 2, 2000; and PCT Publication No. WO 00/52027, the disclosures of all of which are incorporated herein by reference in their entireties. In particular embodiments, compositions and methods of the invention either comprise or use nucleic acid molecules comprising at least one nucleic acid segment having at least two engineered recombination sites flanking a Selectable marker and/or a desired DNA segment, wherein at least one of the recombination sites comprises a core region having at least one engineered mutation that enhances recombination in vitro in the formation of a Cointegrate DNA or a Product DNA.

In accordance with the invention, any vector may be used to construct the Vector Donors used in the methods of the invention. In particular, vectors known in the art and those commercially available (and variants or derivatives thereof) may in accordance with the invention be engineered to include one or more recombination sites for use in the methods of the invention. Such vectors may be obtained from, for example, Vector Laboratories Inc., Invitrogen, Promega, Novagen, NEB, Clontech, Boehringer Mannheim, Pharmacia, EpiCenter, OriGenes Technologies Inc., Stratagene, Perkin Elmer, Pharmingen, and Research Genetics. Such vectors may then for example be used for cloning or subcloning nucleic acid molecules of interest. General classes of vectors of particular interest include prokaryotic and/or eukaryotic cloning vectors, expression vectors, fusion vectors, two-hybrid or reverse two-hybrid vectors, shuttle vectors for use in different hosts, mutagenesis vectors, transcription vectors, vectors for receiving large inserts and the like. Exemplary vectors (and mutants, derivatives, or variants thereof) that may be used to construct the Vector Donors used in the methods of the invention are described in detail in U.S. application Ser. No. 08/486,139, filed Jun. 7, 1995; U.S. application Ser. No. 08/663,002, filed Jun. 7, 1996 (now U.S. Pat. No. 5,888,732); U.S. application Ser. No. 09/005,476, filed Jan. 12, 1998; U.S. Application No. 60/065,930, filed Oct. 24, 1997; U.S. application Ser. No. 09/177,387, filed Oct. 23, 1998; U.S. Application No. 60/122,389, filed Mar. 2, 1999; U.S. Application No. 60/122,392, filed March22, 1999; U.S. Application No. 60/126,049, filed Mar. 23, 1999; U.S. application Ser. No. 09/438,358, filed Nov. 12, 1999; U.S. Application No. 60/136,744, filed May 28, 1999; U.S. application Ser. No. 09/517,466, filed Mar. 2, 2000; and PCT Publication No. WO 00/52027, the disclosures of all of which are incorporated herein by reference in their entireties.

The invention also relates generally to DNA molecules produced by the methods of the invention, particularly to such DNA molecules which are isolated DNA molecules. Methods for the isolation of DNA molecules produced by the methods of the invention will be familiar to one of ordinary skill in the art, and are described generally in U.S. application Ser. No. 08/486,139, filed Jun. 7, 1995; U.S. application Ser. No. 08/663,002, filed Jun. 7, 1996 (now U.S. Pat. No. 5,888,732); U.S. application Ser. No. 09/005,476, filed Jan. 12, 1998; U.S. Application No. 60/065,930, filed Oct. 24, 1997; U.S. application Ser. No. 09/177,387, filed Oct. 23, 1998; U.S. Application No. 60/122,389, filed Mar. 2, 1999; U.S. Application No. 60/122,392, filed Mar. 22, 1999; U.S. Application No. 60/126,049, filed Mar. 23, 1999; U.S. application Ser. No. 09/438,358, filed Nov. 12, 1999; U.S. Application No. 60/136,744, filed May 28, 1999; U.S. application Ser. No. 09/517,466, filed Mar. 2, 2000; and PCT Publication No. WO 00/52027, the disclosures of all of which are incorporated herein by reference in their entireties. In addition, the isolated DNA molecules of the invention may be inserted into standard nucleotide vectors suitable for transfection or transformation of a variety of prokaryotic (bacterial) or eukaryotic (yeast, plant or animal including human and other mammalian) host cells. Vectors suitable for these purposes, and methods for insertion of DNA fragments therein, will be well-known to one of ordinary skill in the art. Thus, the present invention also relates to vectors comprising such DNA molecules, and to host cells comprising such DNA molecules and/or vectors.

Representative host cells that may be used with the invention include, but are not limited to, bacterial cells, yeast cells, plant cells and animal cells. Bacterial host cells suitable for use with the invention include Escherichia spp. cells (particularly E. coli cells and most particularly E. coli strains DH10B, Stb12, DH5α, DB3, DB3.1 (e.g., E. coli LIBRARY EFFICIENCY® DB3.1™ Competent Cells; Invitrogen Corp., Life Technologies Division (Rockville, Md.)), DB4 and DB5; see U.S. application Ser. No. 09/518,188, filed on Mar. 2, 2000, the disclosure of which is incorporated by reference herein in its entirety), Bacillus spp. cells (particularly B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells). Animal host cells suitable for use with the invention include insect cells (most particularly Drosophila melanogaster cells, Spodoptera frugiperda Sf9 and Sf21 cells and Trichoplusa High-Five cells), nematode cells (particularly C. elegans cells), avian cells, amphibian cells (particularly Xenopus laevis cells), reptilian cells, and mammalian cells (most particularly CHO, COS, VERO, BHK and human cells). Yeast host cells suitable for use with the invention include Saccharomyces cerevisiae cells and Pichia pastoris cells. These and other suitable host cells are available commercially, for example from Invitrogen Corp., Life Technologies Division (Rockville, Md.); the American Type Culture Collection (Manassas, Va.); and the Agricultural Research Culture Collection (NRRL; Peoria, Ill.).

Methods for introducing the nucleic acid molecules and/or vectors of the invention into the host cells described herein, to produce host cells comprising one or more of the nucleic acid molecules and/or vectors of the invention, will be familiar to those of ordinary skill in the art. For instance, the nucleic acid molecules and/or vectors of the invention may be introduced into host cells using well known techniques of infection, transduction, transfection, and transformation. The nucleic acid molecules and/or vectors of the invention may be introduced alone or in conjunction with other the nucleic acid molecules and/or vectors. Alternatively, the nucleic acid molecules and/or vectors of the invention may be introduced into host cells as a precipitate, such as a calcium phosphate precipitate, or in a complex with a lipid. Electroporation also may be used to introduce the nucleic acid molecules and/or vectors of the invention into a host. Likewise, such molecules may be introduced into chemically competent cells such as E. coli. If the vector is a virus, it may be packaged in vitro or introduced into a packaging cell and the packaged virus may be transduced into cells. Hence, a wide variety of techniques suitable for introducing the nucleic acid molecules and/or vectors of the invention into cells in accordance with this aspect of the invention are well known and routine to those of skill in the art. Such techniques are reviewed at length, for example, in Sambrook, J., et al., Molecular Cloning, a Laboratory Manual, 2nd Ed., Cold Spring Harbor, N.Y.: Cold Spring Harbor Laboratory Press, pp. 16.30-16.55 (1989), Watson, J. D., et al., Recombinant DNA, 2nd Ed., New York: W. H. Freeman and Co., pp. 213-234 (1992), and Winnacker, E., From Genes to Clones, New York: VCH Publishers (1987), which are illustrative of the many laboratory manuals that detail these techniques and which are incorporated by reference herein in their entireties for their relevant disclosures.

Kits

The invention also relates to kits for use in recombinational cloning of nucleic acid molecules. Kits according to the present invention may comprise a carrying means being compartmentalized to receive in close confinement therein one or more containers such as vials, tubes, bottles, ampules and the like. Each of such containers may comprise components or a mixture of components needed to perform recombinational cloning of nucleic acid molecules, particularly according to the methods of the present invention.

In one such aspect, the kits of the invention may comprise at least one Fis protein or fragment thereof. Additionally, such kits may comprise at least one recombination protein. Fis proteins and recombination proteins suitable for use in the kits of the invention include, but are not necessarily limited to, the proteins described in detail herein. Of course, it is also possible to combine one or more of these components into a single container, such that the kit will contain one or more containers wherein a first container contains at least one Fis protein and at least one recombination protein, or wherein a first container contains one or more of the above-described compositions or components of the invention. Additional kits of the invention may comprise one or more additional containers containing additional components which may be useful in carrying out recombinational cloning of nucleic acid molecules, including, for example, one or more polymerases (such as one or more thermostable DNA polymerases like Taq, Tne, Tma, and the like), one or more ribosomal proteins (or fragments thereof), one or more polypeptides having reverse transcriptase activity (such as RSV or ASLV reverse transcriptases, particularly those that are substantially reduced in RNase H activity), one or more restriction endonucleases, one or more buffers, one or more detergents, instructions for use of kit components, and the like.

Applications

There are a number of applications for the compositions, methods and kits of the present invention. These uses include, but are not limited to, changing vectors, operably linking genes to regulatory genetic sequences (e.g., promoters, enhancers, and the like), constructing genes for fusion proteins, changing copy number, changing replicons, cloning into phages, and cloning, e.g., PCR products (with an attB site at one end and a loxP site at the other end), genomic DNAs, and cDNAs. Such applications are described in detail, for example, in U.S. application Ser. No. 08/486,139, filed Jun. 7, 1995; U.S. application Ser. No. 08/663,002, filed Jun. 7, 1996 (now U.S. Pat. No. 5,888,732); U.S. application Ser. No. 09/005,476, filed Jan. 12, 1998; U.S. Application No. 60/065,930, filed Oct. 24, 1997; U.S. application Ser. No. 09/177,387, filed Oct. 23, 1998; U.S. Application No. 60/122,389, filed Mar. 2, 1999; U.S. Application No. 60/122,392, filed Mar. 22, 1999; U.S. Application No. 60/126,049, filed Mar. 23, 1999; U.S. application Ser. No. 09/438,358, filed Nov. 12, 1999; U.S. Application No. 60/136,744, filed May 28, 1999; U.S. application Ser. No. 09/517,466, filed Mar. 2, 2000; and PCT Publication No. WO 00/52027, the disclosures of all of which are incorporated herein by reference in their entireties.

It will be understood by one of ordinary skill in the relevant arts that other suitable modifications and adaptations to the methods and applications described herein are readily apparent and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The present recombinational cloning methods accomplish the exchange of nucleic acid segments to render something useful to the user, such as a change of cloning vectors. In most instances, these segments must be flanked on both sides by recombination signals that are in the proper orientation with respect to one another. In the examples below the two parental nucleic acid molecules (e.g., plasmids) are called the Insert Donor and the Vector Donor. The Insert Donor contains a segment that will become joined to a new vector contributed by the Vector Donor. The recombination intermediate(s) that contain(s) both starting molecules is called the Cointegrate(s). The second recombination event produces two daughter molecules, called the Product (the desired new clone) and the Byproduct.

Buffers

Various known buffers can be used in the reactions of the present invention. For restriction enzymes, it is advisable to use the buffers recommended by the manufacturer. Alternative buffers can be readily found in the literature or can be devised by those of ordinary skill in the art. One exemplary buffer for lambda integrase is comprised of 50 mM Tris-HCl (pH 7.5-7.8), 70 mM KCl, 5 mM spermidine, 0.5 mM EDTA, and 0.25 mg/ml bovine serum albumin, and optionally, 10% glycerol. Another buffer for lambda integrase is 50 mM Tris-HCl (pH 7.5), 50 mM NaCl, 4 mM spermidine, 1.0 mM EDTA, and 15% glycerol. Suitable buffers for other site-specific recombinases which are similar to lambda Int are either known in the art or can be determined empirically by the ordinarily skilled artisan, particularly in light of the above-described buffers.

### Example 1

### Stimulation of Integrase by E. coli Ribosomal Proteins

Materials and Methods

DNAs for Recombination Assays.

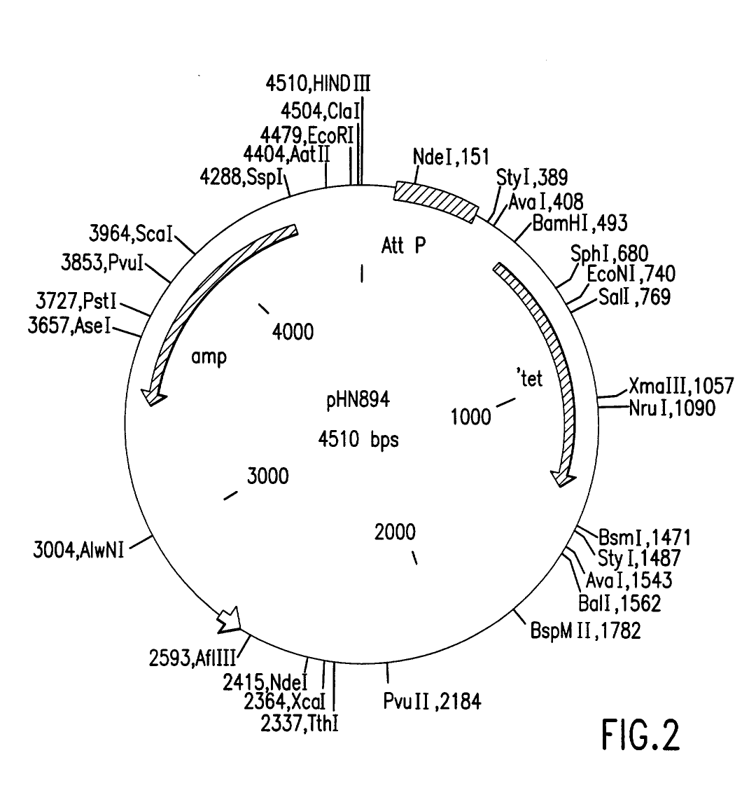

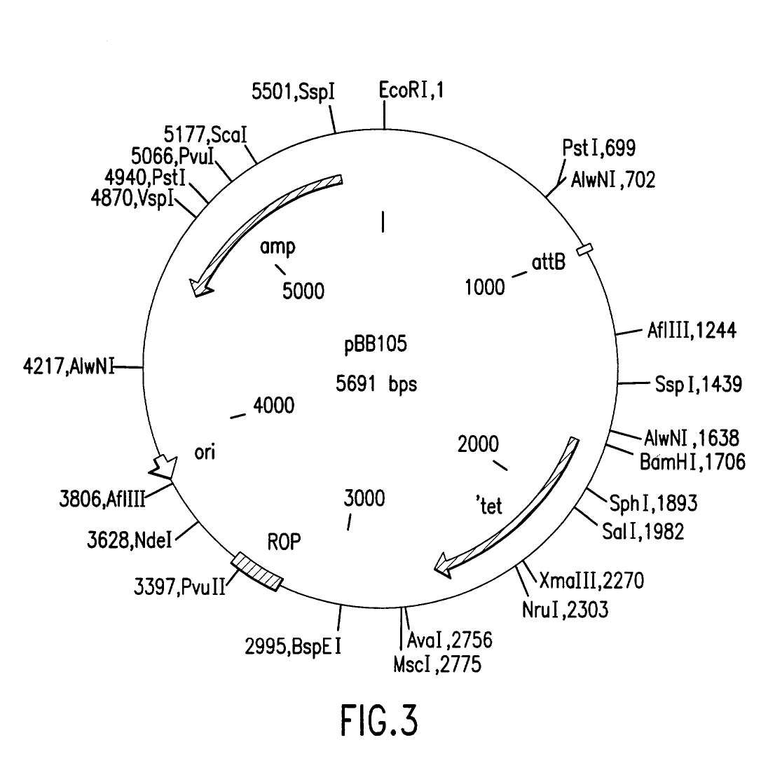

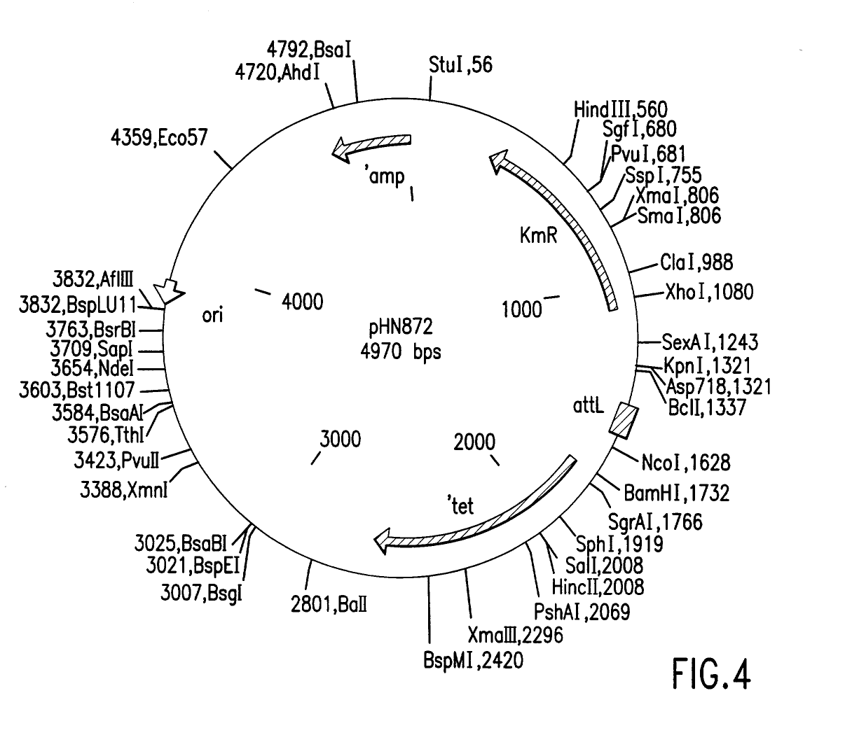

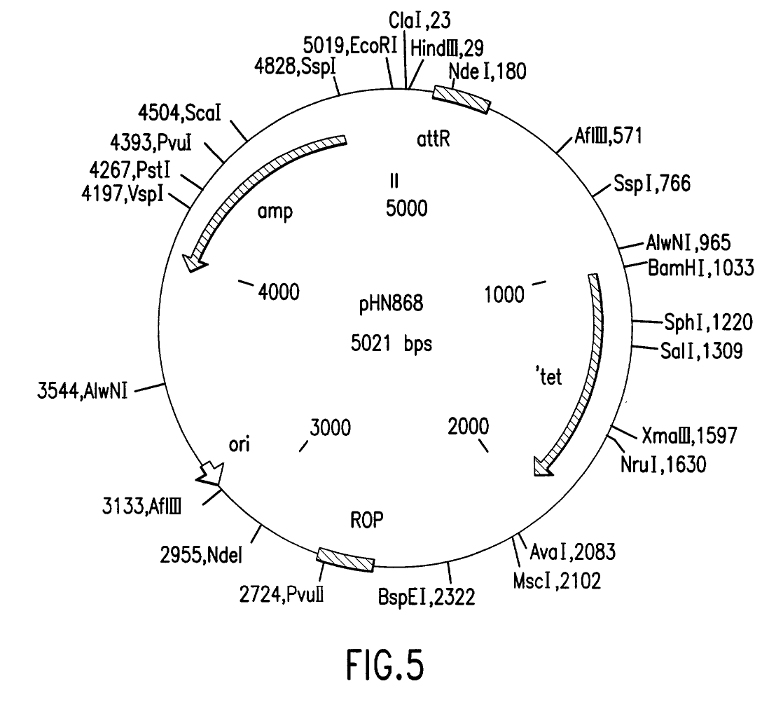

Plasmid pHN894 (FIG. 2), bearing an attP site, and plasmid pBB105 (FIG. 3), bearing an attB site, are described (Kitts, P. A. and Nash, H. A. J. Mol. Biol. 204: 95-107 (1988); Nash, H. A. Methods Enz. 100: 210-216 (1983)). pBB105 was cut with EcoRI before use. Plasmid pHN872 (FIG. 4), bearing an attL site, and plasmid pHN868 (FIG. 5), bearing an attR site, are described (Kitts, P. A. and Nash, H. A. J. Mol. Biol. 204: 95-107 (1988)). pHN872 was cut with SalI before use. These plasmids were propagated in E. coli strain DH10B. To grow cells for preparation of plasmid DNA, the growth medium contained in one liter: 12 g of tryptone, 24 g of yeast extract, 2.3 g of KH₂PO₄, 12.5 g of K₂HPO₄, 0.01% (v/v) PPG antifoam, and appropriate antibiotic. Cells from a glycerol seed were placed in 25 ml of medium containing 100 μg/ml ampicillin (pBB105, pHN894, pHN868) or 100 μg/ml kanamycin (pHN872) and grown overnight at 37° C. Fifteen ml of overnight culture was added to 1.5 L medium containing 10 μg/ml appropriate antibiotic and cells were grown to a A₆₀₀ of ˜2.0. Chloramphenicol was then added to a final concentration of 170 μg/ml and growth was continued for 16 hr at 37° C. Cells were harvested by centrifugation and stored at −70° C. Plasmid DNAs were purified as follows. Frozen cells were thawed on ice and suspended in 7 ml/g cells of 25 mM Tris-HCl (pH 8.0), 10 mM EDTA, and 50 mM glucose (TEG)+100 μg/ml of RNaseA+1 mg/ml lysozyme. A solution of 1% (w/v) SDS-0.125 N NaOH at 14 ml/g cells was then added to lyse cells. After 10 minutes on ice, 7.5 M ammonium acetate at 10.5 ml/g cells was added. After 10 minutes on ice, the mixture was centrifuged at 28,000× g for 10 minutes and the supernatant was collected. DNA was precipitated by addition of 0.6 volumes of cold isopropanol, and DNA was pelleted by centrifugation at 28,000× g for 10 minutes. The DNA pellet was dissolved in 10 mM Tris-HCl(pH 7.5)−1 mM EDTA(T₁₀E₁)+RNase A (100 μg/ml)+RNaseT1(1,200 U/ml). After phenol extraction and ethanol precipitation of the DNA, it was dissolved in T₁₀E₁. The DNA was dialyzed against 100 volumes of 10 mM Tris-HCl (pH 7.5), 1 mM EDTA, and 450 mM NaCl (T₁₀E₁N₄₅₀) overnight. The dialyzed DNA was applied to a NACS-37 column (LTI) equilibrated in T₁₀E₁N₄₅₀. The column was washed with 10 column volumes of T₁₀E₁N₄₅₀ and eluted with a 15-column volume linear gradient from 0.45 M to 0.65 M NaCl in T₁₀E₁. Fractions were analyzed by agarose gel electrophoresis and those containing supercoiled DNA were pooled. The pooled DNA was dialyzed against T₁₀E₁ and stored at −20° C.

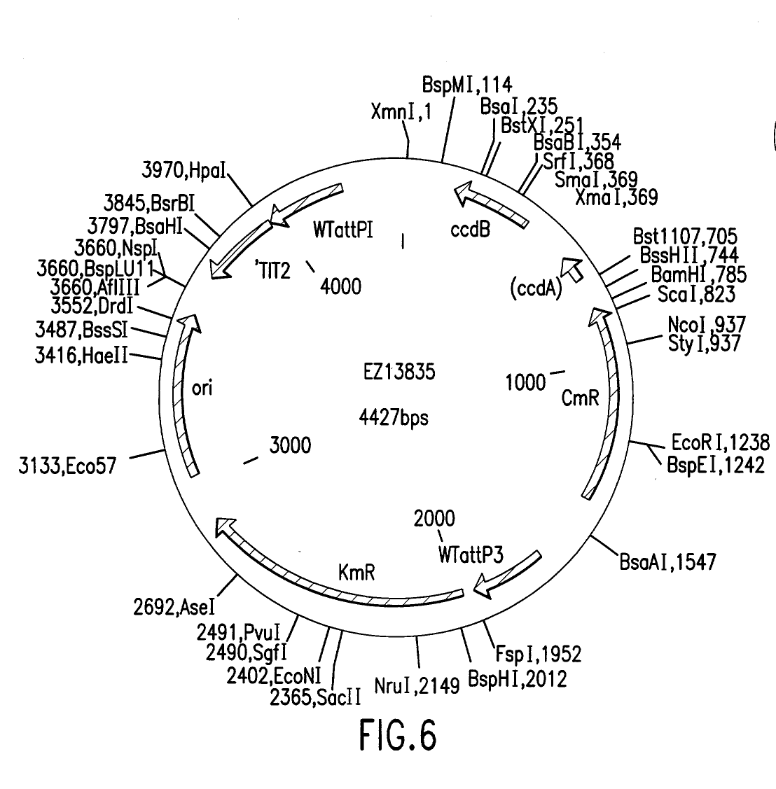

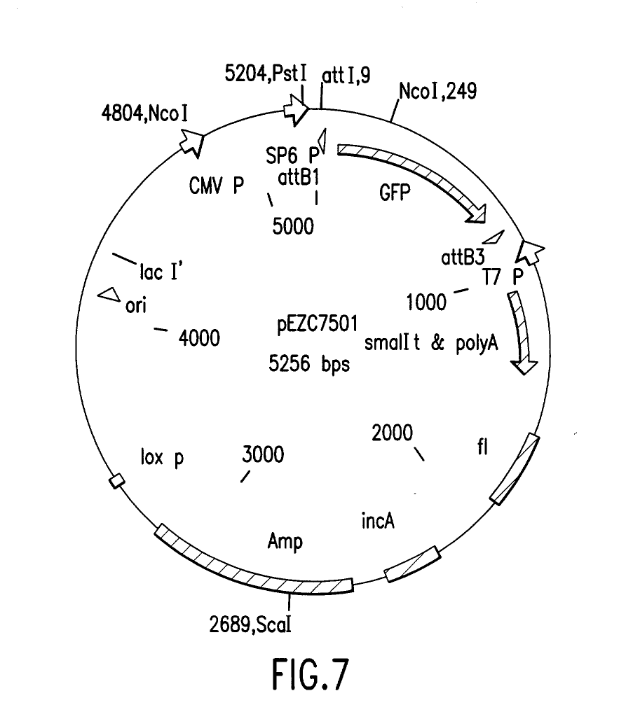

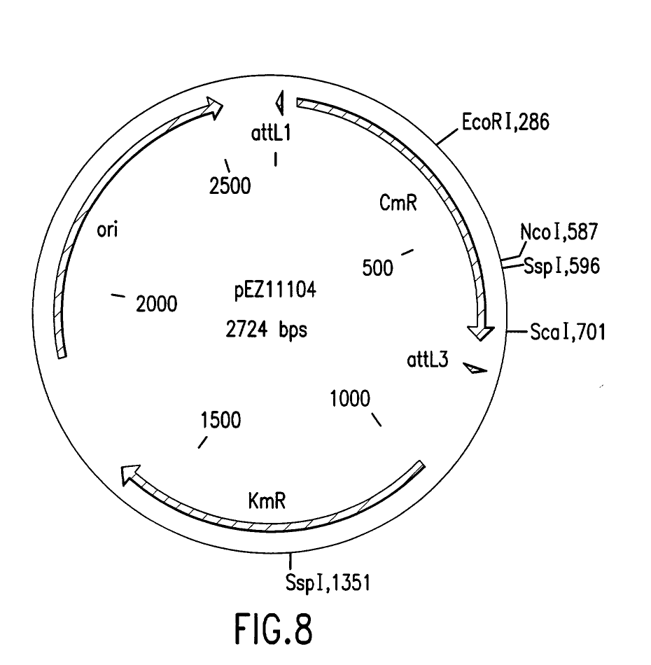

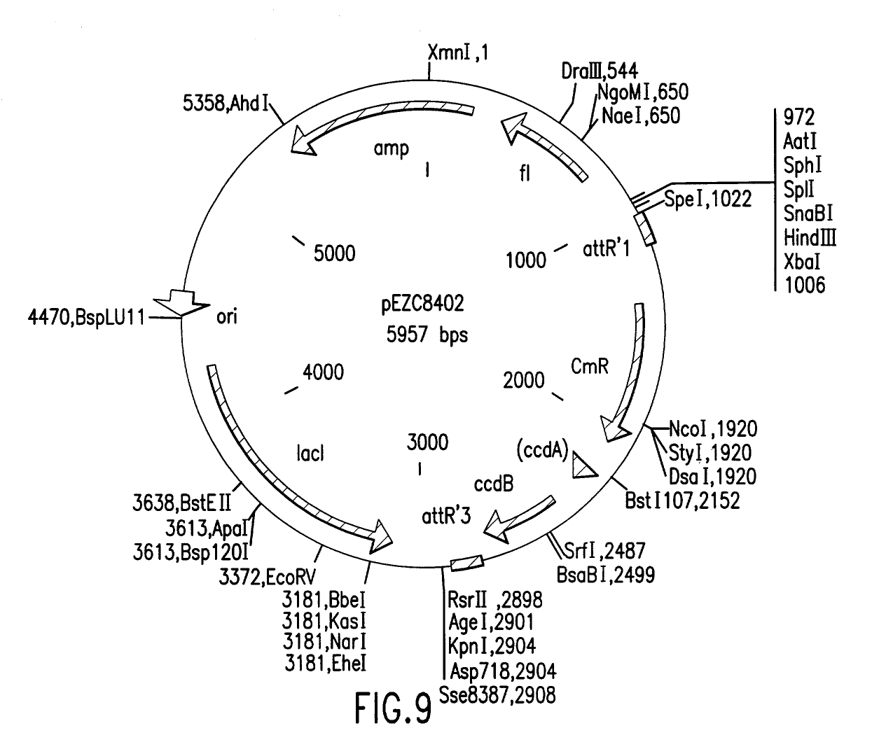

Plasmid pEZ13835 (FIG. 6; attP), pEZC7501 (FIG. 7; attB), pEZ11104 (FIG. 8; attR), and pEZC8402 (FIG. 9; attL) were as shown. pEZC7501 was cut with Seal and pEZC8402 with NcoI before use. pEZ13835 and pEZC8402 were propagated in E. coli DB2 and the other two in E. coli DH5α. Cells from a glycerol seed were placed in 25 ml of Circlegrow (BIO 101) plus 100 μg/ml ampicillin (pEZC7501 and pEZC8402) or plus 100 μg/ml kanamycin (pEZ13835 and pEZ11104) and grown overnight at 37° C. Cells were harvested by centrifugation and stored at −70° C.

Plasmid DNAs were purified using Qiagen Midi products and protocols.

SDS PAGE

Tris-Tricine SDS PAGE 16% precast mini gels (Novex) were used to analyze protein samples. The samples were prepared by mixing with an equal volume of 0.9 M Tris-HCl (pH 8.45), 24% (v/v) glycerol, 8% (w/v) SDS, 0.015% (w/v) Coomassie BlueG, 0.005% (w/v) Phenol Red, and 0.05 M dithiothreitol and boiling for 3 to 5 min. Gels were run at 125 volts in 0.1 M Tris-Tricine (pH 8.3)-0.1% (w/v) SDS for 90 min. Gels were stained in 50% (v/v) methanol, 10% (v/v) acetic acid, and 1 mg/ml Coomassie Blue R-250 solution followed by destaining in 20% (v/v) methanol, 10% (v/v) acetic acid solution.

Determination of Protein Concentration

S20, Int, and Xis bind Bradford reagent dye poorly, so that the Bradford procedure was not used to determine protein concentration. Rather, for Int and Xis, protein concentration was estimated by comparison to Coomassie Blue-stained band intensities of a know amount of BenchMark protein standard of a similar size run along with Int or Xis on an SDS gel. For S20, protein concentration was established using an extinction coefficient at 278 nm of 0.140×10⁴M⁻¹ cm⁻¹ (Eur. J. Biochem. 126: 299-309 (1982)).

PCR.

PCR reaction mixtures (50 μl) contained 22 mM Tris-HCl (pH 8.4), 55 mM KCl, 1.65 mM MgCl₂, 200 μM each of dATP, dCTP, dTTP, and dGTP, 1 μM of each primer, 300 ng of DNA template, and 1.1 units of Taq DNA polymerase. Initial template denaturation was at 95° C. for 5 minutes.

Purification of IHF.

The strain used for overproduction of IHF is described (Nash, H. A. et. al. J. Bacteriol. 169: 4121-4127 (1987)). IHF was purified as described (Rice, P. A. et. al. Cell 87: 1295-1306 (1996)).

Purification of Native Int

Native Int was purified from E. coli strain HN695 (Lange-Gustafson, B. J. and Nash, H. A. J. Biol. Chem. 259:12724-12732 (1984)) by a modification of published procedures (Nash, H. A. Methods Enz. 100:210-216 (1983)).

Growth of Cells

Cells from a glycerol stock of strain HN695 were inoculated into 50 ml of LB broth containing 25 μg/ml ampicillin in a 250-ml flask. The culture was grown at 31° C. in an air shaker to an A₆₅₀ of 0.6 to 1.4. This seed culture was used to inoculate six 2.8-L flasks containing 500 ml of growth medium each and cells were grown as just stated. These cultures were used to inoculate 360 L of growth medium in a 500-L fermentor. Cells were grown at 31° C. with aeration (190 rpm) and agitation (200 rpm) to an A₆₅₀ of 0.65, and were harvested in a chilled centrifuge. Cell paste (˜400 g) was brought to 600 ml by addition of ice-cold 50 mM Tris-HCl (pH 7.5) containing 10% (w/v) sucrose and homogenized in a Waring blender at low speed. The slurry was divided into 40-ml aliquots, frozen in dry ice, and stored at −70° C.

Preparation of Extract.

Three tubes of frozen cells (60 g) were thawed at room temperature and placed on ice. To each tube, 2 ml of a 10 mg/ml solution of lysozyme in 250 mM Tris-HCl (pH 7.5) was added, and the tubes were mixed thoroughly. After 35 min on ice, the mixture was centrifuged at 32,600× g for 45 min. The supernatant was retained (57 ml).

Differential Salt Precipitation

The supernatant was diluted with 50 mM Tris-HCl (pH 7.5) to 100 ml and centrifuged at 4° C. and 41,000 rpm (170,000× g) for 200 min in a precooled Sorval T865 rotor. The supernatant was decanted, frozen, and stored at −70° C. The pellet was stored at −70° C. Thawed pellet was resuspended with the aid of a Teflon pestle in Buffer X (50 mM Tris-HCl (pH 7.5), 1 mM EDTA, 1 mM β-mercaptoethanol, and 10% (w/v) glycerol)+0.6 M KCl. After adjusting to a volume of 50 ml with the same buffer, the mixture was stirred at 4° C. for 1 hr and centrifuged in a Sorval T865 rotor as before. The clear, straw-colored supernatant was carefully removed, frozen in dry ice, and stored at −70° C.

Phosphocellulose Chromatography.

After thawing, the second supernatant was loaded at 38 cm/hr on a 4.5 ml phosphocellulose column (Whatman P-11) equilibrated in Buffer X+0.6 M KCl and the column was washed with 5 column volumes of Buffer X+0.6 M KCl. The column was developed with a 10-column volume linear gradient of Buffer X+0.6 M KCl to Buffer X+1.7 M KCl at 19 cm/hr. Int-containing fractions eluting between 0.7 and 1.1 M KCl were pooled and stored at −70° C.

Hydroxyapatite Chromatography

The phosphocellulose pool was loaded at 38 cm/hr on a 1.5-ml hydroxyapatite column (Bio-Rad, ceramic, type II) equilibrated in Buffer X+0.6 M KCl. The pool was diluted with Buffer X to match the ionic strength of Buffer X+0.6 M KCl before loading. The column was washed with buffer X+1 M KCl. Int was eluted at 19 cm/hr with a 10-column volume linear gradient of Buffer X+0.6 M KCl to Buffer X+0.6 M KCl +0.025 M KPO₄. Int-containing fractions were pooled, BSA was added to 2 mg/ml, and the pool was frozen at −70° C.

Purification of Stimulatory Protein as a Side Fraction of a Native Int Preparation

Cells were grown and harvested and cell extract was prepared as described in the Materials and Methods section Purification of Native Int. The clarified cell extract (60 ml) was diluted to 100 ml with Buffer X (see section: Purification of Native Int) and centrifuged at 4° C. at 41,000 rpm in a Sorval T865 rotor for 200 min. The supernatant was divided into 25 ml aliquots in 50 ml conical tubes and submerged into a boiling water bath for 30 minutes. The heated suspension was centrifuged at 27,000× g for 45 minutes. The supernatant was collected and diluted with Buffer X+1.7 M KCl to match the ionic strength of Buffer X+0.6 M KCl and loaded at 15 cm/hr onto a 18 ml phosphocellulose (Whatman P-11) column (1.6×9 cm) which had been equilibrated in Buffer X+0.6 M KCl. The column was washed with 10 column volumes of Buffer X+0.6 M KCl and developed with a 10-column volume linear gradient of Buffer X+0.6 M KCl to Buffer X+1.7 M KCl. Fractions were stored at −70° C. SDS PAGE analysis of aliquots of the fractions revealed a single protein band migrating with an apparent molecular weight of 11 kDa. The protein eluted at 1.2 M KCl. Fractions containing the 11-KDa protein were pooled and diluted with Buffer X to match the ionic strength of Buffer X+0.2 M KCl. The diluted pool was loaded at 76 cm/hr onto a 1 ml Mono S column (Pharmacia) equilibrated in Buffer X+0.2 M KCl. The protein was eluted with Buffer X+1.0 M KCl. Fractions containing the peak of 11-KDa protein were pooled and stored at −70° C. The protein was subjected to amino-terminal amino acid sequence analysis as described in Materials and Methods section Amino-Terminal Amino Acid Sequence Analysis of Stimulatory Proteins and found to be ribosomal protein S20.

Purification of Stimulatory Proteins from Cells Producing Native Int

Cells were grown and harvested as described in Materials and Methods section Purification of Native Int. Cell slurry (60 g cells) was thawed at room temperature and placed on ice. A 20 mg/ml solution of lysozyme in 250 mM Tris-HCl (pH 7.4) was added in a volume 1/20 the volume of cells. After 40 minutes on ice with occasional mixing, KCl was added to a final concentration of 0.6 M. The slurry was divided into 25 ml aliquots in 50 ml conical tubes and submerged in a 72° C. water bath for 25 minutes. The suspension was spun at 27,000× g for 45 minutes. The supernatant was loaded at 15 cm/hr onto a 10 ml phosphocellulose column (Whatman P-11) (1.6×5 cm) equilibrated in Buffer X+0.6 M KCl. The column was washed with 10 column volumes of Buffer X+0.6 M KCl and developed with a 10-column volume linear gradient of Buffer X+0.6 M KCl to 1.7 M KCl. The fractions were assayed for ability to stimulate λ integrase activity (see Materials and Methods section Integrative Recombination Gel Assay). Two peaks of stimulating activity were found. Two pools were made, from fractions eluting at ˜0.8 M KCl (Pool 1) and from fractions eluting at ˜1.2 M KCl (Pool 2), and stored at −70° C.

The pools were processed separately on Mono S. Each pool was diluted with Buffer X to match the ionic strength of Buffer X+0.2 M KCl and loaded at 76 cm/hr onto a 1 ml Mono S column (Pharmacia) equilibrated with Buffer X+0.2 M KCl. The column was washed with 10 column volumes of Buffer X+0.2 M KCl and developed with a 20-column volume linear gradient of Buffer X+0.2 M KCl to Buffer X+1.7 M KCl. Fractions were stored at −70° C.

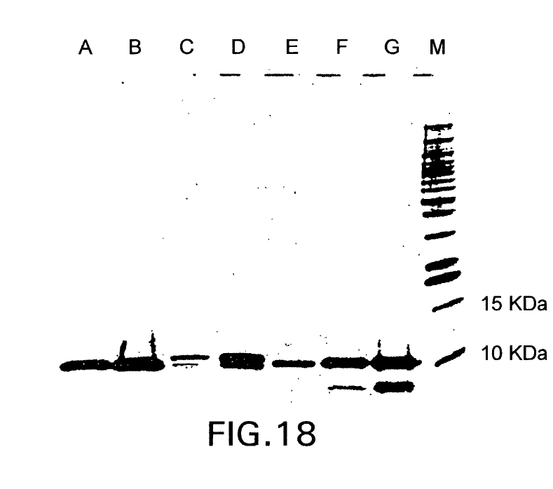

The fractions from each column were assayed for ability to stimulate λ integrase activity. Pool I from phosphocellulose was fractionated into two activity peaks by Mono S. The primary protein band in the first peak (FIG. 18, lanes A and B) was determined by N-terminal amino acid sequence analysis to be ribosomal protein L27 (see Materials and Methods section Amino-Terminal Amino Acid Sequence Analysis of Stimulatory Proteins). The second peak eluting later in the gradient was found to be composed of two major protein bands by SDS PAGE analysis (FIG. 18, lanes C and D). One protein co-migrated with L27 and the other migrated more slowly than L27 and S20 (lane E). Pool 2 from phosphocellulose was fractionated into one peak of activity by Mono S which eluted at a slightly higher salt concentration than the second peak of Pool 1 on Mono S. The main protein in this activity peak co-migrated during SDS-PAGE analysis with S20 protein (FIG. 18, lanes F and G).

Amino-Terminal Amino Acid Sequence Analysis of Stimulatory Proteins

Protein samples were subjected to SDS PAGE as described in Materials and Methods section SDS PAGE. The gel was equilibrated in transfer buffer (0.05 M Tris, 0.04 M boric acid, 0.5 mM EDTA, 20% (v/v) methanol (pH 8.4)). PVDF membrane (Immobilon P from Millipore) was prepared according to manufacturer's instructions and equilibrated in transfer buffer. The protein was transferred to the membrane using a BioRad mini blotting apparatus at 100 volts for 1 hour. The membrane was stained with Coomassie Blue R-250 staining solution and destained in 100% (v/v) methanol. The membrane was air dried and the stained protein band was excised from the membrane and stored in a 1.5-ml microcentrifuge tube.

Amino-terminal amino acid sequence analysis was performed on membrane bound protein samples by automated Edman sequence analysis by the HHMI Biopolymer Laboratory, W. M. Keck Foundation, New Haven, Conn.

Cloning of Int-His₆

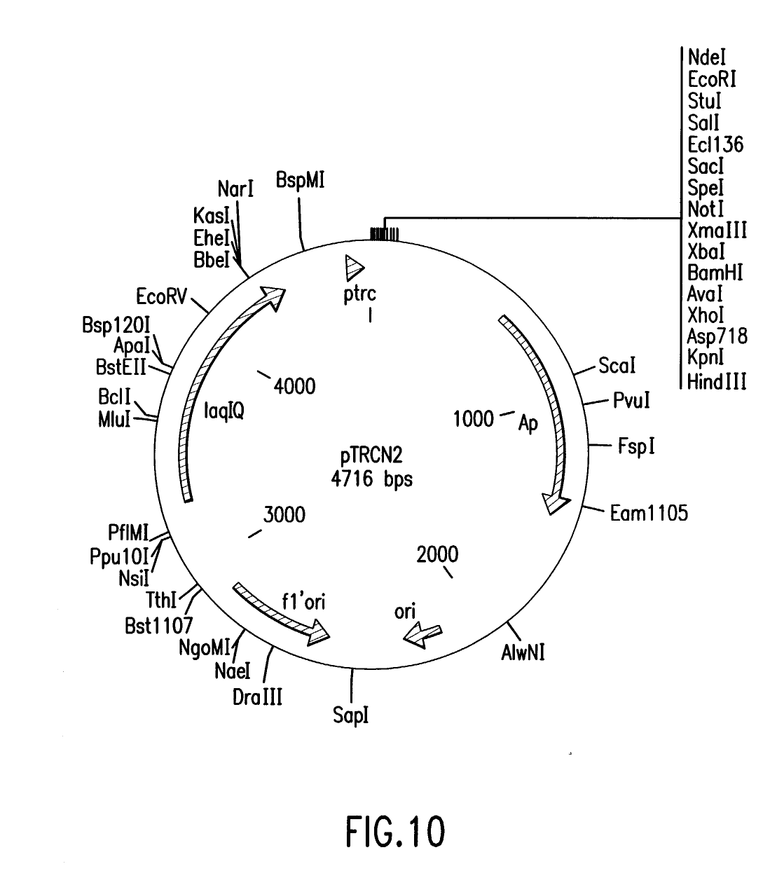

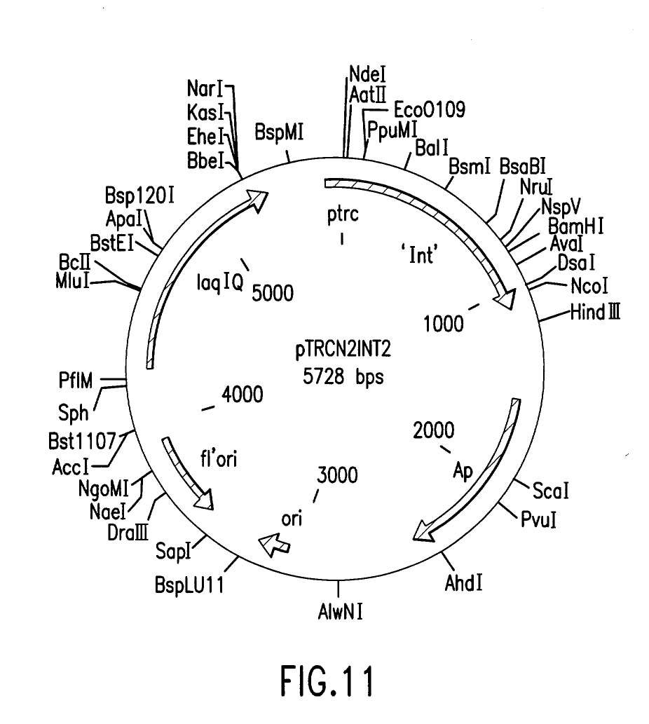

The following two oligonucleotides were used to clone the Int gene: TAT TAT TAT CAT ATG GGA CGA CGT CGA AGT CAT GAG CGC CGG GAT (SEQ ID NO:9) and A TTA TTA AGC TTA TTA ATG GTG ATG ATG GTG ATG TTT GAT TTC AAT TTT GTC CCA CTC (SEQ ID NO:10). The oligonucleotides were used to generate a 1,092-bp PCR amplification product using X DNA as the template. DNA was amplified (Materials and Methods section PCR) during 8 cycles composed of the following steps: 95° C. for 15 seconds, 55° C. for 15 seconds, and 72° C. for 90 seconds. The 1,092-bp PCR product was digested with NdeI and HindIII and cloned into the NdeI and HindIII sites of plasmid pTRCN2 (FIG. 10) in an E. coli DH10B host. This construct is called pTRCN2INT2 (FIG. 11). The Int gene is under control of a pTRC promoter and contains a sequence coding for a His₆ tag at the carboxy end of the protein. The DNA sequence of the Int gene in pTRCN2INT2 was determined and found to match the published sequence, except as modified below. Arg codons AGA and AGG originally coding for Arg at positions 3 and 4 were changed to CGA and CGT, respectively, which are Arg codons more frequently used in E. coli.

Purification of Int-His₆

Int-His₆ was purified from E. coli DH10B cells bearing plasmid pTRCN2INT2 (see Materials and Methods section Cloning of Int-His₆).

Growth of Cells.

To prepare seed stocks, E. coli DH10B cells bearing plasmid pTRCN2INT2 were grown at 30° C. in Buffered Rich medium+100 μg/ml ampicillin to an A₅₉₀˜2. Culture was mixed 1:1 with 50% glycerol. The mixture was aliquoted by 1 ml into cryovials on ice and then stored at −80° C.

For a small scale growth, cells from a frozen glycerol stock were inoculated into 2×50 ml Buffered Rich medium+100 μg/ml ampicillin in 2×250-ml bottom-baffled shake flasks. Cells were grown for 16.5 hours at 30° C. and 250 rpm to an A₅₉₀ of ˜4.0. Twenty-five ml of the primary shake flask growth was used to inoculate each of 4, 2.8-L bottom-baffled Fernbach flasks containing 1 L of Buffered Rich medium +100 μg/ml ampicillin (for an initial A₅₉₀ of ˜0.1). Cultures were grown at 30° C. until an A₅₉₀=1.0 to 1.5 was achieved. The cultures were induced by adding IPTG to 1 mM. Growth was continued for 2 hr at 30° C. The culture was chilled by icing in 4×1 L centrifuge bottles and harvested by centrifugation at 4,500 rpm (5,895× g) and 4° C. for 12 minutes. Each pellet was washed by resuspension in ˜7 ml 50 mM Tris-HCl (pH 8.0), 100 mM NaCl at 4° C. and re-spun. The pellets were frozen and stored at −80° C.

For a large scale growth, 50 ml of Buffered Rich medium +100 μg/ml ampicillin in a 250 ml bottom baffled shake flask was inoculated with 1 ml of a frozen seed. Cells were grown at 30° C. and 250 rpm to an A₅₉₀ of 0.8 to 1.2. The entire 50 ml was inoculated into 500 ml Buffered Rich medium +100 μg/ml in a 2.8-L bottom-baffled Fernbach. Growth was continued at 30° C. and 250 rpm to an A₅₉₀=0.8 to 1.2.

10 L of Buffered Rich medium+100 μg/ml ampicillin in a 14-L vessel was inoculated with all 500 ml of culture. Temperature was maintained at 30° C. Dissolved oxygen levels were controlled at >30% and pH at 7+/−0.3. At A₅₉₀=1.5 to 2.0 the culture was induced by adding IPTG to 1 mM. Growth was continued for 2 hr at 30° C. The vessel was chilled and harvested by centrifugation in a Sharples centrifuge. Cell paste was frozen and stored at −80° C.

Purification

Frozen cells (20 g) were thawed on ice and suspended in 40 ml of Tris-HCl (pH 8.0)-10% (w/v) sucrose. Cells were disrupted on ice by sonication (4, 30 second bursts at 70% maximum setting), and the extract was centrifuged at 27,000× g for 30 minutes at 4° C. The supernatant was collected. The supernatant was mixed with 20 ml (packed volume) of Chelating Sepharose (Pharmacia) charged with NiSO₄ and equilibrated with Buffer A (50 mM Tris-HCl (pH 8.0), 0.3 M NaCl, 10% (v/v) glycerol). The slurry was transferred to 50-ml conical tubes and was gently rocked for 30 minutes at 4° C. The slurry was then packed into a 1.6 cm column and attached to an FPLC system (Pharmacia). The column was washed with 20 column volumes of Buffer A+20 mM Imidazol at 30 cm/hr. The protein was eluted with a 15-column volume linear gradient from Buffer A+20 mM Imidazol to Buffer A+500 mM Imidazol. Fractions were analyzed by SDS PAGE. Fractions containing Int-His₆ were pooled and 0.5 M EDTA was added to a final concentration of 1 mM. The pool was then transferred to 10,000 molecular weight cut off (MWCO) dialysis tubing and dialyzed against 50 volumes of Buffer B (50 mM Tris-HCl (pH 7.5), 1 mM EDTA, 10% (v/v) glycerol, and 1 mM β-mercaptoethanol). The dialyzed pool was loaded at 38 cm/hr onto a 2 ml (1×1 cm) EMD-SO₄ (EM Separations) column equilibrated in Buffer B+0.2 M NaCl. The column was washed with 10 column volumes of Buffer B+0.2 M NaCl at 76 cm/hr and developed with a 15-column volume linear gradient from Buffer B+0.2 M NaCl to Buffer B+1.6 M NaCl. Int-His₆ eluted at approximately 1.1 M NaCl based upon analysis by SDS PAGE. The peak fractions were pooled and the pool was transferred to 10,000 MWCO dialysis tubing and dialyzed against 100 volumes of Buffer C (Buffer B minus EDTA). The dialyzed pool was loaded at 38 cm/hr onto a 1 ml (0.5×1 cm) hydroxyapatite column (Type II, BioRad) equilibrated in Buffer C. The column was washed with 10 column volumes of Buffer C+1 M NaCl and developed with 10 column volumes of Buffer C+0.6 M NaCl +25 mM KPO₄ at 19 cm/hr. The fractions were analyzed by SDS PAGE and the peak fractions containing Int-His₆ were pooled. The pool was transferred to 10,000 MWCO dialysis tubing and was dialyzed against 200 volumes of 50 mM Tris-HCl (pH 7.5), 50 mM NaCl, 0.05 mM EDTA, 50% (v/v) glycerol, and 1 mM DTT overnight at 4° C. The final sample was stored at −70° C.

Cloning of Xis-His₆

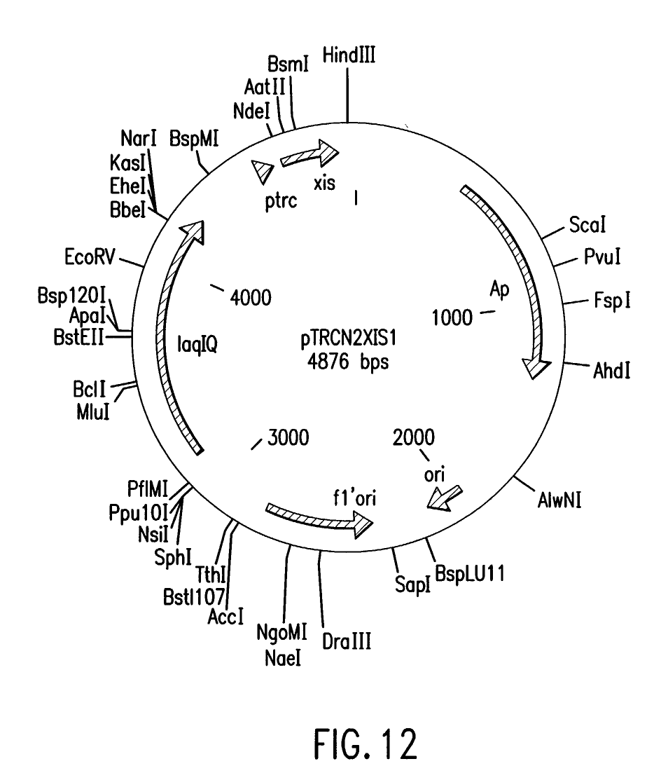

The following two oligonucleotides were used to clone the Xis gene: TAT TAT TAT CAT ATG TAC TTG ACA CTT CAG GAG (SEQ ID NO:11) and ATT ATT AAG CTT ATT AAT GGT GAT GAT GGT GAT GTG ACT TCG CCT TCT TCC CAT T (SEQ ID NO:12). The oligonucleotides were used to generate a 219-bp PCR product using X DNA as the template. DNA was amplified (Materials and Methods section PCR) during 15 cycles composed of the following steps: 95° C. for 15 seconds, 55° C. for 15 seconds, and 72° C. for 60 seconds. The 219-bp PCR product was digested with NdeI and HindIII and cloned into the NdeI and HindIII site of pTRCN2 (FIG. 10). The resulting construct was called pTRCN2XIS1 (FIG. 12). The Xis gene is under control of a pTRC promoter and contains a sequence coding for a His₆ tag at the carboxy end of the protein. The DNA sequence of the Xis gene in pTRCN2XIS1 was determined and found to match the published sequence.

Purification of Xis-His₆

Xis-His₆ was purified from E. coli Stbl 2 cells bearing plasmid pTRCN2XIS 1 (see Materials and Methods section Cloning of Xis-His₆).

Growth of Cells

To prepare seed stocks, E. coli Stbl 2 cells bearing plasmid pTRCN2XIS1 were grown at 37° C. in Buffered Rich medium+100 μg/ml ampicillin to an A₅₉₀˜3. Culture was mixed 1:1 with 50% glycerol. The mixture was aliquoted by 1 ml into cryovials on ice and then stored at −70° C.

For small scale growths, cells from a frozen glycerol stock were inoculated into 50 ml Buffered Rich medium+100 μg/ml ampicillin in a 250-ml bottom-baffled shake flask. Cells were grown for 17 hours at 37° C. and 250 rpm to an A₅₉₀ of ˜4.0.

12 ml of the primary shake flask growth was used to inoculate each of 4, 2.8-L bottom-baffled Fernbach flasks containing 1 L of Buffered Rich medium+100 μg/ml ampicillin (for an initial A₅₉₀ of ˜0.05). Cultures were grown at 37° C. until an A₅₉₀=1.5 to 2.0 was achieved. The cultures were induced by adding IPTG to 1 mM. Growth was continued for 2 hr at 37° C. The culture was chilled by icing in 4×1 L centrifuge bottles and harvested by centrifugation at 4,500 rpm (5,895× g) and 4° C. for 15 minutes. Each pellet was washed by resuspension in ˜20 ml used medium and re-spun. The pellets were frozen and stored at −70° C.

For a large scale growth, a 50 ml culture of Buffered Rich medium+100 μg/ml ampicillin in a 250-ml bottom baffled shake flask was inoculated with 1 ml of a frozen seed. Cells were grown at 37° C. and 250 rpm to an A₅₉₀ of 0.6 to 1.4. The entire 50 ml was inoculated into 500 ml Buffered Rich medium+100 μg/ml ampicillin in a 2.8-L bottom-baffled Fernbach. Growth was continued at 37° C. and 250 rpm to an A₅₉₀=0.6 to 1.4. Ten L of Buffered Rich medium+100 μg/ml ampicillin in a 14-L vessel was inoculated with all 500 ml of culture. Temperature was maintained at 37° C. Dissolved oxygen levels were controlled at >30% and pH at 7+/−0.3. At A₅₉₀=1.5 to 2.0 the culture was induced by adding IPTG to 1 mM. Growth was continued for 2 hr at 37° C. The vessel was chilled and harvested by centrifugation in a Sharples centrifuge. Cell paste was frozen and stored at −70° C.

Purification.

Frozen cells (20 g) were thawed on ice and suspended in 20 ml of 50 mM Tris-HCl (pH 8.0), 10% (w/v) sucrose, 0.002 mg/ml leupeptin, 0.002 mg/ml pepstatin A, 0.8 mg/ml benzamide, and 0.05 mg/ml Pefablock. Cells were disrupted by sonication (5 second bursts at 80% of the maximum setting alternated with 5 seconds off for 3 minutes). The extract was centrifuged at 27,000× g for 30 minutes at 4° C. and the supernatant was collected. The supernatant was loaded at 30 cm/hr onto a 20-ml column (1.6×10 cm) of Chelating Sepharose (Pharmacia) charged with NiSO₄ and equilibrated with Buffer D (50 mM Tris-HCl (pH 7.5), 0.4 M NaCl, and 10% (v/v) glycerol)+5 mM Imidazol. The column was washed with 20 column volumes of Buffer D+5 mM Imidazol at 30 cm/hr and developed with a 15-column volume linear gradient from Buffer D+5 mM Imidazol to Buffer D+450 mM Imidazol at 12 cm/hr. Fractions were analyzed by SDS PAGE. Peak fractions containing the Xis-His₆ protein were pooled and 0.5 M EDTA and 1 M DTT were added to final concentrations of 1 mM and 4 mM, respectively. he pool was then loaded at 38 cm/hr onto a 5.5 ml (1.0×7.0 cm) EMD-SO₄ (EM Separations) column equilibrated in Buffer E (50 mM Tris-HCl (pH 7.5), 1 mM EDTA, 10% (v/v) glycerol, and 4 mM DTT)+0.4 M NaCl. The column was washed with 10 column volumes of Buffer E+0.4 M NaCl at 76 cm/hr and developed with a 10-column volume linear gradient from Buffer E+0.4 M NaCl to Buffer E+2 M NaCl at 15 cm/hr. Fractions were analyzed by SDS PAGE. Xis-His₆ elutes in a broad peak at approximately 1.1-1.8 M NaCl. The peak fractions containing Xis-His₆ were pooled. The pool was diluted with Buffer E to match the ionic strength of Buffer E+0.2 M NaCl and loaded at 152 cm/hr onto a 1 ml (0.5×5.0 cm) Mono S (Pharmacia) column equilibrated in Buffer E+0.2 M NaCl. The column was washed with 10 column volumes of Buffer E+0.2 M NaCl. Xis-His₆ was eluted with 10 column volumes of Buffer E+2.0 M NaCl at 61 cm/hr. Fractions were analyzed by SDS PAGE and the peak fractions containing Xis-His₆ were pooled. The pool was transferred to a 2,000 molecular weight cut off dialysis cassette (Pierce) and was dialyzed against 200 volumes of 50 mM Tris-HCl (pH 7.5), 50 mM NaCl, 0.05 mM EDTA, 50% (v/v) glycerol, and 1 mM DTT overnight at 4° C. The final sample was stored at −70° C.

Cloning of S20

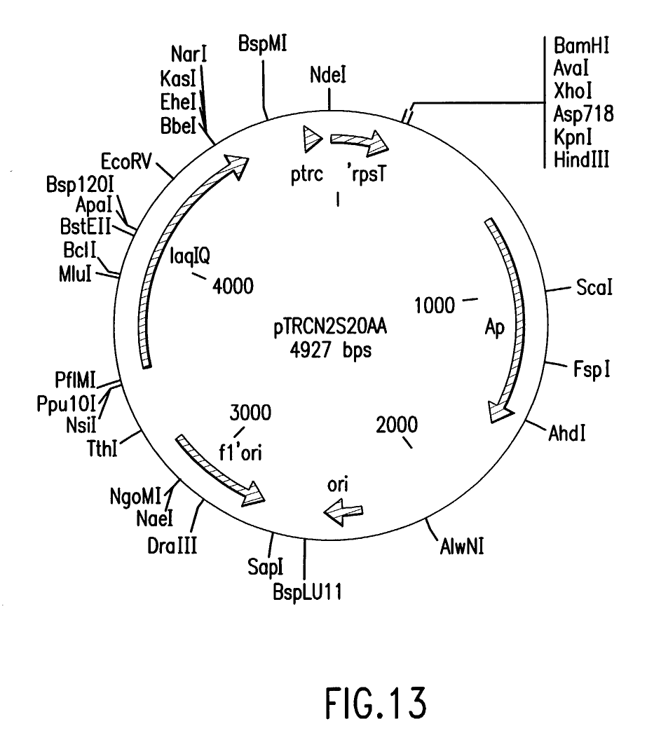

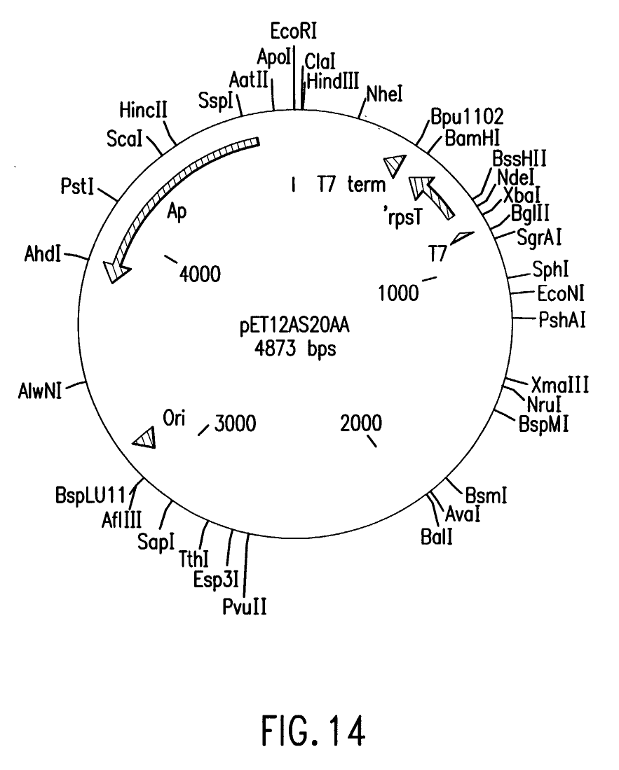

The following two oligonucleotides were used to clone the S20 gene: TAT TAT TAT CAT ATG GCT AAT ATC AAA TCA GCT AAG (SEQ ID NO:13) and ATT ATT GGA TCC ATT AAG CCA GTT TGT TGA TCT (SEQ ID NO:14). The oligonucleotides were used to generate a 267-bp PCR product using E. coli chromosomal DNA as template. DNA was amplified (Materials and Methods section PCR) during 15 cycles composed of the following steps: 95° C. for 15 seconds, 50° C. for 15 seconds, and 67° C. for 30 seconds. The 267-bp PCR product was digested with NdeI and BamHI and cloned into the NdeI and BamHI sites of pTRCN2 (FIG. 10) in E. coli DH10B. The resulting construct was called pTRCN2S20AA (FIG. 13). The S20 gene is under control of a pTRC promoter. The DNA sequence of the S20 gene in pTRCN2S20AA was determined and found to match the published sequence, except as noted below. The initiation codon was changed from TTG to ATG during cloning to enhance expression. pTRCN2S20AA was digested with NdeI and BamHI to generate a 267-bp fragment that was cloned into the NdeI and BamHI sites of pET12A (Novagen) in E. coli strain BL21DE3. The resulting construct was called pET12AS20AA (FIG. 14). The S20 gene is under control of a T7 promoter.

Purification of Recombinant S20

S20 was purified from E. coli BL21DE3 bearing plasmid pET12AS20AA (see Materials and Methods section Cloning of S20).

Growth of Cells.

Cells from a glycerol stock of BL21DE3 bearing plasmid pET12AS20AA were inoculated into 3 ml of LB broth containing 100 μg/ml ampicillin. This inoculum was diluted into LB broth+100 μg/ml ampicillin 1:100 and the 300-ml culture was grown overnight at 30° C. The A₆₅₀ of the culture should not exceed 1.0. This culture was used to inoculate 10 flasks containing 500 ml each of Circlegrow (BIO 101) plus 100 μg/ml ampicillin plus 1 mM MgSO₄. Cells were grown at 37° C. until the A₆₅₀ was 0.5 and expression of S20 was induced by the addition of IPTG to 0.5 mM. After growth at 37° C. for 4 hours, cells were harvested by centrifugation at 4° C. and stored at −70° C.

Purification

Frozen cells (10 g) were thawed on ice and suspended in 25 ml of 50 mM Tris-HCl (pH 7.5), 0.2 mM EDTA, 10% (v/v) glycerol, 0.2 mM DTT, 0.2 μg/ml leupeptin, and 1 mM PMSF. Cells were then disrupted by sonication (5 second bursts at 80% of the maximum setting alternated with seconds off for 1.5 minutes). NaCl (5.0 M) was then added to a final concentration of 0.67 M. The slurry was mixed by inverting the container and then placed on ice for 10 minutes. The mixture was centrifuged at 27,000× g for 30 minutes at 4° C. and the supernatant was collected. The supernatant was diluted with Buffer B (50 mM Tris-HCl (pH 7.5), 1 mM EDTA, 10% (v/v) glycerol, 1 mM β-mercaptoethanol) to match the ionic strength of Buffer B+0.3 M NaCl and then loaded at 30 cm/hr onto a 7.5 ml (1.8×3.7 cm) EMD-SO₄ (EM Separations) column equilibrated in Buffer B+0.3 M NaCl. The column was washed with 10 column volumes of Buffer B+0.3 M NaCl at 30 cm/hr and developed with a 15-column volume linear gradient from Buffer E+0.3 M NaCl to Buffer E+1.8 M NaCl at 30 cm/hr. Fractions were analyzed by SDS PAGE. S20 eluted at approximately 0.9 M NaCl. The fractions containing the peak of S20 were pooled. The pool was transferred to a 2,000 molecular weight cut off dialysis cassette (Pierce) and dialyzed against 200 volumes of 50 mM Tris-HCl (pH 7.5), 50 mM NaCl, 0.05 mM EDTA, 50% (v/v) glycerol, and 1 mM DTT overnight at 4° C. The final sample was stored at −70° C.

Integrative Recombination Gel Assay

Reaction mixtures (10 μl final volume) for monitoring integrative recombination (defined as containing linearized attB and supercoiled attP DNA substrates) by agarose gel electrophoresis were incubated at 25° C. for 45 minutes. Reactions were initiated by adding 1 μl of Int or Int-His₆ (contained in 50 mM Tris-HCl (pH 7.5), 1 mM EDTA, 600 mM KCl, 2 mg/ml BSA, and 10% (v/v) glycerol) plus or minus potential stimulatory proteins to a mixture containing 20 mM Tris-HCl (pH 8.0), 5 mM spermidine, 50 μg/ml BSA, 125 ng linearized pBB105, 125 ng supercoiled pHN894, and 12.5 ng IHF. Incubation was stopped by raising the temperature to 70° C. for 10 minutes and then adding 2.5 μl of 25% (w/v) Ficoll 400, 0.5% (w/v) SDS, and 0.00625% (w/v) bromophenol blue. In some cases, reaction mixtures were treated with proteinase K (10 to 20 μg at 25° C. for 15 minutes). Samples were analyzed by electrophoresis in a 1% agarose minigel cast in 40 mM Tris-acetate (pH 8.3), 1 mM EDTA (TAE) and 1 μg/ml ethidium bromide and run in TAE at 105 V for 30 minutes. Recombination activity is indicated by the appearance of a DNA band migrating at 10,201 bp. A unit of Int activity was defined as described (Nash, H. A. Methods Enz. 100: 210-216 (1983)).

Excisive Recombination Gel Assay

Reaction mixtures (10 μl final volume) for monitoring excisive recombination (defined as containing linearized attL and supercoiled attR DNA substrates) by agarose gel electrophoresis were incubated at 25° C. for 45 minutes. Reactions were initiated by adding 1 μl of Int or Int-His₆ (contained in 50 mM Tris-HCl (pH 7.5), 1 mM EDTA, 600 mM KCl, 2 mg/ml BSA, and 10% (v/v) glycerol) plus or minus potential stimulatory proteins to a mixture containing 20 mM Tris-HCl (pH 8.0), 5 mM spermidine, 50 μg/ml BSA, 125 ng linearized pHN872, 125 ng supercoiled pHN868, 12.5 ng IHF, and 28 ng Xis or Xis-His₆. Incubation was stopped by raising the temperature to 70° C. for 10 minutes and then adding 2.5 μl of 25% (w/v) Ficoll 400, 0.5% (w/v) SDS, and 0.00625% (w/v) bromophenol blue. In some cases, reaction mixtures were treated with proteinase K (10 to 20 μg at 25° C. for 15 minutes). Samples were analyzed by electrophoresis in a 1% agarose minigel cast in 40 mM Tris-acetate (pH 8.3), 1 mM EDTA (TAE) and 1 μg/ml ethidium bromide and run in TAE at 105 V for 30 minutes. Recombination activity is indicated by the appearance of a DNA band migrating at 9,991 bp.

Integrative Recombination Colony-Forming Assay

Reaction mixtures (20 μl final volume) for monitoring integrative recombination (defined as containing linearized attB and supercoiled attP DNA substrates) by transformation of E. coli were incubated at 25° C. for 45 minutes. Reactions were initiated by adding 4 μl of Int or Int-HiS6 (contained in 50 mM Tris-HCl (pH 7.5), 50 mM NaCl, 1 mM EDTA, 200 μg/ml BSA, and 50% (v/v) glycerol) plus or minus S20 to a mixture containing 50 mM Tris-HCl (pH 7.5), 50 mM NaCl, 2.5 mM spermidine, 0.25 mM EDTA, 200 μg/ml BSA, 100 ng linearized pEZC7501, 100 ng supercoiled pEZ13835, and 10 ng IHF. Incubation was stopped by raising the temperature to 70° C. for 10 minutes. Proteinase K (4 μg in 1 μl) was added and after 10 minutes at 37° C. the mixture was centrifuged (14,000 rpm for 30 seconds). The mixture (1 μl) was used to transform 100 μl of ME DH5α E. coli competent cells (LTI) in a sterile polypropylene tube on ice. After 30 minutes on ice, the tube was heat shocked in a 42° C. water bath for 45 seconds. The tube was then placed on ice for 2 minutes. S.O.C. medium (0.9 ml) was added to the tube, and the tube was placed in a shaker for 60 minutes at 37° C. and 225 rpm. Aliquots (10 and 100 μl) of the transformed cells were spread on separate agar plates prepared in LB medium +100 μg/ml kanamycin, and the plates were incubated at 37° C. for 16 to 24 hours. Kanamycin-resistant colonies arise only as the result of an integrative recombination event.

Excisive Recombination Colony-Forming Assay

Reaction mixtures (20 μl final volume) for monitoring excisive recombination (defined as containing linearized attR and supercoiled attl DNA substrates) by transformation of E. coli were incubated at 25° C. for 45 minutes. Reactions were initiated by adding 4 μl of Int or Int-His₆ (contained in 50 mM Tris-HCl (pH 7.5), 50 mM NaCl, 1 mM EDTA, 200 μg/ml BSA, and 50% (v/v) glycerol) plus or minus S20 to a mixture containing 50 mM Tris-HCl (pH 7.5), 50 mM NaCl, 2.5 mM spermidine, 0.25 mM EDTA, 200 μg/ml BSA, 100 ng linearized pEZC8402, 100 ng supercoiled pEZ11104, 12.5 ng IHF, and 28 ng Xis or Xis-His₆. Incubation was stopped by raising the temperature to 70° C. for 10 minutes. Proteinase K (4 μg in 1 μl) was added and after 10 minutes at 37° C. the mixture was centrifuged (14,000 rpm for 30 seconds). A portion of the reaction mixture (10.5 μl) was diluted with 89.5 μl of T₁₀E₁. The diluted mixture (1 μl) was used to transform 100 μl of ME DH5α E. coli competent cells (LTI) in a sterile polypropylene tube on ice. After 30 minutes on ice, the tube was heat shocked in a 42° C. water bath for 45 seconds. The tube was then placed on ice for 2 minutes. S.O.C. medium (0.9 ml) was added to the tube, and the tube was placed in a shaker for 60 minutes at 37° C. and 225 rpm. Aliquots (10 and 100 μl) of the transformed cells were spread on separate agar plates prepared in LB medium +100 μg/ml ampicillin, and the plates were incubated at 37° C. for 16 to 24 hours. Ampicillin-resistant colonies arise only as the result of an excisive recombination event.

### Results

Part I: Restoration of Integrase Activity by Mixing with Cell Extract Components

Restoration of Int Activity by Column Fractions

Purification of Int overexpressed in E. coli involved differential salt precipitation followed by phosphocellulose and hydroxyapatite chromatography (Materials and Methods). When an attempt was made to purify native Int by this procedure, it was found that Int integrative recombination activity (determined as described in Materials and Methods section, Integrative Recombination Gel Assay) was maintained through the phosphocellulose chromatography step, but was lost during the final hydroxyapatite chromatography step. No activity was found in any hydroxyapatite column fraction. This was not caused by loss of Int protein during chromatography, since SDS-PAGE analysis of the hydroxyapatite fractions revealed the presence of a single protein of molecular weight 40 kDa, consistent with the bound protein being Int. Fractions containing the peak of the 40-KDa protein were pooled and the pool was assayed for integrative recombination activity. As the results shown in Table 2 indicate, no activity was observed.

TABLE 2
SUMMARY OF PURIFICATION OF NATIVE Int

| | | Total Protein | Specific Activity |
|---|---|---|---|
| Purification Step | Total Units | (mg) | (U/mg) |
| Crude Extract | 228,000 | 1,294 | 176 |
| Differential salt | 67,000 | 153 | 441 |
| precipitation | | | |
| Phosphocellulose | 21,000 | 6.7 | 3,134 |
| Hydroxyapatite | 0 | 0.2 | — |
| Hydroxyapatite + | ˜30,000 | 0.2 | ˜150,000 |
| stimulatory | | | |
| protein(s) | | | |

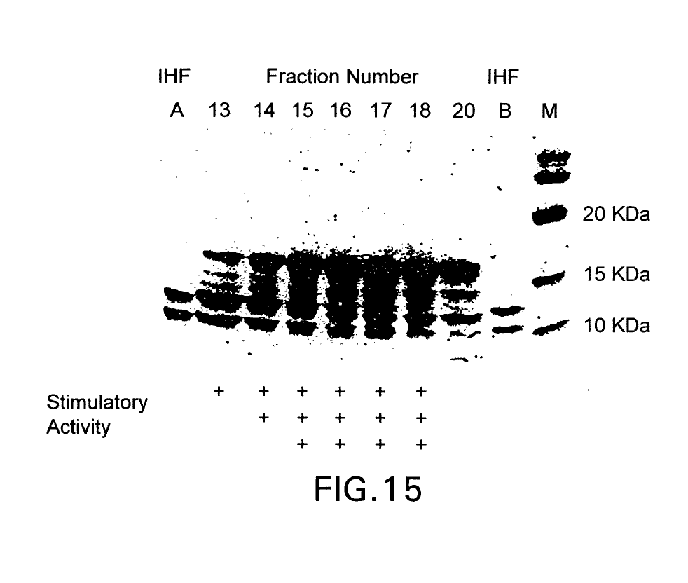

Examination of the proteins in the phosphocellulose pool by SDS PAGE revealed the presence of Int (40 kDa) and a number of smaller proteins (at least six) in the 5 to 17 kDa range. E. coli DNA binding proteins that stimulate Int activity, such as HU, fall in this small size range (Segall, A. M. et. al, EMBO J. 13: 4536-4548 (1994)). From the above it was hypothesized that this preparation of Int required additional component(s) for activity beyond the IHF already present in recombination reaction mixtures (Materials and Methods). Further, the chromatography results suggested that this component(s) coeluted with Int from phosphocellulose, but was not bound by hydroxyapatite. To test this hypothesis, the material from the original phosphocellulose pool that did not bind to hydroxyapatite was fractionated again on a phosphocellulose column. Samples from fractions from this column were assayed for ability to restore integrative recombination activity to the inactive Int pooled from the hydroxyapatite column. It was found that fractions eluting from the phosphocellulose column at around 1.0 M KCl contained a component(s) that restored recombination activity to the inactive Int (FIG. 15). The fractions with the greatest stimulatory activity (Fraction Numbers 15 through 18 in FIG. 15) were used for further characterization. Unit assay of the Int hydroxyapatite pool in the integrative recombination assay in the presence of an optimal amount of this stimulatory material indicated that greater than 100% of the Int activity present in the phosphocellulose pool was present in the hydroxyapatite pool when the stimulatory component(s) was present in the unit assay (Table 2).

Characterization of the Stimulatory Component(s).

SDS PAGE analysis of the stimulatory fractions from the second phosphocellulose column showed multiple small protein bands, two of which appeared similar in size to the subunits of authentic IHF (FIG. 15). On the chance that the concentration of IHF being used in the integrative recombination gel assay was not optimal, a careful titration of IHF was carried out with inactive Int in the presence and absence of stimulatory material from the phosphocellulose column. It was found that no amount of IHF alone, from 12.5 to 1,250 ng, stimulated inactive Int. In contrast, the combination of IHF at 12.5 ng and the component(s) from the phosphocellulose column did restore Int activity.

Treatment of the stimulatory component(s) with DNaseI or RNaseA did not diminish ability to stimulate Int. Placing the component(s) in a boiling water bath for 30 minutes also had no effect. However, treatment with proteinase K eliminated ability to stimulate, indicating the stimulatory component(s) was protein that could withstand high temperature.

Part II: Purification and Identification of the Stimulatory Proteins

Purification From a Side Fraction

Steps were taken to identify the protein(s) in extracts of E. coli expressing native Int that stimulate its recombinase activity. Purification was monitored by detecting the presence of Int stimulatory protein using the integrative recombination gel assay (Materials and Methods) and inactive Int, purified as just described (Materials and Methods section Purification of Native Int and Results section PART I: Restoration of Integrase Activity by Mixing with Cell Extract Components). The fact that extracts could be heated to boiling water temperatures without affecting adversely the stimulatory activity was taken advantage of. Heating served several purposes. First, any active Int present during early purification steps would be irreversibly inactivated, eliminating interference in the gel recombination assay. Second, many E. coli proteins in crude extracts precipitate at high temperature; thus heating facilitates purification of those proteins that remain soluble.

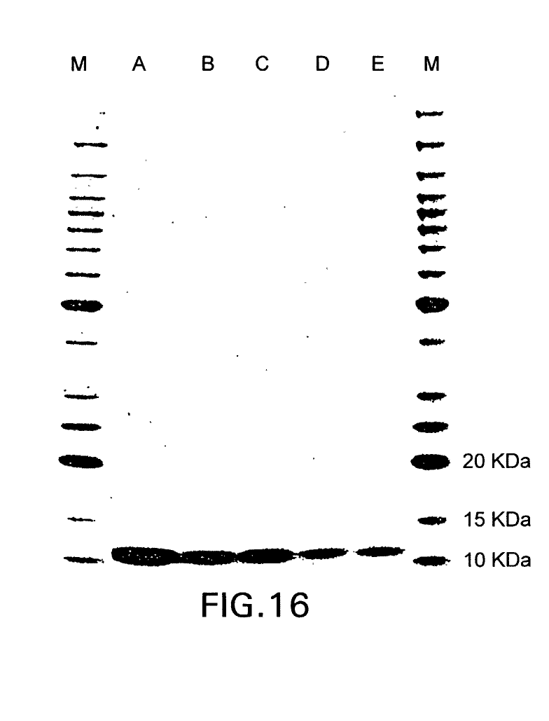

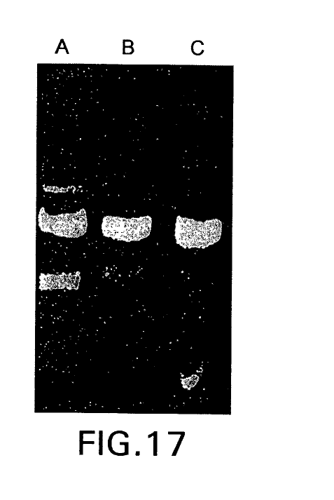

The side fractions generated early in the native Int purification (Materials and Methods section Purification of Native Int) were heated to 100° C., clarified by centrifugation, and assayed for ability to stimulate inactive Int. The supernatant from the first high speed centrifugation in the differential salt precipitation step was found to have the most stimulatory activity. Using this supernatant as starting material, a stimulatory protein was purified as described in Materials and Methods section Purification of Stimulatory Protein as a Side Fraction of a Native Int Preparation. A near homogeneous 11-KDa protein was purified after two column chromatography steps (FIG. 16) that stimulated inactive Int in the gel recombination assay (FIG. 17).

The 11-KDa protein was sent to the HHMI Biopolymer Laboratory, W. M. Keck Foundation, for amino terminal amino acid sequence analysis (Materials and Methods section Amino-Terminal Amino Acid Sequence Analysis of Stimulatory Proteins). The sequence was found to be Ala-Asn-Ile-Lys-Ser-Ala-Lys-Lys-Arg-Ala-Ile-Gln-Ser-Glu (SEQ ID NO:15). Search of the GenBank sequence data base revealed that this sequence matches amino acids 2 through 15 of E. coli 30S ribosomal protein S20 (Mackie, G. A., J. Biol. Chem. 256:8177-8182 (1981)). S20 is a very basic protein of 86 amino acids. In E. coli, S20 appears to be involved in association of the 30S ribosomal subunit with the 50S subunit and in formation of the 30S subunit translation initiation complex with fMet-tRNA and mRNA (Gotz, F. et. al Biochim. Biophys. Acta 1050: 93-97 (1990)). The gene for S20 was cloned, overexpressed, and purified (see Materials and Methods sections Cloning of S20 and Purification of Recombinant S20). The ability of recombinant S20 to stimulate Int was tested (see Results, PART II).

Purification From Total Cell Extract

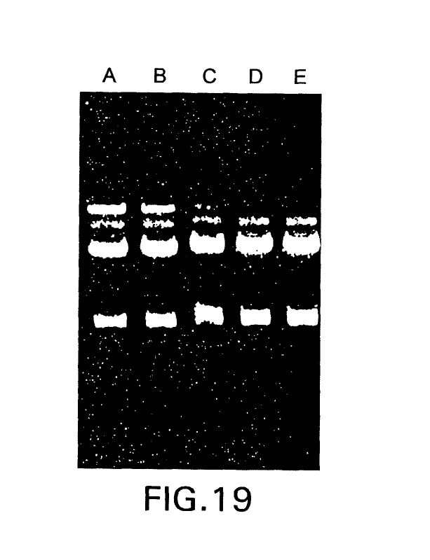

Since one small, heat resistant, nucleic acid binding protein in extracts of E. coli that stimulates Int activity was identified, an attempt was made to identify others. Using the gel recombination assay with inactive Int to assay for stimulation of Int, and starting with total E. coli cell extract, purification of stimulatory activity was repeated (see Materials and Methods section Purification of Stimulatory Proteins from Cells Producing Native Int). Again, phosphocellulose followed by Mono S chromatography was used to fractionate heated E. coli extract. A second stimulatory protein was identified that migrated on SDS PAGE slightly faster than S20 (FIG. 18). This protein was also sent to the HHMI Biopolymer Laboratory, W. M. Keck Foundation, for sequence analysis. The sequence was found to be Ala-His-Lys-Lys-Ala-Gly-Gly-Ser-Thr-Arg-Asn (SEQ ID NO:16). Search of the GenBank sequence data base revealed that this sequence matches amino acids 2 through 12 of E. coli 50S ribosomal protein L27 (Jeong, J. H. et. al, DNA Seq. 4:59-67 (1993)). L27 is a very basic protein of 85 amino acids. The proteins in fraction 18 (lanes A and B of FIG. 18), the primary constituent of which was L27, were tested for ability to stimulate Int in the integrative recombination gel assay. FIG. 19 shows that these proteins stimulated Int in the recombination assay. However, 10 times more L27 than S20 was required to produce a discernible recombinant DNA product.

Part III: Cloning of S20 and Demonstration of Activity

Cloning, Overexpression, and Purification of rS20

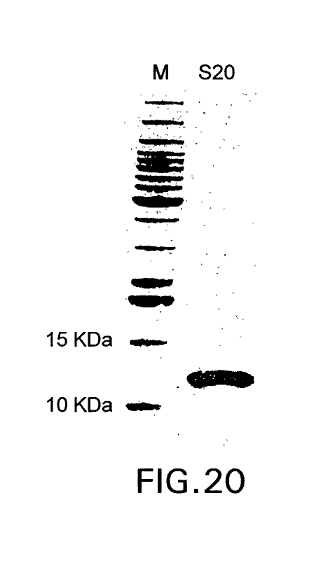

The gene for S20 from E. coli DNA was cloned under control of a T7 promoter using PCR (see Materials and Methods section Cloning of S20). The recombinant S20 was highly overexpressed and easily purified by EMD-SO₄ chromatography (see Materials and Methods section Purification of Recombinant S20). Approximately 110 mg of near homogeneous recombinant S20 (FIG. 20) was purified from 9 g of E. coli.

Characterization of rS20

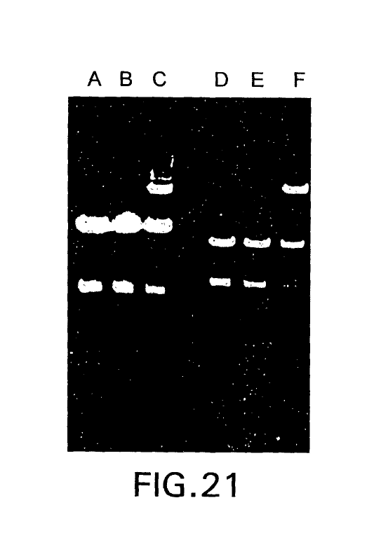

Recombinant S20 stimulated integrative and excisive λ recombination catalyzed by native Int as determined by gel assay (FIG. 10), and recombinant S20 also stimulated both integrative and excisive λ recombination catalyzed by recombinant Int-His₆ as determined both by gel assay (FIG. 21) and colony-forming assay (Tables 3 and 4). These results confirmed those obtained with native S20; that is, recombinant S20 stimulates the recombinase activity of Int.

TABLE 3
STIMULATION OF INT-HIS₆ BY RECOMBINANT S20
IN AN INTEGRATIVE RECOMBINATION COLONY-
FORMING ASSAY*

| Amt. of Recombinant S20 (ng) | Number of Colonies Formed |
|---|---|
| 0 | 35 |
| 313 | 82 |
| 625 | 255 |
| 1,250 | 233 |
| 2,500 | 5 |

*See Materials and Methods for details of assay. All reaction mixtures contained 176 ng Int-His₆ and 10 ng IHF.

TABLE 4
STIMULATION OF INT-HIS₆ BY RECOMBINANT S20
IN AN EXCISIVE RECOMBINATION COLONY-
FORMING ASSAY*

| Amt. of Recombinant S20 (ng) | Number of Colonies Formed |
|---|---|
| 0 | 9 |
| 158 | 86 |
| 313 | 1,392 |
| 625 | 83 |
| 1,250 | 23 |

*See Materials and Methods for details of assay. All reaction mixtures contained 176 ng Int-His₆, 12.5 ng IHF, and 28 ng Xis-His₆.

The order of addition of S20 and Int to a reaction appears to be important. Int should be mixed with S20 and the proteins added as a mixture to IHF and DNAs to obtain greatest stimulation of integrative recombination. If S20 is added before Int, or if Int is added before S20, less stimulation is observed. These results suggest S20 might be binding to Int and producing some kind of physical change that enhances its recombinase activity. Gel shift assays show that S20 binds to the DNA substrates in recombination assays. Thus, treatment of recombination assay mixtures containing large amounts of S20 with proteinase K is necessary to avoid trapping of DNA in wells during agarose gel electrophoresis. Titration of the amount of S20 versus number of recombinants obtained in both the integrative (Table 3) and excisive (Table 4) colony-forming recombination assay demonstrated rather sharp optima for amount of S20, particularly in the excisive reaction. The molar ratios of S20 to DNA nucleotides at the optimal amounts of S20 in these assays were 5 to 10 nucleotides per S20 molecule in the integrative reaction and 25 nucleotides per S20 molecule in the excisive reaction. It was speculated that the binding footprint for a protein of the size of S20 (about 10 kDa) functioning as a monomer is in the range of 5 to 10 nucleotides per molecule of protein. The optimum for the integrative reaction falls in this range, suggesting that for optimal stimulation of the integrative recombination sufficient S20 must be present to coat the DNA. Making the same assumptions, it would appear that in the excisive reaction, the presence of sufficient S20 to coat the DNA inhibits the reaction. In any case, binding of S20 to DNA is probably also exerting an effect on the efficiency of the recombination reaction, just as does the binding of other small nonspecific DNA binding proteins (Segall, A. M. et. al, EMBO J. 13:4536-4548 (1994)).

Part IV. Integrative Recombination Activity of Int and Int-His₆

Three purifications of native λ Int were completed following a modification (Materials and Methods) of the published purification procedure (Nash, H. A. Methods Enz. 100:210-216 (1983)), and a much larger number of purifications of cloned Int-His₆ by a simpler procedure (Materials and Methods). As a result of characterization of the integrative recombinase activity of these preparations using the gel assay (see Materials and Methods section Integrative Recombination Gel Assay), several general conclusions can be drawn about the activity of Int in the presence and absence of S20. First, preparations of Int or Int-His₆ that are nearly homogeneous and that are kept in a high salt (0.6 M KCl), low glycerol (10%) buffer during the final purification step (as recommended in the published purification procedure), and then are stored in that buffer in the presence or absence of BSA at −70° C., generally have reduced Int recombinase activity. But with all preparations tested, the activity can be increased by mixing Int with S20 before addition to an assay. It was found, however, that the activities of preparations of Int in the high salt buffer which appear lower can be increased to a certain extent by diluting the preparation in a low salt buffer (0.05 M KCl) before assay or more preferably by dialyzing the preparation into a buffer containing low salt (0.05 to 0.1 M KCl) and 50% (v/v) glycerol. Such preparations can then be stored at −20° C. or −70° C. Furthermore, regardless of the level of recombinase activity these preparations have by themselves before or after dialysis, addition of appropriate amounts of S20 stimulates that activity.

Conclusions

Taken together, these results demonstrate that at least two E. coli ribosomal proteins, S20 and L27, and possibly a third E. coli ribosomal protein, S15, stimulate λ Int-mediated recombination in vitro. In addition, purified preparations of λ Int that appear to be inactive in a X recombination system can be restored to activity by the addition of S20.

### Example 2

### Stimulation of Integrase Recombination by other E. coli Ribosomal Proteins

In addition to S20 and L27, other E. coli ribosomal proteins may stimulate the activity of recombination systems, particularly the λ Int system. In particular, E. coli ribosomal proteins that are basic and are about 14 kiloDaltons or less in size are used to stimulate the activity of prokaryotic recombination systems. Such ribosomal proteins that may be used are shown in Table 5:

TABLE 5
ADDITIONAL RIBOSOMAL PROTEINS FOR USE IN
STIMULATING RECOMBINATION ACTIVITY

| | | | Molecular |
|---|---|---|---|
| Ribosomal | No. of Basic Residues | No. of Total | Weight |
| Protein | (% of Total) | Residues | (Daltons) |
| S10 | 17 (16.5%) | 103 | 11,736 |
| S14 | 23 (23.7%) | 97 | 11,063 |
| S15 | 16 (18.4%) | 87 | 10,001 |
| S16 | 14 (17.1%) | 82 | 9,191 |
| S17 | 16 (19.3%) | 83 | 9,573 |
| S18 | 17 (23.0%) | 74 | 8,896 |
| S19 | 19 (20.9%) | 91 | 10,299 |
| S21 | 23 (32.9%) | 70 | 8,369 |
| L21 | 17 (16.5%) | 103 | 11,565 |
| L23 | 21 (21.2%) | 99 | 11,013 |
| L24 | 22 (21.4%) | 103 | 11,185 |
| L25 | 17 (18.1%) | 94 | 10,694 |
| L28 | 18 (23.4%) | 77 | 8,875 |
| L29 | 12 (19.0%) | 63 | 7,274 |
| L30 | 10 (17.2%) | 58 | 6,411 |
| L31 | 12 (19.4%) | 62 | 6,971 |
| L32 | 11 (19.6%) | 56 | 6,315 |
| L33 | 15 (27.8%) | 54 | 6,255 |
| L34 | 14 (30.4%) | 46 | 5,381 |

These ribosomal proteins are isolated from natural sources as generally described above for S20 and L27 and as discussed in Ann. Rev. Biochem 51:155 (1982), Ann. Rev. Biochem. 52:35 (1983), Ann. Rev. Biochem 53:75 (1984), and Ann. Rev. Biochem 66:679 (1997). Alternatively, the ribosomal proteins are prepared by recombinant DNA methodologies as generally outlined above for the production of S20 and Xis. Isolated ribosomal proteins are used to stimulate recombination activity, particularly that of Int, by including one or more of them in the compositions of the invention as described above for S20 and L27, and these compositions are used in integrative and excisive recombination assays, and in the recombinational cloning methods of the invention, as generally outlined in Example 1 for S20. In addition, ribosomal proteins corresponding to those described herein may be used in accordance with the invention. For example, ribosomal proteins from other prokaryotic sources, and from eukaryotic sources (e.g., yeast, fungi, animals (including mammals such as humans), plants, and the like) may be used in the methods and compositions of the invention.

### Example 3

### Escherichia coli Fis Protein Stimulates Integrative Recombination by Bacteriophage Lambda Int

Examples of the use of Fis to Stimulate Recombination

Addition of between 200 and 500 nM Fis to a standard BP CLONASE™ GATEWAY™ reaction will produce optimal stimulation of recombination product formation and number of output colonies. Similar levels of Fis will also stimulate reactions in which the topology of BP substrates are reversed; that is, using a linear P and supercoiled B substrate (library transfer). In both cases, the standard reaction conditions for the BP CLONASE™ reaction can be used. The same optimal range of Fis will also stimulate recombination reactions containing single P and B recombination sites under the same reaction conditions as reactions in the absence of Fis.

Summary of the Levels of Fis Stimulation of Recombination

A. Single Recombination Site Reactions

Optimal Fis stimulation is observed over a range of 200-500 nM Fis and 5 nM DNA. Fis stimulates all single-site integration reactions regardless of topology of substrates. The standard reaction using supercoiled attP and linear attB sites is stimulated up to 10-fold in the presence of lower levels of Int. The reverse topology reaction, using supercoiled attB and linear attP sites is stimulated up to 5-fold at various salt concentrations. The reaction between linear attP and linear attB sites is stimulated up to 3-fold by Fis.

B. Dual Recombination Site Reactions (GATEWAY™)

Optimal Fis stimulation is observed over a range of 200-500 nM Fis and 5 nM DNA. Fis stimulates the production of BP reaction product up to 3-fold depending on conditions. This stimulation appears to be due entirely to the stimulation of the resolution of the cointegrate, as cointegrate formation is unaffected. Standard GATEWAY™ reactions can be stimulated simply by adding Fis to the reaction under the same conditions as those normally used. In the reverse topology GATEWAY™ reaction (linear P, supercoiled B), Fis stimulates the production of product slightly, but significantly increases the amount of starting B substrate which is converted into cointegrate.

Results

Production of Fis

The E. coli fis gene was cloned into pLDE15 downstream of the lambda P_{L} promoter under control of the heat-inducible lambda cI857 repressor. This construct expressed Fis at high levels upon induction at 42° C. and a series of extracts were made to test purification protocols.

A final protocol was developed in which a liter of culture would produce 2-3 milligrams of purified (>90%) Fis. The procedure involved sonication to form a crude extract, followed by chromatography on Heparin sulfate, followed by ion-exchange chromatography on MonoS. The purified protein contains a few minor contaminants which could be further removed, possibly by either heating the extract before purification (as Fis is completely heat stable to boiling for up to 10 minutes), or by crystallization of Fis by complete dilution of salt. Both of these methods have been used in the literature. The final Fis sample was dialyzed into buffer containing 50% glycerol and 0.5M NaCl and was aliquoted into several tubes stored at either −20° C. or −80° C. The purified Fis was assayed for activity using a gel retardation assay similar to those published in the literature and found to have apparent Kd values obtained between 10-30 nM.

Effect of Fis on Excisive Recombination

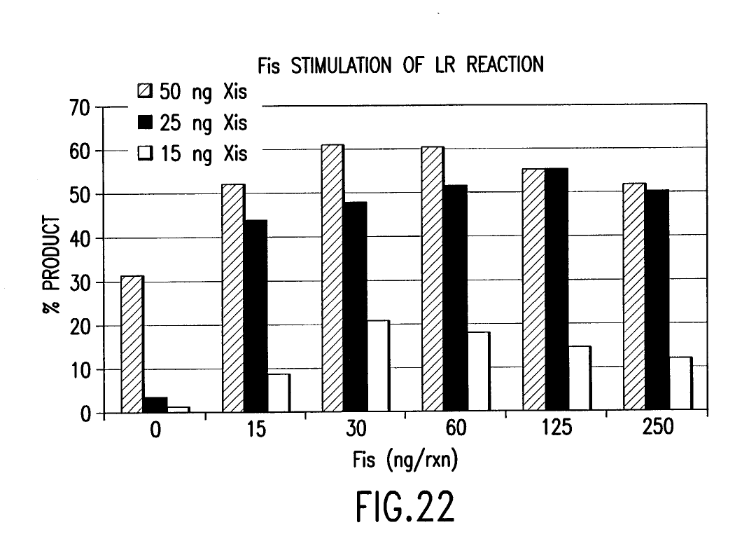

The effect of Fis on excision in vitro was measured using the double-site LR assay using supercoiled pEZ11104 (attL) and linearized pRCAT1 (attR). As shown in FIG. 22, increasing amounts of Fis protein showed a slight stimulation of the amount of recombinant product at high levels of Xis. However, as Xis levels were decreased, the stimulation by Fis was increased, such that at very limiting levels of Xis, maximal Fis stimulation reached 10-15 fold. Maximal stimulation by Fis seemed to occur between 30-125 ng Fis per 20 μl reaction. Because of the rapid conversion of cointegrate into product, it is difficult to analyze whether Fis affects both cointegrate formation and resolution; however, it is likely that stimulation is observed at both steps, and the level of stimulation appears to be similar.

Effect of Fis on Integrative Cointegrate Resolution.

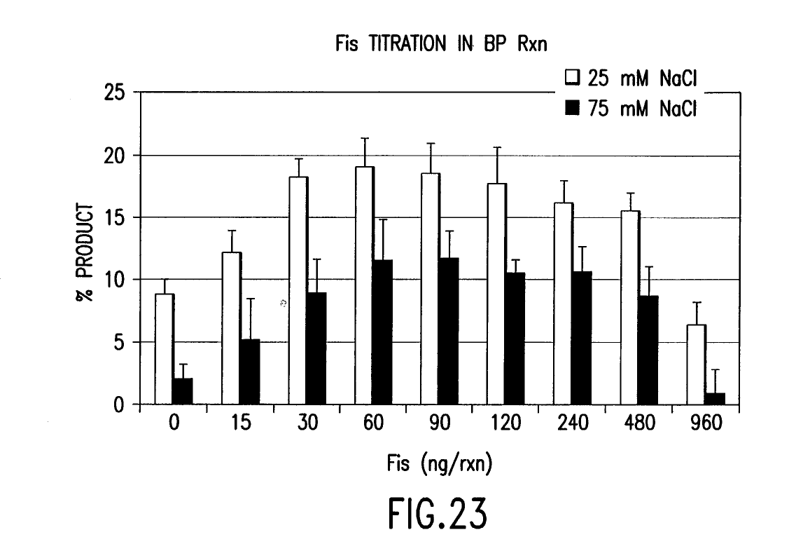

FIG. 23 shows the effect of Fis addition to a double-site BP assay using supercoiled pDONR201 (attP) and linearized pBGFP1 (attB). The percentage of recombination products is increased 2-4 fold in the presence of optimal levels of Fis (again, 30-120 ng/reaction). Also, stimulation by Fis is greater at higher salt, which is a condition that normally disfavors cointegrate resolution. There is no observable effect on cointegrate formation in the presence of Fis at any salt concentration (data not shown).

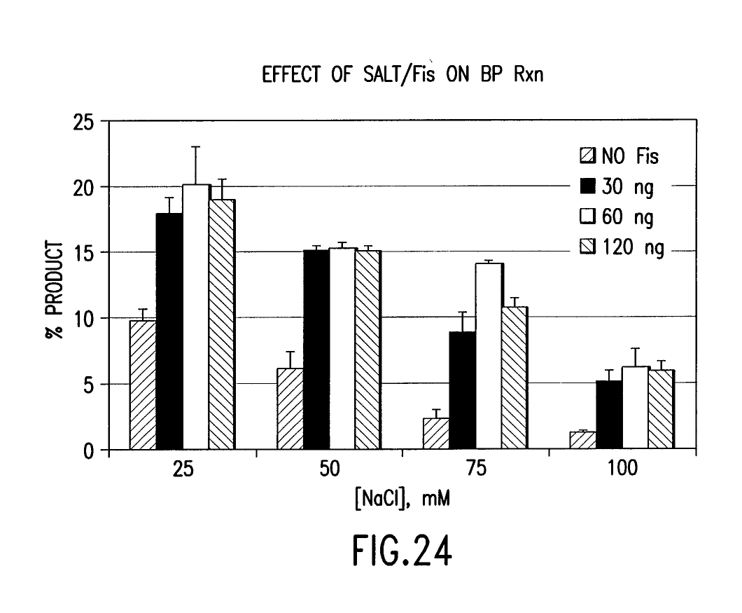

FIG. 24 analyzes the effect of salt concentration in more detail. Once again, the stimulation by Fis is seen at all salt concentrations, but because the control in the absence of Fis is so dramatically affected by salt concentration, the stimulation by Fis at higher salt is much stronger. At 25 mM NaCl, Fis stimulates nearly 2-fold, while at 75 and 100 mM NaCl, Fis stimulation is greater than 7-fold. In no case, however, is the amount of recombinant product at higher salt higher than the optimal Fis-stimulated recombination at 25 mM NaCl.

Effect of Fis on Integrative Recombination

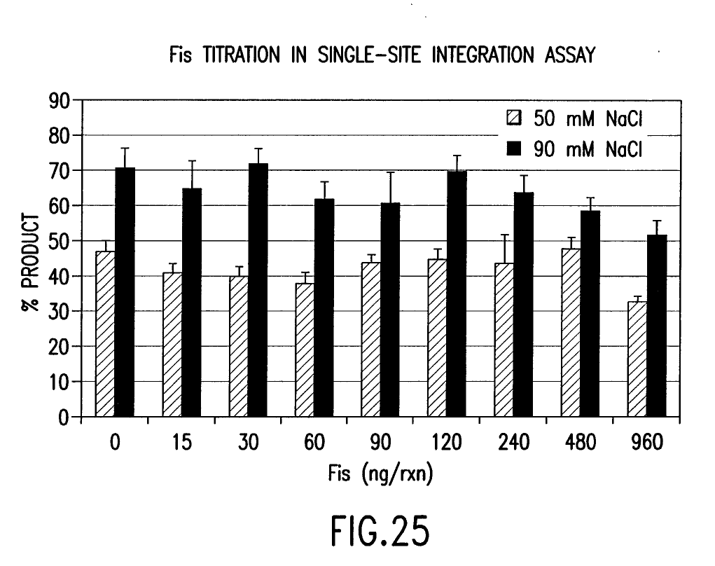

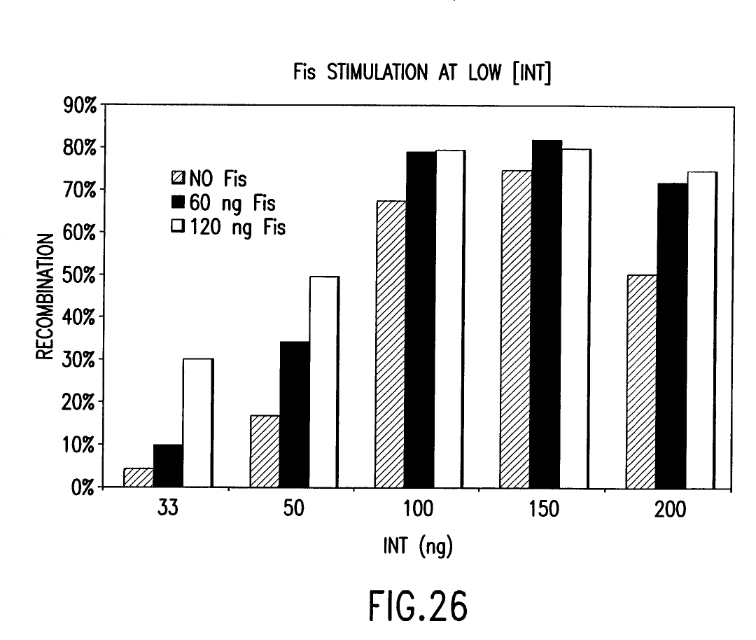

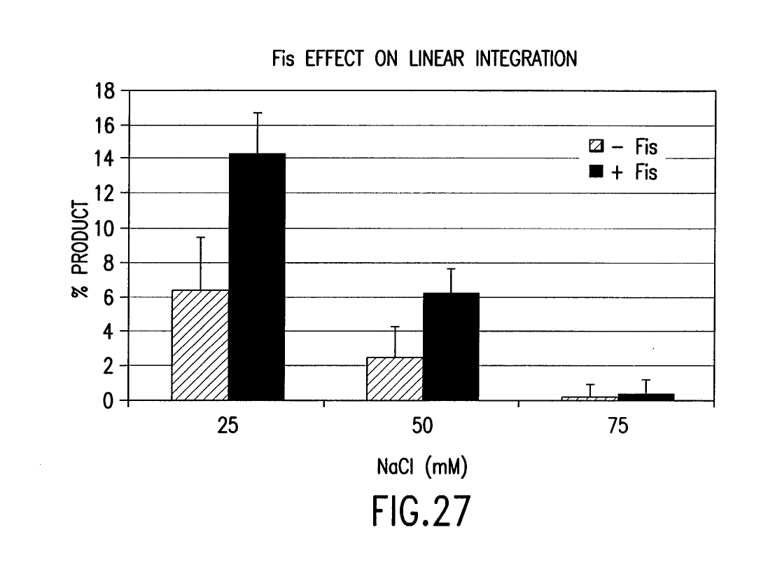

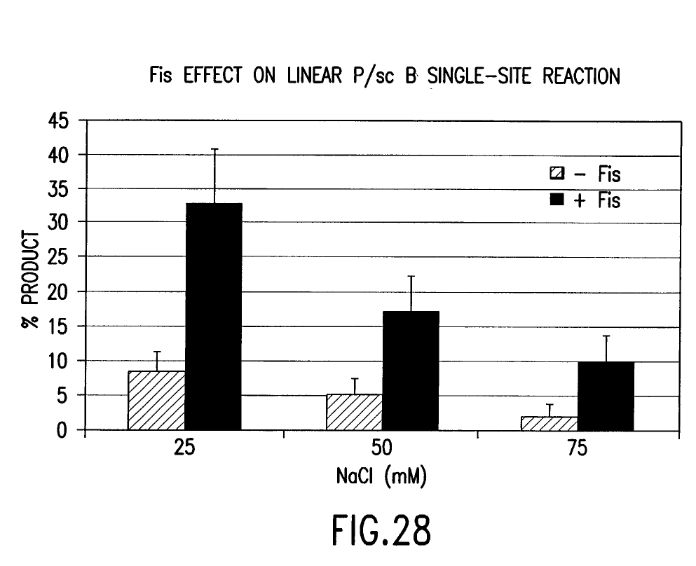

FIG. 25 shows that Fis has no effect on single-site PxB recombination under standard conditions where attP (pATTP2) is supercoiled, and attB (pATTB2) is linear, at either low or high salt. However, if the levels of Int are reduced to suboptimal concentrations (FIG. 26), Fis is now capable of stimulating this reaction up to 10-fold. In addition, as FIG. 27 shows, when both substrates are linearized, Fis has a dramatic effect on recombination levels. With linearized pATTP2 and linearized pATTB2, Fis stimulates recombination 2-3 fold at varying salt concentrations, much like the results seen for cointegrate resolution reactions. The most significant effect of Fis seems to be on the reaction between supercoiled pATTB2 and linear pATTP2. This reaction is extremely poor under normal conditions, with barely detectable amounts of product observed even at low salt conditions. However, in the presence of Fis, as shown in FIG. 28, recombination is strongly stimulated.

Discussion

The results of this study identified the likely source of the stimulation observed in vivo during integration. A 2-3 fold effect is clearly observed in vitro when attP substrates are not supercoiled. It has long been known that supercoiling energy appears to be essential for proper establishment of the protein-DNA structure known as the intasome, which is required to form prior to the onset of recombination. This argument has been used to explain the much lower recombination efficiency observed with non-supercoiled attP substrates in vitro. However, it has been widely shown that DNA in the cell is not supercoiled to the high levels of superhelicity seen in isolated plasmid DNA.

Johnson first proposed the notion that Fis may be used in the cell to enhance integration under conditions where such high superhelicity is not present (Ball, C. A. and Johnson, R. C. (1991) J. Bacteriol. 173:4032-8). Given the fact that many nucleoid associated proteins appear to be involved in DNA compaction of the nucleoid, it is possible that the ability of Fis to bind and bend DNA may well mimic the compaction of DNA by supercoiling, and such an event may allow proper intasome formation even in the absence of high superhelicity. This may also be the explanation for the stimulation by Fis observed at suboptimal Int concentrations. In the cell, where Int levels are likely to be much lower than the artificially high concentrations used in laboratory in vitro recombination reactions, Fis may be necessary even for a “standard” recombination reaction to proceed.

The ability of F site mutants to promote stronger Fis stimulation of integration is further evidence for the role proposed above. Tighter Fis binding would likely lead to more efficient compaction of the DNA, and an increase in integration stimulation. It remains to be seen whether these effects are manifested at the kinetic level-that is, does the addition of Fis directly speed up intasome formation? Initial studies point towards an increase in the initial rate of the linear attP/supercoiled attB reaction in the presence of Fis, suggesting that indeed Fis may be kinetically acting at the level of intasome formation.

It is not entirely clear why Fis seems to have a greater stimulation of linear P/supercoiled B reactions as compared to reactions in which both substrates are linear. It is believed that integrative intasome formation occurs solely on attP, with capture of attB being a final step in the synapsis process. In this case, it is unclear how the supercoiling state of attB could affect the outcome of intasome formation. Instead, it is possible that Fis interaction with attB somehow makes the attB sites more accessible to the intasome, or aids a downstream post-synapsis step such as isomerization after the first strand cleavage.

Experimental Methods

Oligonucleotides

Oligonucleotides were obtained from Invitrogen Corp., Life Technologies Division (Rockville, Md.).

| | | |
|---|---|---|
| DE09: | 5′-GGGGGCTGCAGGCAAGAAGACAAAAATCACCTTGCGC | (SEQ ID NO:17) |
| DE10: | 5′-GGGGGCCCGGGCAGAGGCAGGGAGTGGGACAAAATTG | (SEQ ID NO:18) |
| DE46 (Fis start): | 5′-GGAGGGAATTCAGGAGGTATAAATTAATGTTCGAACAACGCGTAAATTCTG | (SEQ ID NO:198) |
| DE49 (Fis stop): | 5′-GGAGGGGATCCTTATTAGTTCATGCCGTA | (SEQ ID NO:20) |
| DE162: | 5′-GGAAGGAGATCTTGCTCAAAATTTGAGCTACATAATACTGTAAAACAC | (SEQ ID NO:21) |

Recombination Assay Plasmids

pATTP2 was constructed by cloning the lambda attP site into pUC19. pATTB2 was constructed by cloning the E. coli attB site into pUC19. pDONR201 (Invitrogen Corp., Life Technologies Division (Rockville, Md.), Catalog No. 11798-014) contains attP1 and attP2 sites flanking a ccdB gene. pEZ11104 contains attL1 and attL2 sites flanking a CAT gene. pBGFP2 is pUC19 into which a PCR fragment containing the attB 1 and attB2 sites flanking the GFP gene has been inserted. pRCAT1 is pUC19 into which a fragment of pEZC8402 containing the attR1 and attR2 sites and the CAT/ccdB cassette has been inserted.

Cloning of E. coli Fis.

The fis gene was PCR amplified from E. coli DH10B chromosomal DNA using Platinum Taq Hi Fidelity, and primers (DE46 and DE49) corresponding to the 5′ and 3′ ends of the gene. The 5′ primer was constructed to provide a strong Shine-Delgarno initiation sequence prior to the start of the fis gene. The PCR product was digested and cloned into pRAD19, a high copy-number expression vector carrying the lambda PL promoter under the control of the heat-inducible lambda CI⁸⁵⁷ gene. A positive clone (pLDE15) was sequence verified to ensure that no mutations were present, and was introduced into E. coli BL21 for expression.

Induction of E. coli Fis Protein

Cells containing pLDE15 were grown overnight at 30° C. in 2 milliliters of LB with 100 μg/ml ampicillin, diluted into 2 milliliters of fresh media, and grown to an OD₆₀₀ of 0.7. The culture was split into 2 tubes, with one remaining at 30° C., with the other induced at 42° C. for 2 hours. After 2 hours, the cultures were spun down, resuspended in loading buffer, and analyzed by SDS-PAGE. The induced cells already had a partially lysed appearance, suggesting that dramatic overexpression of Fis may be lethal to E. coli under these conditions. Induced samples showed a very clearly overexpressed protein band at a molecular weight of around 12 kDa.

Purification of E. coli Fis Protein

A 5 ml overnight culture of pLDE15 was diluted into 1 liter LB+Amp in a Fernbach flask, and was grown at 30° C. to an OD₆₀₀ of 0.7, induced at 42° C. for 2 hours, and spun down. 7.5 g of wet cells were obtained, and were frozen at −80° C. Cells were thawed and resuspended in 15 milliliters of buffer containing 50 mM Tris-HCl, pH 8.0, 5 mM EDTA, 10% glycerol, 1 M NaCl, and 1 mM DTT. The cell solution was sonicated 4 times for 45 seconds with a ½ inch tip, and debris was removed by centrifugation at 30,000× g for 40 minutes. Extracts were stored at −80° C. 15 milliliters of extract was diluted with 35 milliliters buffer A (20 mM Tris-HCl, pH 8.0, 1 mM EDTA, 10% glycerol, 1 mM DTT) and applied to a Pharmacia Hitrap Heparin column (2×1 ml columns in series) at a flow rate of 0.25 ml/min. The column was washed with 400 mM NaCl in buffer A for 10 CV, and eluted with a 15 CV gradient from 400 mM to 800 mM NaCl in buffer A. A broad peak of Fis was detected by SDS-PAGE and fractions containing Fis were pooled, and dialyzed against buffer A with 200 mM NaCl. This sample was applied to a 1 ml Pharmacia Hitrap MonoS column equilibrated in the same buffer. The column was washed with 15 CV of 200 mM NaCl in buffer A, and eluted with a 20 CV gradient of 200 mM to 1M NaCl in buffer A. Two peaks were observed from the column, with the second sharp peak representing most of the Fis protein. The cleanest fractions were pooled to give a sample containing >90% Fis by Coommassie staining. Purified Fis was obtained at 1 mg/ml concentration after dialysis into Fis storage buffer containing 20 mM Tris-HCl, pH 8.0, 1 mM EDTA, 50% glycerol, 1 mM DTT, 0.5 M NaCl. Fis was stored at −80° C. or −20° C.

Fis Activity Assay

A gel retardation assay was developed to test for Fis activity. A PCR product consisting of the lambda attP sequence was amplified using primers DE9 and DE10. The 400 base pair product was cut with AvaI and labeled at the ends with ³²P-dCTP using the Klenow fragment of E. coli DNA polymerase I. Reactions were carried out with final conditions of 20 mM Tris-HCl, pH 8.0, 5% glycerol, 25 mM NaCl, 200 μg/ml salmon testis DNA, 1.17 ng (10,000 cpm/fmol) PCR product in a 20 μl reaction. Protein was added, and binding was carried out for 10 minutes at room temperature, and samples were loaded on a Novex 6% gel retardation gel running in 0.5× TBE buffer for 60 minutes at 100 V. Gels were dried and visualized on the Phosphorimager after 2-3 hour exposure. Multiple shifts were observed in assays without competitor DNA. In the presence of competitor, however, a single discrete shift was observed, and allowed the calculation of an apparent Kd value. These PCR products were somewhat impure, containing breakdown products, and the values obtained were therefore slightly error prone; however, the apparent Kd appeared to be between 10-30 nM, which agrees well with published values using the lamdba F site. This suggests that this kind of gel retardation assay would serve as an effective check of the activity of purified Fis protein.

Radioactive Assay Substrates

Linear substrates for recombination assays were labeled by Klenow fill-in reactions. Linearized substrates (1 μg) were incubated with 0.5 units of Klenow polymerase, 1 mM dATP, 1 mM dGTP, 1 mM dTTP, and 30 μCi of ³²P-dCTP for 14 minutes, 1 mM dCTP was added, incubated for 1 minute, and the labeled DNA was purified using Concert PCR purification columns, and eluted in 50 μl TE.

Recombination Assays.

Single-site recombination reactions (20 1) consisted of 25 mM Tris-HCl, pH 8.0, 1 mM EDTA, 6 mM spermidine, 15% glycerol, and 75 mM NaCl (unless indicated otherwise), 100 fmoles of each substrate, and approximately 30,000 cpm of ³²P-labelled linear substrate. Standard integration reactions contained 80 ng IHF and 150 ng Int. Excision reactions contained 35 ng IHF, 50 ng Xis, and 150 ng Int. Reactions were incubated for 45 minutes at 25° C., and stopped by the addition of 50 μg/ml Proteinase K, heated for 15 minutes at 65° C., and electrophoresed on a 0.7% agarose gel. Gels were dried down and visualized on a Molecular Dynamics phosphorimager. Recombination levels were determined by quantitation of substrate and product bands using ImageQuant. GATEWAY™ (2-site) reactions were performed similarly, except that standard BP reactions contained 4 mM spermidine and 25 mM NaCl, and standard LR reactions contained 7.5 mM spermidine and 75 mM NaCl.

### Example 4

### Use of Fis in BP CLONASE™ Reactions

BP recombination reactions were performed for 60-120 minutes at room temp in 20 μl reaction mixtures containing 50 fmol supercoiled pDONR201, 75 mM NaCl, 7.5 mM spermidine, 2 μl BP storage buffer (5 mM EDTA, 1 mg/ml BSA, 22 mM NaCl, 5 mM spermidine, 25 mM Tris-HCl, pH 7.5) and 2 μl BP CLONASE™ (40 ng/μl Int, 20 ng/μl IHF, pH 7.5). The optimal Fis concentration for enhancing the efficiency of BP CLONASE™ catalyzed recombination reaction was found to be about 150 nM.

Further, the above reaction conditions generate a colony output that is similar to the standard reaction (i.e., 300 ng pDONR DNA, 100 ng attB DNA, 4 μl BP CLONASE™, 4 μl BP buffer for a 20 μl reaction), but requires half the amount of enzyme and vector DNA.

In a standard BP recombination reaction, addition of Fis results in a 3-fold increase in colony output as compared to from a standard BP reaction.

Fis is known to exert its effect by stimulating the rate of the second recombination reaction (cointegrate resolution) which is a linear by linear recombination reaction.

While not wishing to be bound by theory, the overall efficiency of BP recombination reactions involving linear and supercoiled nucleic acid molecules is as follows:

Supercoiled P×Linear B>Linear P×Supercoiled B>Linear P×Linear B>Supercoiled P×Supercoiled B

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application is specifically and individually indicated to be incorporated by reference.

What is claimed is:

1. A composition comprising at least one recombination protein and at least one Fis protein or Fis protein fragment, wherein the recombination protein is present in an amount effective for recombinational cloning of at least one nucleic acid molecule and the Fis protein or Fis protein fragment is present in an amount effective for enhancing the efficiency of the recombinational cloning.

2. The composition of claim 1, further comprising at least one ribosomal protein or ribosomal protein fragment, wherein the ribosomal protein or ribosomal protein fragment is present in an amount effective for enhancing the efficiency of the recombinational cloning.

3. The composition of claim 2, wherein the ribosomal protein is a prokaryotic ribosomal protein.

4. The composition of claim 2, wherein the ribosomal protein is an Escherichia coli ribosomal protein.

5. The composition of claim 4, wherein the E. coli ribosomal protein is a protein selected from the group of E. coli ribosomal proteins consisting of S10, S14, S15, S16, S17, S18, S19, S20, S21, L14, L21, L23, L24, L25, L27, L28, L29, L30, L3 1, L32, L33 and L34.

6. The composition of claim 5, wherein the E. coli ribosomal protein is S20.

7. The composition of claim 5, wherein the E. coli ribosomal protein is L27.

8. The composition of claim 5, wherein the E. coli ribosomal protein is S15.

9. The composition of claim 2, wherein the ribosomal protein is a basic ribosomal protein.

10. The composition of claim 2, wherein the ribosomal protein or ribosomal protein fragment has a molecular weight of less than about 14 kiloDaltons.

11. The composition of claim 1, wherein the Fis protein is a Fis protein of an organism selected from the group consisting of:
(a) Escherichia coli;
(b) Salmonella typhimurium;
(c) Klebsiella pneumoniae;
(d) Vibrio cholera;
(e) Haemophilus influenza; and
(f) Pseudomonas aeruginosa.

12. The composition of claim 1, wherein the Fis protein comprises an amino acid sequence at least 90% identical to an amino acid sequence selected from the group consisting of:
(a) the amino acid sequence of SEQ ID NO:1;
(b) the amino acid sequence of SEQ ID NO:2;
(c) the amino acid sequence of SEQ ID NO:3;
(d) the amino acid sequence of SEQ ID NO:4;
(e) the amino acid sequence of SEQ ID NO:5; and
(f) the amino acid sequence of SEQ ID NO:6.

13. The composition of claim 12, wherein the Fis protein comprises an amino acid sequence selected from the group consisting of:
(a) the amino acid sequence of SEQ ID NO:1;
(b) the amino acid sequence of SEQ ID NO:2;
(c) the amino acid sequence of SEQ ID NO:3;
(d) the amino acid sequence of SEQ ID NO:4;
(e) the amino acid sequence of SEQ ID NO:5; and
(f) the amino acid sequence of SEQ ID NO:6.

14. The composition of claim 1, wherein the Fis protein fragment comprises at least 15 amino acid residues of an amino acid sequence selected from the group consisting of:
(a) the amino acid sequence of SEQ ID NO:1;
(b) the amino acid sequence of SEQ ID NO:2;
(c) the amino acid sequence of SEQ ID NO:3;
(d) the amino acid sequence of SEQ ID NO:4;
(e) the amino acid sequence of SEQ ID NO:5; and
(f) the amino acid sequence of SEQ ID NO:6.

15. The composition of claim 1, wherein the recombination protein is a prokaryotic recombination protein.

16. T he composition of claim 1, wherein the recombination protein is selected from the group consisting of Int, Cre, FLP, Xis, IHF and HU, and combinations thereof.

17. The composition of claim 16, wherein the recombination protein is Int.

18. The composition of claim 1, further comprising at least one nucleic acid molecule.

19. The composition of claim 18, wherein the nucleic acid molecule is a linear nucleic acid molecule.

20. The composition of claim 18, wherein the nucleic acid molecule is a closed, circular nucleic acid molecule.

21. The composition of claim 18, wherein the nucleic acid molecule is a vector.

22. The composition of claim 18, wherein the nucleic acid molecule comprises a molecule selected from the group consisting of:
(a) an Insert Donor molecule;
(b) a Vector Donor molecule;
(c) a Cointegrate molecule;
(d) a Product molecule; and
(e) a Byproduct molecule.

23. A method for recombinational cloning of at least one first nucleic acid molecule, the method comprising:
(a) forming a mixture by mixing the first nucleic acid molecule with at least one second nucleic acid molecule and with the composition of claim 1; and
(b) incubating the mixture formed in (a) under conditions sufficient to recombine the first nucleic acid molecule with the second nucleic acid molecule,
wherein the first nucleic acid molecule and the second nucleic acid molecule each comprise at least one recombination site.

24. A method for recombinational cloning of at least one first nucleic acid molecule, the method comprising:
(a) forming a mixture by mixing the first nucleic acid molecule with at least one second nucleic acid molecule and with the composition of claim 2; and
(b) incubating the mixture formed in (a) under conditions sufficient to recombine the first nucleic acid molecule with the second nucleic acid molecule,
wherein the first nucleic acid molecule and the second nucleic acid molecule each comprise at least one recombination site.

25. The method of claim 23, wherein the first nucleic acid molecule is genomic DNA.

26. The method of claim 23, wherein the first nucleic acid molecule is cDNA.

27. The method of claim 23, wherein the first nucleic acid molecule is produced by chemical synthesis.

28. The method of claim 23, wherein the first nucleic acid molecule is produced by amplification.

29. The method of claim 23, wherein the second nucleic acid molecule is a vector.

30. The method of claim 29, wherein the vector is capable of replicating in prokaryotic cells, eukaryotic cells, or both prokaryotic and eukaryotic cells.

31. The method of claim 30, wherein the vector is capable of replicating in yeast cells, plant cells, fish cells, eukaryotic cells, mammalian cells, and/or insect cells.

32. The method of claim 30, wherein the vector is capable of replicating in bacteria of the genera Escherichia, Salmonella, Bacillus, Streptomyces, and/or Pseudomonas.

33. The method of claim 32, wherein the vector is capable of replicating in E. coli.

34. A method for enhancing recombinational cloning reactions, the method comprising contacting at least two nucleic acid molecules with at least one Fis protein or Fis protein fragment and at least one recombination protein, wherein the nucleic acid molecules comprise at least one recombination site.

35. The method of claim 34, further comprising contacting the nucleic acid molecules with at least one ribosomal protein or ribosomal protein fragment.

36. The method of claim 35, wherein the ribosomal protein is a prokaryotic ribosomal protein.

37. The method of claim 36, wherein the ribosomal protein is an Escherichia coli ribosomal protein.

38. The method of claim 37, wherein the E. coli ribosomal protein is a protein selected from the group of E. coli ribosomal proteins consisting of S1, S14, S15, S16, S17, S18, S19, S20, S21, L14, L21, L23, L24, L25, L27, L28, L29, L30, L31, L32, L33 and L34.

39. The method of claim 38, wherein the E. coli ribosomal protein is S20.

40. The method of claim 38, wherein the E. coli ribosomal protein is L27.

41. The method of claim 38, wherein the E. coli ribosomal protein is S15.

42. The method of claim 35, wherein the ribosomal protein is a basic ribosomal protein.

43. The method of claim 35, wherein the ribosomal protein or ribosomal protein fragment has a molecular weight of less than about 14 kiloDaltons.

44. The method of claim 34, wherein the Fis protein is a Fis protein of an organism selected from the group consisting of:
(a) Escherichia coli;
(b) Salmonella typhimurium;
(c) Klebsiella pneumoniae;
(d) Vibrio cholera;
(e) Haemophilus influenza; and
(f) Pseudomonas aeruginosa.

45. The method of claim 34, wherein the Fis protein comprises an amino acid sequence at least 90% identical to an amino acid sequence selected from the group consisting of:
(a) the amino acid sequence of SEQ ID NO:1;
(b) the amino acid sequence of SEQ ID NO:2;
(c) the amino acid sequence of SEQ ID NO:3;
(d) the amino acid sequence of SEQ ID NO:4;
(e) the amino acid sequence of SEQ ID NO:5; and
(f) the amino acid sequence of SEQ ID NO:6.

46. The method of claim 45, wherein the Fis protein comprises an amino acid sequence selected from the group consisting of:
(a) the amino acid sequence of SEQ ID NO:1;
(b) the amino acid sequence of SEQ ID NO:2;
(c) the amino acid sequence of SEQ ID NO:3;
(d) the amino acid sequence of SEQ ID NO:4;
(e) the amino acid sequence of SEQ ID NO:5; and
(f) the amino acid sequence of SEQ ID NO:6.

47. The method of claim 34, wherein the Fis protein fragment comprises at least 15 amino acid residues of an amino acid sequence selected from the group consisting of:
(a) the amino acid sequence of SEQ ID NO:1;
(b) the amino acid sequence of SEQ ID NO:2;
(c) the amino acid sequence of SEQ ID NO:3;
(d) the amino acid sequence of SEQ ID NO:4;
(e) the amino acid sequence of SEQ ID NO:5; and
(f) the amino acid sequence of SEQ ID NO:6.

48. The method of claim 34, wherein the recombination protein is a prokaryotic recombination protein.

49. The method of claim 34, wherein the recombination protein is selected from the group consisting of Int, Cre, FLP, Xis, IHF and HU, and combinations thereof.

50. The method of claim 49, wherein the recombination protein is Int.

51. A DNA molecule produced by the method of claim 34.

52. The DNA molecule of claim 51, wherein the DNA molecule is an isolated DNA molecule.

53. A host cell comprising the DNA molecule of claim 51.

54. A method for cloning at least one nucleic acid molecule comprising a nucleic acid segment flanked by at least two recombination sites, wherein the recombination sites do not substantially recombine with each other, the method comprising:
(a) forming a combination by combining in vitro or in vivo:
(i) at least one Insert Donor molecule comprising the nucleic acid molecule;
(ii) at least one first Vector Donor molecule comprising at least two recombination sites, wherein the recombination sites do not substantially recombine with each other;
(iii) an effective amount of at least one recombination protein; and
(iv) an effective amount of at least one Fis protein or Fis protein fragment; and
(b) incubating the combination under conditions sufficient to transfer the nucleic acid molecule into the first Vector Donor molecule, thereby producing at least one first Product molecule.

55. The method of claim 54, further comprising:
(c) forming a combination by combining in vitro or in vivo:
(i) the first Product molecule comprising the nucleic acid molecule;
(ii) at least one second Vector Donor molecule comprising two or more recombination sites, wherein the recombination sites do not substantially recombine with each other;
(iii) an effective amount of at least one recombination protein; and
(iv) an effective amount of at least one Fis protein or Fis protein fragment; and
(d) incubating the combination under conditions sufficient to transfer the nucleic acid molecule into the second Vector Donor molecule, thereby producing at least one second Product molecule.

56. The method of claim 54, wherein the combination formed in step (a) further comprises at least one ribosomal protein or ribosomal protein fragment.

57. The method of claim 56, wherein the ribosomal protein is a prokaryotic ribosomal protein.

58. The method of claim 56, wherein the ribosomal protein is an Escherichia coli ribosomal protein.

59. The method of claim 58, wherein the E. coli ribosomal protein is a protein selected from the group of E. coli ribosomal proteins consisting of S1, S14, S15, S16, S17, S18, S19, S20, S21, L14, L21, L23, L24, L25, L27, L28, L29, L30, L31, L32, L33 and L34.

60. The method of claim 59, wherein the E. coli ribosomal protein is S20.

61. The method of claim 59, wherein the E. coli ribosomal protein isL27.

62. The method of claim 59, wherein the E. coli ribosomal protein is S15.

63. The method of claim 56, wherein the ribosomal protein is a basic ribosomal protein.

64. The method of claim 56, wherein the ribosomal protein or ribosomal protein fragment has a molecular weight of less than about 14 kiloDaltons.

65. The method of claim 54, wherein the Fis protein is a Fis protein of an organism selected from the group consisting of:
(a) Escherichia coli;
(b) Salmonella typhimurium;
(c) Klebsiella pneumoniae;
(d) Vibrio cholera;
(e) Haemophilus influenza; and
(f) Pseudomonas aeruginosa.

66. The method of claim 54, wherein the Fis protein comprises an amino acid sequence at least 90% identical to an amino acid sequence selected from the group consisting of:
(a) the amino acid sequence of SEQ ID NO:1;
(b) the amino acid sequence of SEQ ID NO:2;
(c) the amino acid sequence of SEQ ID NO:3;
(d) the amino acid sequence of SEQ ID NO:4;
(e) the amino acid sequence of SEQ ID NO:5; and
(f) the amino acid sequence of SEQ ID NO:6.

67. The composition of claim 66, wherein the Fis protein comprises an amino acid sequence selected from the group consisting of:
(a) the amino acid sequence of SEQ ID NO:1;
(b) the amino acid sequence of SEQ ID NO:2;
(c) the amino acid sequence of SEQ ID NO:3;
(d) the amino acid sequence of SEQ ID NO:4;
(e) the amino acid sequence of SEQ ID NO:5; and
(f) the amino acid sequence of SEQ ID NO:6.

68. The composition of claim 54, wherein the Fis protein fragment comprises at least 15 amino acid residues of an amino acid sequence selected from the group consisting of:
(a) the amino acid sequence of SEQ ID NO:1;
(b) the amino acid sequence of SEQ ID NO:2;
(c) the amino acid sequence of SEQ ID NO:3;
(d) the amino acid sequence of SEQ ID NO:4;
(e) the amino acid sequence of SEQ ID NO:5; and
(f) the amino acid sequence of SEQ ID NO:6.

69. A kit for use in recombinational cloning of a nucleic acid molecule, the kit comprising at least one Fis protein or Fis protein fragment.

70. The kit of claim 69 which further comprises at least one component selected from the group consisting of:
(a) at least one nucleic acid molecule;
(b) at least one recombination protein or compositions comprising at least one recombination protein;
(c) at least one enzyme having ligase activity;
(d) at least one enzyme having polymerase activity;
(e) at least one enzyme having reverse transcriptase activity;
(f) at least one enzyme having restriction endonuclease activity;
(g) at least one ribosomal protein or ribosomal protein fragment;
(h) at least one primer;
(i) at least one buffer;
(j) at least one transfection reagent;
(k) at least one host cell;
(l) at least one recombination protein; and
(m) instructions for using the kit components.

71. The kit of claim 70, wherein the at least one recombination protein or composition comprising at least one recombination protein is capable of catalyzing recombination between att sites.

72. The kit of claim 71, wherein the composition comprising at least one recombination protein is capable of catalyzing a BP reaction, an LR reaction, or both BP and LR reactions.

73. The kit of claim 70, wherein the ribosomal protein is a prokaryotic ribosomal protein.

74. The kit of claim 73, wherein the ribosomal protein is an Escherichia coli ribosomal protein.

75. The kit of claim 74, wherein the E. coli ribosomal protein is a protein selected from the group of E. coli ribosomal proteins consisting of SO, S14, S15, S16, S17, S18, S19, S20, S21, L14, L21, L23, L24, L25, L27, L28, L29, L30, L31, L32, L33 and L34.

76. The kit of claim 75, wherein the E. coli ribosomal protein is S20.

77. The kit of claim 78, wherein the E. coli ribosomal protein is L27.

78. The kit of claim 78, wherein the E. coli ribosomal protein is S15.

79. The kit of claim 73, wherein the ribosomal protein is a basic ribosomal protein.

80. The kit of claim 73, wherein the ribosomal protein or ribosomal protein fragment has a molecular weight of less than about 14 kiloDaltons.
